(19) United States
(12) Patent Application Publication
(10) Pub. No.: US 2026/0317393 A1
(43) Pub. Date: Oct. 15, 2026

(54) METHOD AND SYSTEM FOR PREDICTING COUPLING PROBABILITIES OF G-PROTEIN COUPLED RECEPTORS WITH G-PROTEINS

(71) Applicant: CELL NETWORKS GMBH, Heidelberg (DE)

(72) Inventors: Robert Bruce RUSSELL, Heidelberg (DE); Asuka INOUE, Miyagi (JP)

(21) Appl. No.: 19/334,630

(22) Filed: Sep. 19, 2025

Related U.S. Application Data
(62) Division of application No. 17/615,000, filed on May 29, 2020, now Pat. No. 12,612,442.

(30) Foreign Application Priority Data
May 29, 2019 (EP) 19177387.8

(51) Int. Cl. C07K 14/705; C12N 9/12; C12N 9/14

(52) U.S. Cl. CPC C07K 14/705; C12N 9/12; C12N 9/14; C12Y 207/11013; C12Y 306/05002

(57) ABSTRACT

Disclosed is a Designer Receptor Exclusively Activated by Designer Drugs (DREADD), and an amino acid sequence for determining coupling or no coupling between G-protein and G-protein coupled receptor (GPCR) mediated by a GPCR ligand in a cell based assay.

### CROSS REFERENCE TO RELATED APPLICATION

The present invention is a divisional of U.S. patent application Ser. No. 17/615,000, filed Nov. 29, 2021, which in turn is a 371 US National Phase Patent Application of PCT/EP2020/064937, Filed May 29, 2020, which claims benefit to European Patent Application Serial no. 19177387.8, filed May 29, 2020, the contents of which are incorporated herein by reference.

### REFERENCE TO AN ELECTRONIC SEQUENCE LISTING

The contents of the electronic sequence listing (PRINZ C4692 DIV.xml; Size: 172,000 bytes; and Date of Creation: Apr. 9, 2026) is herein incorporated by reference in its entirety.

### FIELD OF INVENTION

The present invention relates to a Designer Receptor Exclusively Activated by Designer Drugs (DREADD), and an amino acid sequence for determining coupling or no coupling between G-protein and G-protein coupled receptor (GPCR) mediated by a GPCR ligand in a cell based assay.

### BACKGROUND

G-protein coupled receptors (GPCRs), one of the largest protein superfamilies, are key mediators linking extracellular ligands to downstream signals and are the most common targets for pharmaceutical drug development (Hauser et al., 2017; Hauser et al., 2018). Ligand binding induces conformational changes in GPCRs that then lead to intracellular binding by particular heterotrimeric G-protein complexes, each consisting of Gα, Gβ and Gγ subunits, where distinct Gα subunits specify both GPCR interactions and the transduction of particular downstream signaling events (Wettschureck and Offermanns, 2005). The human genome encodes 16 Gα genes that are grouped into four subfamilies Gαₛ, Gα_{i/o}, Gα_{q/11} and Gα_{12/13}, that capture broad properties of downstream signaling (e.g., adenylate cyclase activation by Gαₛ) (Wettschureck and Offermanns, 2005). In general, each of the hundreds of mammalian GPCRs couple with more than one G-protein giving each a distinct coupling profile (Harding et al., 2018), or signature, which evokes a unique cellular response. Determining these GPCR profiles is critical to understanding their biology and pharmacology.

Pharmaceutical interest in GPCRs has prompted many efforts during the last decades to determine both their ligands and signaling (Hauser et al., 2018). Among approximately 360 non-sensory GPCR genes encoded in the human genome, one-third are still labelled as orphans to reflect the fact that either ligands and/or signaling are unknown (Harding et al., 2018). Previous efforts to uncover signaling profiles have been laborious and not standardized, yet tended to identify only the subfamily or signaling outcome (e.g. Ca²⁺, CAMP, inositol phosphate, Rho activation), rather than the specific Ga subunit binding event (Thomsen et al., 2005). Although this has led a collection of data on GPCR ligands and signaling exemplified in the IUPHAR/BPS Guide to Pharmacology (GtoPdb) (Harding et al., 2018), these databases have issues with mixed quality of G-protein coupling data as well as lack of “negative” coupling information. Certain G-proteins are still comparatively understudied in terms of their GPCR partners, particularly G_{12/13}, which signal principally through Rho GTPases. Moreover, for the majority of well-studied receptors, only the primary (i.e. the most prominent) coupling is known, with secondary couplings known only for a minority. Yet, this G-protein coupling information is limited to binary (primary coupling and not stated) or tertiary (primary, secondary couplings and not stated) scoring and fails to provide quantitative data sufficient to achieve successful bioinformatic analyses including GPCR residues involving G-protein coupling selectivity.

Efforts to predict coupling on the basis of sequence features have been made to complement the absence of a complete picture of G-protein signaling, especially for G_{12/13} coupling as well as orphan GPCRs (Sgourakis et al., 2005b; Yabuki et al., 2005). In case of G_{12/13}, owing to limited availability of signaling assays, coupling information on this class of G-proteins is incomplete. In addition, for orphan GPCRs, which lack pharmacological compounds to activate receptors, an accurate signaling prediction is desired to investigate not only coupling information, but also ligand identification to be investigated. Although many methods have been employed, previous researches generally sought to identify broad sequence properties at particular sites on the sequences that are indicative of a particular coupling subgroup. These methods have met with mixed success, and usually following poorer performances for G_{12/13} coupling prediction.

Despite many advances in the understanding of GPCRs, the mechanisms by which they specifically signal through G-proteins remain poorly understood.

Thus, there are still needs in the field of GPCR signalling to provide an improved method for determining a coupling probability between a G-protein and a G-protein coupled receptor (GPCR), an improved method for designing a G-protein coupled receptor (GPCR) with a predetermined G-protein coupling profile, and an alternative method for determining dissociation of a Gα subunit from Gβγ subunits of a G-protein in view of GPCR ligand induced interaction.

In particular for pharmaceutical drug development, there also exists a need in providing a GPCR, which is designed to be exclusively activated by a designer drug (Designer Receptor Exclusively Activated by Designer Drugs (DREADD)). In addition, there exists a need in providing an optimized amino acid sequence for determining coupling or no coupling between G-protein and G-protein coupled receptor (GPCR), preferably a DREADD, mediated by a GPCR ligand in a cell based assay.

### SUMMARY OF INVENTION

The aforementioned needs are met in part or all by means of the claimed inventive subject matter. Preferred embodiments are in particular described in the dependent claims, the detailed description, the sequence listing and/or the accompanying figures. The inventive aspects may comprise—in case it is reasonable for a person skilled in the art—any possible combination of the different preferred inventive embodiments as set out hereinafter including the detailed description, the experimental section, the sequence listing and/or the accompanied figures.

Accordingly, a first aspect of the invention relates to a computer-implemented method for determining a probability of coupling or no coupling between a G-protein and a G-protein coupled receptor (GPCR). In other words, the first aspect of the invention acts as a predictor for GPCR/G-protein couplings or no couplings. Therefore, the inventive method of the first aspect is synonymously referred to as (inventive) predictor, if not otherwise stated. The inventive predictor can be used for a host of biological and pharmaceutical applications.

The inventive predictor is improved over the prior art predictor of Sgourakis et al., 2005b; Yabuki et al., 2005, as it can—in addition to predicting the GPCR/G-protein coupling probability—also predict the no coupling propability. Furthermore, the inventive predictor shows an increased sensitivity of predicting the GPCR/G-protein coupling, in particular GPCR/G-protein coupling selectivity.

The method/predictor of the first inventive aspect comprises or consists of the following steps:

Method step A:
a. Providing amino acid sequence data and/or three dimensional (3D) structural data of
   i. one or more G-proteins and one or more GPCRs known to couple as a G-protein/GPCR complex and
   ii. one or more G-proteins and one or more GPCRs known to not couple as a G-protein/GPCR complex.

In other words, acid sequence data and/or three dimensional (3D) structural data, preferably acid sequence data and optionally three dimensional (3D) structural data of the one or more G-proteins and GPCRs according to i) and ii) are grouped into coupled and uncoupled G-protein/GPCR complexes.

According to a preferred embodiment of the method step a) the amino acid sequence data and/or 3D structural data of the G-protein is provided for at least part of one or more of G-protein sub-families Gₛ, G_{i/o}, G_{q/11}, and G_{12/13}, preferably at least part of the a subunit of one or more of G-protein sub-families Gₛ, G_{i/o}, G_{q/11}, and G_{12/13}. The provision of data for G-protein sub-families, in particular the a subunit of one or more of G-protein sub-families Gₛ, G_{i/o}, G_{q/11}, and G_{12/13} allows more precise prediction for G-preotein sub-families. In addition or alternatively, the the amino acid sequence data and/or 3D structural data of the GPCR is preferably at least provided for part of the amino acid sequence data and/or 3D structural data of Class A GPCRs, more preferably wherein the part of the Class A GPCRs comprises or consists of at least part of the amino acid sequence data and/or 3D structural data of
i. one or more of the seven transmembrane bundle (7TM) features, such as transmembrane bundle 1 (TM1), transmembrane bundle 2 (TM2), transmembrane bundle 3 (TM3), transmembrane bundle 4 (TM4), transmembrane bundle 5 (TM5), transmembrane bundle 6 (TM6), and transmembrane bundle 7 (TM7), more preferably TM3, TM5, and TM6, and/or
ii. one or more of extra 7TM features, such as N-terminal, one or more extracellular loops (ECL), one or more intracellular loops (ICL), and/or C-terminal region, more preferably intracellular loop 3 (ICL3), and C-terminal region.

The above amino acid sequence data and/or 3D structural data of the seven transmembrane bundle (7TM) or extra 7TM of GPCRs are relevant for interactions with the G-protein.

According to a further preferred embodiment of the present invention, the amino acid sequence data and/or three dimensional (3D) structural data for of step a) comprises i) at least for one given G-protein data set a set of data of two or more respective coupling GPCRs and/or two or more respective uncoupling GPCRs and/or ii) at least for one given GPCR data set a set of data of two or more respective coupling G-proteins and/or two or more respective uncoupling G-proteins. In other words, the preferred embodiment provides not only primary, but also secondary, tertiary etc. coupling G-protein/GPCR data. Such data provision increases the sensitivity of the inventive predictor.

Method step B:
b. Statistically aligning the amino acid sequence and/or the 3D structural data of the G-protein with the GPCR of the respective coupled or uncoupled G-protein/GPCR complex provided in step a) in order to determine one or more amino acid residues and/or one or more structural composition features found to be statistically significantly associated with a coupled G-protein/GPCR complex or with an uncoupled G-protein/GPCR complex, and statistically assigning a coupling or uncoupling probability to the determined amino acid residues and/or structural composition features.

In other words, the significantly aligned one or more amino acid residues and/or one or more structural composition features are grouped into coupled or uncoupled G-protein/GPCR complex groups.

As the statistically determined amino acid residues and/or structural composition features are statistically significantly associated with a coupled G-protein/GPCR complex or with an uncoupled G-protein/GPCR complex, amino acid residues and/or structural composition features not statistically significantly associated with a coupled G-protein/GPCR complex or with an uncoupled G-protein/GPCR complex are in general not used for statistically assigning the coupling or uncoupling probability. Furthermore, the assignment of the coupling or uncoupling probability in generally depends on the p-value; a p-value of greater or equal 0.5 assigns a coupling probability and a p-value of less than 0.5 assigns a uncoupling probability for the respectively determined amino acid residues and/or structural composition features.

According to a preferred embodiment, the statistical alignment and assignment of coupling or uncoupling probability according to step b) uses a Hidden Markov Model (HMM) profile, which in particular allows more sequences to be significantly identified.

Method step C:
c. Training a machine learning classifier using the coupling or uncoupling probabilities assigned to the one or more amino acid residues and/or one or more structural composition features of step b) in order to create a predictor for determining a probability of coupling or no coupling between a G-protein and a G-protein coupled receptor (GPCR).

In other words, the assigned coupling or uncoupling probabilities of method step b) are classified in step c) by comparing the probabilities in relation to each other.

As an example, a logistic regression is used in the machine learning prediction of step c).

According to a preferred embodiment, the training step c) comprises a step of comparing the statistical alignment and probability of coupling or uncoupling and assigning weight for coupling or uncoupling to the respective significant coupling and/or uncoupling amino acid residues and/or structural composition features, more preferably in relation with a respective coupled or uncoupled G-protein/GPCR complex. The advantage of assigning respective weights to the probabilities results in an increased selectivity of predicting coupling or uncoupling of query G-proteins or query GPCRs with corresponding coupling partners.

### Method Step D:

d. Providing an amino acid sequence and/or 3D structural data of a query GPCR and applying the trained machine learning predictor of step c) in order to determine a probability that the query GPCR couples or uncouples to a predetermined G-protein.

In other words, method step D relates to input data for the machine learning predictor of step c) comprising a query GPCR (synonym: GPCR of interest) and predicting in relation to the classification of probabilities a G-protein/GPCR profile, which means that the query GPCR couples or uncouples with a certain probability to the predetermined G-proteins.

### Method Step E:

e. Providing an amino acid sequence and/or 3D structural data of a query G-protein and applying the trained machine learning predictor of step c) in order to determine a probability that the query G-protein couples or uncouples to a predetermined GPCR.

In addition to method step D or alternative thereto, the inventive method relates to input data for the machine learning predictor of step c) comprising a query G-protein (synonym: G-protein of interest) and predicting in relation to the classification of probabilities a G-protein/GPCR profile, which means that the query GPCR couples or uncouples with a certain probability to the predetermined GPCR.

The inventive predictor can comprise—in case it is reasonable for a person skilled in the art—any possible feature combination of the different inventive embodiments including preferred and alternative features. Moreover, the embodiments of the inventive predictor can comprise—in case it is reasonable for a person skilled in the art—any possible feature combination with singular or combined features of embodiments disclosed in the detailed description, the experimental section, sequence listing and/or figures.

According to the second aspect of the present invention, a computer-implemented method for designing a G-protein coupled receptor (GPCR) with a predetermined G-protein coupling profile is provided. In other words, the second aspect of the invention acts as a designer for GPCR/G-protein couplings or no couplings. Therefore, the inventive method of the second aspect is synonymously referred to as (inventive) designer, if not otherwise stated. The inventive designer can be used for a host of biological and pharmaceutical applications.

The inventive designer is improved over the prior art predictor of Sgourakis et al., 2005b; Yabuki et al., 2005, as it can optimize the designed GPCR sequence in view of a predetermined G-protein/GPCR coupling profile and, thus, shows an increased sensitivity of designing a GPCR having a predetermined G-protein/GPCR coupling profile.

The method/designer of the second inventive aspect comprises or consists of the method steps a) to d) already disussed with respect to embodiments of the first aspect of the present invention, namely the predictor. All inventive embodiments including preferred features and feature combinations disclosed with respect to the first aspect of the present invention are also applicable to embodiments and preferred embodiments of the second aspect of the invention, namely the designer.

In addition thereto, the method/designer of the second inventive aspect method setp d) further comprises designing a GPCR with a predetermined G-protein coupling profile by amending the amino acid sequence and/or the 3D structural data of the query GPCR in order to optimize the probability that the GPCR couples to the predetermined G-protein and optionally to optimize the probability to not couple to other G-proteins.

In other words, the amino acid sequence and/or a 3D structural feature data of the query GPCR is optimized for a predetermined GPCR/G-protein coupling profile using the machine learning classifier of step c). According to a preferred embodiment the query GPCR is optimized for a predetermined GPCR/G-protein coupling profile of G-protein subfamilies in order to increase the sensitivity.

According to a further preferred embodiment, the inventive designer is used to design a Designer Receptor Exclusively Activated by Designer Drugs (DREADD).

The designer of the second aspect of the present invention can comprise—in case it is reasonable for a person skilled in the art—any possible feature combination of the different inventive embodiments.

According to a third aspect of the present invention, a computational data processing system is provided comprising data processing system having one or more processors coupled to a memory, having inputting and having outputting means. The data processing system of the third inventive aspect is configured to
a. determine a probability of coupling or no coupling between a G-protein and a G-protein coupled receptor (GPCR) according to any one of the feature combinations of the inventive predictor of the first aspect of the present invention, or
b. design a G-protein coupled receptor (GPCR) with a predetermined G-protein coupling profile according to any one of the feature combinations of the inventive designer of the second aspect of the present invention.

In general, the inventive system can at least in part be installed on a local server or on a webserver, in particular a cloud based webserver. An end user may use this inventive system via a suitable browser or software application to be downloadable on an end user device or another device connectable to the end user device.

The inventive data communication system can comprise—in case it is reasonable for a person skilled in the art—any possible feature combination of the different inventive embodiments including preferred and alternative features of the inventive predictor and inventive designer of the first and second inventive aspects, respectively. Moreover, the inventive embodiments can comprise—in case it is reasonable for a person skilled in the art—any possible feature combination with singular or combined features of embodiments disclosed in the detailed description, the experimental section, sequence listing and/or figures.

According to a fourth aspect of the present invention, the inventive predictor, the inventive designer and/or the inventive data processing system can be used together with one or more further data sets relating to the same or other GPCR signaling pathways selected from the group consisting of genomic sequencing, transcriptomics, proteomics, and/or metabolomics in quantification of GPCR downstream signaling in normal and/or pathological conditions.

The use of the fourth aspect of the present invention can comprise—in case it is reasonable for a person skilled in the art—any possible feature combination of the different inventive embodiments including preferred and alternative features of the inventive predictor, the inventive designer and the inventive data processing system of the first, second and third inventive aspects, respectively. Moreover, the inventive embodiments can comprise—in case it is reasonable for a person skilled in the art—any possible feature combination with singular or combined features of embodiments disclosed in the detailed description, the experimental section, sequence listing and/or figures.

According to a fifth aspect of the present invention, a Designer Receptor Exclusively Activated by Designer Drugs (DREADD) is provided, wherein the DREADD is a G-protein coupled receptor (GPCR). The DREADD may be obtainable by the inventive designer method according to the second aspect of the present invention. Such a designed DREADD is in particular relevant, as it can be designed for optimized G-protein sub-family coupling profile, preferably comprising a G₁₂—specific/GPCR coupling profile. According to one preferred embodiment the DREADD is a G₁₂—specific GPCR responding to a ligand and comprises or consists of an amino acid sequence according to SEQ ID Nos: 2, 3, or 4 or an amino acid sequence having at least 90, 91, 92, 93, 94, 95, 96, 97, 98, 99% sequence identity to SEQ ID Nos: 2, 3, or 4. The inventive DREADDs may be used also in other aspects of the present invention, such as in assays used for biologic and pharmaceutical developments, in particular the inventive assays as set out below in the sixth aspect of the present invention. The inventive DREADDs are in particular preferred when profiling a G₁₂ coupling to a GPCR.

The DREADD of the fifth aspect of the present invention can comprise—in case it is reasonable for a person skilled in the art—any possible feature combination of the different inventive embodiments including preferred and alternative features of the inventive predictor, the inventive designer and the inventive data processing system of the first, second and third inventive aspects, respectively. Moreover, the inventive embodiments can comprise—in case it is reasonable for a person skilled in the art—any possible feature combination with singular or combined features of embodiments disclosed in the detailed description, the experimental section, sequence listing and/or figures.

According to a sixth aspect of the present invention, a method for determining coupling or no coupling between G-protein and G-protein coupled receptor (GPCR) mediated by a GPCR ligand in a cell is provided. In other words, a cell or membrane based assay for determining a G-protein/GPCR coupling profile is provided. Therefore, the inventive method of the sixth aspect may synonymously be referred to as inventive assays. The inventive assay is characterized in that it uses a split luciferase complement system (NanoBIT). The NanoBIT system itself (a pair of large fragment of split luciferase (LgBiT) sequences and small fragment of split luciferase (SmBIT) sequences along with a 15-amino acid linker) was established by Promega (Dixon et al. ACS chemical biology 11, 400-408 (2016). PMID 26569370) and comprises the following sequences:
the large fragment of split luciferase (LgBIT) consists of an amino acid sequence according to SEQ ID No: 49
the small fragment of split luciferase (SmBIT) consists of an amino acid sequence according to SEQ ID No: 50
the flexible linker consists of an amino acid sequence according to SEQ ID No: 51

According to the present invention, in particular the inventive assays, the inventors generated LgBiT- or SmBiT-fused chimeric proteins as set out in more detail below and showed that these engineered chimeric proteins are useful for analyzing G protein activation in cells (and also in membrane preparation) and thereby determining coupling or no coupling between G-protein and G-protein coupled receptor (GPCR).

The inventive cell assay comprises or consists of the following assay method steps:

### Assay Method Step A:

a. Providing a dissociation cell assay of a Gα subunit from Gβγ subunits of a chimeric G-protein comprising the GPCR, wherein the chimeric G-protein is expressed in the cell comprising a large fragment of split luciferase (LgBiT), preferably inserted with a flexible linker amino acid sequence into the helical domain, more preferably between the αA and αB helices or αB and αC, of the Gα subunit of the chimeric G-protein and a small fragment of the split luciferase (SmBIT), preferably fused with a flexible linker amino acid sequence to an N-terminal region of the Gβ and/or Gγ subunit of the chimeric G-protein.

According to a preferred embodiment of the dissociation cell assay the chimeric G-protein subunits comprise or consist of the following sequences:
the Gα subunit of the chimeric G-protein comprises or consists of an amino acid sequence according to any one of SEQ ID Nos: 5 to 15 and 33 to 41 or an amino acid sequence having at least 90, 91, 92, 93, 94, 95, 96, 97, 98, 99% sequence identity to any one of SEQ ID Nos: 5 to 15 and 33 to 41, and/or
the Gβ subunit of the chimeric G-protein comprises or consists of an amino acid sequence according to any one of SEQ ID Nos: 16 to 20, and 42 or an amino acid sequence having at least 90, 91, 92, 93, 94, 95, 96, 97, 98, 99% sequence identity to any one of SEQ ID Nos: 16 to 20, and 42, and/or
the Gγ subunit of the chimeric G-protein comprises or consists of an amino acid sequence according to any one of SEQ ID Nos: 21 to 32, 43 and 44 or an amino acid sequence having at least 90, 91, 92, 93, 94, 95, 96, 97, 98, 99% sequence identity to any one of SEQ ID Nos: 21 to 32, 43 and 44.

### Or Assay Method Step B:

b. Providing a Ras homolog gene family, member A (RhoA) GTPase activation cell assay comprising chimeric RhoA GTPase and a chimeric PKC-related serine/threonine-protein kinase N1 (PKN1), wherein the chimeric RhoA GTPase is expressed in the cell comprising a large fragment of split luciferase (LgBiT), preferably fused with a flexible linker amino acid sequence to the N-terminal region of the chimeric RhoA GTPase, and wherein the PKN1 is expressed in the cell comprising a small fragment of the split luciferase (SmBIT), preferably fused with a flexible linker amino acid sequence to the N-terminal region.

The RhoA GTPase activation cell assay is in particular advantageous when determining the G_{12/13} G-protein subunit with GPCR. According to a further preferred embodiment of the RhoA GTPase activation cell assay the chimeric RhoA GTPase and/or chimeric PKN1 comprise or consist of the following sequences:
the chimeric RhoA GTPase comprises or consists of an amino acid sequence according to SEQ ID No: 55 or an amino acid sequence having at least 90, 91, 92, 93, 94, 95, 96, 97, 98, 99% sequence identity to SEQ ID No: 55, and/or
the chimeric PKC-related serine/threonine-protein kinase N1 (PKN1)
comprises or consists of an amino acid sequence according to SEQ ID No: 56 or an amino acid sequence having at least 90, 91, 92, 93, 94, 95, 96, 97, 98, 99% sequence identity to SEQ ID No: 56.

### Or Assay Method Step C:

c. Providing inositole triphosphate (IP3) accumulation cell assay comprising a chimeric inositole triphosphate receptor (IP3R), wherein the chimeric IP3R is expressed in the cell comprising a large fragment of split luciferase (LgBIT), preferably fused with a flexible linker amino acid sequence to the N-terminal region of the IP3R and comprising a small fragment of the split luciferase (SmBIT) spaced from the LgBiT, preferably fused with a flexible linker amino acid sequence to the C-terminal region of the IP3R.

According to a preferred embodiment of the IP3 accumulation cell assay the chimeric IP3R, preferably IP3R2 comprises or consists of the following sequence:
the chimeric inositole triphosphate receptor (IP3R) is based on the inositol triphosphate receptor 2 (IP3R2) and preferably comprises or consists of an amino acid sequence according to SEQ ID No: 57 or an amino acid sequence having at least 90, 91, 92, 93, 94, 95, 96, 97, 98, 99% sequence identity to SEQ ID No: 57, and/or

### OR Assay Method Step D:

d. Providing Gq and 1-phosphatidylinositol-4,5-bisphosphate phospholipase Cbeta (PLCβ) interaction cell assay comprising a chimeric Gα subunit from the Gαq family and a chimeric PLCβ, wherein the chimeric Gαq subunit is expressed in the cell comprising a large fragment of split luciferase (LgBiT), preferably inserted with flexible linker amino acid sequences into the helical domain, more preferably between the αA and αB or αB and αC helices, of the Gαq subunit of the chimeric G-protein, and wherein the chimeric PLCβ is expressed in the cell comprising a small fragment of the split luciferase (SmBIT), preferably fused with a flexible linker amino acid sequence to the N-terminal region.

The Gq-PLCβ interaction assay is in particular advantageous when determining the Gq/11 G-protein subunit with GPCR. According to another preferred embodiment of the Gq-PLCβ interaction cell assay the chimeric Gαq subunit and/or chimeric PLCβ comprise or consist of the following sequences:
the chimeric Gα subunit comprises or consists of an amino acid sequence according to SEQ ID No: 10-13 and 38-41 or an amino acid sequence having at least 90, 91, 92, 93, 94, 95, 96, 97, 98, 99% sequence identity to SEQ ID No: 10-13 and 38-41, and/or
the chimeric PLCβ comprises or consists of an amino acid sequence according to SEQ ID No: 45-48 or an amino acid sequence having at least 90, 91, 92, 93, 94, 95, 96, 97, 98, 99% sequence identity to SEQ ID No: 45-48.

### And

### Assay Method Step E:

e. Contacting the cell of assay method step a), b), c) or d) with a luciferase substrate.

In other words, the cells comprised in the respective assays of method steps a), b) or c) are loaded with a suitable amount of luciferase. As preferred embodiment, the luciferase substrate is coelenterazine (CTZ) or a comparable luciferase substrate.

The associated chimeric G-protein subunits of the inventive G-protein dissociation assay (Assay Method Step A) form a bioluminescence active construct in presence of a luciferase substrate.

The dissociated chimeric RoA GTPase and chimeric PKN1 of the inventive RhoA GTPase activation assay (Assay Method Step B) are bioluminescence inactive in presence of a luciferase substrate.

The chimeric IP3R of the inventive IP3 accumulation cell assay (Assay Method Step C) is as such bioluminescence inactive in presence of a luciferase substrate.

The associated chimeric Gαq subunit and the chimeric PLCβ protein of the inventive Gq-PLCβ interaction assay form a bioluminescence active construct.

### And Assay Method Step F:

f. Contacting the cell of step e) with a ligand of the GPCR.

In other words, the cells of the inventive assays are incubated with a suitable ligand for each assay in order to activate the signaling pathway of the respective GPCR.

In case a suitable GPCR ligand binds to the GPCR of the inventive G-protein dissociation assay, coupling of the GPCR with the Gα subunit of the chimeric G-protein is mediated and dissociation of the Gα subunit from Gβγ subunits of the chimeric G-protein is initiated. Upon dissociation of the Gα subunit from Gβγ subunits of the chimeric G-protein guanosine diphosphate (GDP) is released from the Gα subunit and guanosine triphosphate (GTP) is bound to the Gα subunit. The dissociated chimeric G-protein is bioluminescence inactive (see also FIG. 11A).

In case a suitable GPCR ligand binds to the GPCR of the inventive RhoA GTPase activation cell assay, activation of RhoGTPase nucleotide exchange factors (RhoGEFs) is mediated. Upon activation of the RhoGEFs GDP is released from the chimeric RhoA GTPase and GTP is bound thereto. This exchange facilitates the coupling of the chimeric RhoA GTPase and the chimeric PKN1. Upon coupling of the chimeric RhoA GTPase and the chimeric PKN1 the LgBIT and SmBIT form a bioluminescent active construct (see also FIG. 13A).

In case a suitable GPCR ligand binds to the GPCR of an IP3R activation cell assay, 1-phosphatidylinositol-4,5-bisphosphate phosphodiesterase beta (PLCβ) is activated mediating the coupling of IP3 with IP3R, preferably IP3R2. Upon coupling, the SmBIT and LgBiT fragments associate to form a bioluminescence active construct (see also FIG. 14D).

In case a suitable GPCR ligand binds to the GPCR of an Gq-PLCβ interaction cell assay, a chimeric Gα subunit from the Gαq family interacts with the chimeric 1-phosphatidylinositol-4,5-bisphosphate phosphodiesterase beta (PLCβ). Upon binding, the SmBIT and LgBiT fragments associate to form a bioluminescence active construct (see also FIG. 16P).

### And Assay Method Step G:

g. Measuring a bioluminescence signal of the cell of step f) and optionally the cell of step e).

The biolouminescence signal is measured in step f) of the inventive assays, wherein the bioluminescence signal corresponds to the formation of associated LgBiT and SmBIT fragments. Optionally the background fluorescence in step e) is additionally measured. Alternatively, reference fluorescence data may be provided in order to carry out step h).

### And Assay Method Step H:

h. determining coupling or no coupling between G-protein and G-protein coupled receptor (GPCR) as a function of the measured bioluminescence signal in step f).

In other words, the higher the delta of signals measured in steps e) and f), the higher the probability of coupling. A threshold value may be used. Alternatively, the bioluminescence signal measured in step f) may be compared to an external reference signal value.

The inventive cell assays can comprise—in case it is reasonable for a person skilled in the art—any possible feature combination of the different inventive embodiments including preferred and alternative features. Moreover, the embodiments of the inventive cell assays can comprise—in case it is reasonable for a person skilled in the art—any possible feature combination with singular or combined features of embodiments disclosed in the detailed description, the experimental section, sequence listing and/or figures.

According to a seventh aspect of the present invention an amino acid sequence for determining coupling or no coupling between G-protein and G-protein coupled receptor (GPCR) mediated by a GPCR ligand in a cell based assay is provided, characterized in that the amino acid sequence is selected from
a Gα subunit of the chimeric G-protein, which comprises or consists of an amino acid sequence according to any one of SEQ ID Nos: 5 to 15 and 33 to 41 or an amino acid sequence having at least 90, 91, 92, 93, 94, 95, 96, 97, 98, 99% sequence identity to any one of SEQ ID Nos: 5 to 15 and 33 to 41, and/or
a Gβ subunit of the chimeric G-protein, which comprises or consists of an amino acid sequence according to any one of SEQ ID Nos: 16 to 20, and 42 or an amino acid sequence having at least 90, 91, 92, 93, 94, 95, 96, 97, 98, 99% sequence identity to any one of SEQ ID Nos: 16 to 20, and 42, and/or
a Gγ subunit of the chimeric G-protein, which comprises or consists of an amino acid sequence according to any one of SEQ ID Nos: 21 to 32, 43 and 44 or an amino acid sequence having at least 90, 91, 92, 93, 94, 95, 96, 97, 98, 99% sequence identity to any one of SEQ ID Nos: 21 to 32, 43 and 44, and/or
a chimeric RhoA GTPase, which comprises or consists of an amino acid sequence according to SEQ ID No: 55 or an amino acid sequence having at least 90, 91, 92, 93, 94, 95, 96, 97, 98, 99% sequence identity to SEQ ID No: 55, and/or
a chimeric PKC-related serine/threonine-protein kinase N1 (PKN1), which comprises or consists of an amino acid sequence according to SEQ ID No: 56 or an amino acid sequence having at least 90, 91, 92, 93, 94, 95, 96, 97, 98, 99% sequence identity to SEQ ID No: 56, and/or
a chimeric inositole triphosphate receptor (IP3R), which is based on the inositol triphosphate receptor 2 (IP3R2) and comprises or consists of an amino acid sequence according to SEQ ID No: 57 or an amino acid sequence having at least 90, 91, 92, 93, 94, 95, 96, 97, 98, 99% sequence identity to SEQ ID No: 57 and/or
the chimeric 1-phosphatidylinositol-4,5-bisphosphate phospholipase Cbeta PLCβ, which comprises or consists of an amino acid sequence according to SEQ ID Nos: 45 to 48 or an amino acid sequence having at least 90, 91, 92, 93, 94, 95, 96, 97, 98, 99% sequence identity to SEQ ID Nos: 45 to 48.

Further aspects of the present invention relate to an inventive TGFα shedding assay, Active RhoA pulldown assay, Ca²⁺ mobilization assay.

### BRIEF DESCRIPTION OF DRAWINGS

Further aspects, characteristics and advantages of the invention will ensue from the following description of the embodiments with reference to the accompanying drawings, wherein

FIG. 1 relates to chimeric G-protein-based TGFα shedding assay to probe interaction between an active GPCR and a C-terminal tail of a Gα subunit, wherein

FIG. 1(A) represents a schematic description of the mechanism of the TGFα shedding assay.

FIG. 1(B) represents graphs of blunted TGFα shedding response in the HEK293 cells devoid of the Gq/11 and the G_{12/13} subfamilies.

FIG. 1(C) represents schematic description of the chimeric G-protein-based TGFα shedding assay in ΔG_{q}/ΔG₁₂ cells.

FIG. 1(D) represents an overview on representative data for the chimeric G-protein-based assay.

FIG. 2 represents signatures of G-protein coupling determined by the chimeric G-protein-based assay.

FIG. 3 relates to a comparison between dataset of the chimeric G-protein-based assay and GtoPdb and validation of G_{12/13} signaling for the newly characterized GPCRs, wherein

FIG. 3(A) represents a schematic classification of the LogRAi scores and its comparison with GtoPdb.

FIG. 3(B) represents a schematic view on combined binary coupling/non-coupling data for each of the four G-protein subfamilies.

FIG. 3(C) represents Venn diagrams with the numbers of receptors coupled to each G-protein subfamily in the chimeric G-protein-based assay (LogRAi≥−1).

FIG. 3(D) represents Venn diagrams of receptor couplings to the four G-protein families according to the chimeric G-protein-based assay (LogRAi≥−1) and GtoPdb.

FIG. 3(E) represents GPCRs that were identified as being coupled with G_{12/13} by the chimeric G-protein-based assay were examined for their ability to engage and activate native, endogenous G_{12/13} in HEK293 cells.

FIG. 4 relates to development of G-protein coupling predictor, wherein

FIG. 4(A) represents a schematic workflow of the procedure: features are extracted from sub-alignments of coupled and uncoupled receptors to a particular G-protein; features are used to generate a training matrix which is employed to train a logistic regression model through a 5-fold cross validation procedure.

FIG. 4(B) represents a schematic overview on the final model tested on reported couplings not previously seen during training and compared to PredCouple.

FIG. 4(C) represents graphs on highly confident predicted couplings (coupling probability >0.9) for 61 Class A GPCRs lacking information about transduction from both GtoPdb or the chimeric G-protein-based TGFα shedding assay (black) vs. receptors with experimental coupling information (gray).

FIG. 5 relates to featured residues in GPCRs involved in G-protein coupling selectivity, wherein

FIG. 5(A) represents a schematic overview on comparison of significant coupling features weights for the 11 G-proteins (bottom), interface contacts of 6 available GPCR-G-protein complexes (central) and 7TM domain position conservation (top).

FIG. 5(B) represents a schematic overview of the 7TM topology indicating the regions contributing to the features.

FIG. 5(C) represents a schematic overview on significant coupling feature weights for the 11 G-proteins (same color codes as in A) of extra-7TM features of ICL3 and C-term, including length and amino-acid composition.

FIG. 6 relates to a functional analysis of residues linked to coupling selectivity, wherein

FIG. 6(A) Upper panel represents: distribution of coupling feature fractions for intra- and extra-7TM portions. Lower panel represents: distribution of the coupling feature fractions within transmembrane sectors (i.e. extracellular-EC, transmembrane-TM, intracellular-IC).

FIG. 6(B) represents a graph on distribution of the fractions of coupling significant features outside of the 7TM bundle.

FIG. 6(C) represents a graph on distribution of coupling feature fractions (relative to the total number of positions of the same class) within functional sites (i.e. mediating either ligand/G-protein binding or intra-molecular contacts).

FIG. 6(D) represents a graph on intra-molecular contacts within 7TM helices.

FIG. 6(E) represents a three dimensional schematic view of the ADRB2-GNAS complex (PDB: 3SN6) with side chains of coupling features at G-protein-binding sites depicted as red surfaces.

FIG. 7 relates to the generation of G₁₂-coupled designer GPCRs, wherein

FIG. 7(A) represents a schematic view of generating and assessing ICL3- or ICL3/C-terminus-swapped constructs from G_{q/11}-coupled M3D.

FIG. 7(B-D) represent screening graphs of M3D-derived chimeric constructs.

FIG. 7(E) represents graphs on lack of G₁₃ activation by the new DREADD constructs.

FIG. 7(F) represents graphs on concentration-response curves for G-protein activation by DREADD constructs.

FIG. 8 relates to point mutation to enhance G-protein-coupling selectivity.

FIG. 9 relates to validation of the TGFα shedding assay, wherein

FIG. 9(A) represents graphs on siRNA-mediated knockdown of mRNA expression.

FIG. 9(B) represents an overview siRNA-mediated knockdown at protein expression levels.

FIG. 9(C) represents graphs on knockdown of G_{q/11} attenuates AP-TGFα release induced by G_{q/11}-coupled HRH1.

FIG. 9(D) represents graphs Knockdown of G_{12/13} attenuates AP-TGFα release induced by G_{12/13}-coupled PTGER3.

FIG. 9(E) represents graphs on parental, ΔG_{q}, ΔG₁₂ and ΔG_{q}/ΔG₁₂ HEK293 cells were transiently transfected with a plasmid encoding N-terminally FLAG epitope-tagged GPCR (HRH1, ADRB1 or DRD1) or an empty plasmid (Mock).

FIG. 9(F) represents cell surface expression levels dependent on C-terminal region of a co-expressed chimeric Gαq subunit.

FIG. 9(G) represents graphs on concentration-response curves of AGTR1 for the endogenous ligand (Angiotensin II, AngII) and a biased agonist ([Sar¹-IIe⁴-IIe⁸] AngII, SII). G-protein signaling activity was assessed by the chimeric-G-protein-based assay.

FIG. 9(H) represents ligand bias plots.

FIG. 9(I) represents graphs on validation of SII bias toward G₁₂ by the NanoBiT-G-protein dissociation assay.

FIG. 10 relates to chimeric Gα subunits and their activity for TGFα shedding and CAMP responses, wherein

FIG. 10(A) represents an overview on seven C-terminal sequences (CGN numbering (Flock et al., 2015) of G.H5.20-G.H5.26) of Gα subunits among the 16 human Gα subunits.

FIG. 10(B) represents an overview on evolutionally conservation the seven C-terminal sequences of representative Gα subunits from the four G-protein subfamilies.

FIG. 10(C) represents an overview on Chimeric G-proteins used in this study.

FIG. 10(D) represents graphs Capacity of Gα subunits to induce TGFα shedding response.

FIG. 10(E) represents graphs Capacity of Gα subunits to induce TGFα shedding response.

FIG. 10(F) represents an overview Protein expression levels of chimeric Gαq subunits.

FIG. 10(G) represents graphs on kinetics of cAMP level upon Gₛ-coupled receptor stimulation.

FIG. 10(H) represents graphs on concentration-response curves.

FIG. 10(I) represents an overview on chimeric G-protein-based CAMP assay in ΔGₛ cells.

FIG. 10(J) represents graphs on representative data for the chimeric G-protein-based cAMP assay.

FIG. 10(K) represents graphs on comparison of the chimeric G-protein backbones in coupling profiles.

FIG. 11 relates to development and validation of the NanoBiT-G-protein dissociation assay, wherein

FIG. 11(A) represents a schematic view of the NanoBiT-G-protein assay.

FIG. 11(B) represents a graph on luminescent kinetics of NanoBiT-G-proteins after GPCR ligand stimulation.

FIG. 11(C-E) represent graphs on the effect of preincubation time with CTZ

FIG. 11(F) represents graphs on validation of the NanoBiT-G-proteins by using prostanoid receptors.

FIG. 11(G) represents graphs on comparison of coupling profiling between the chimeric G-protein-based TGFα shedding assay and NanoBiT-G-protein assay.

FIG. 12 relates to analysis of the chimeric G-protein-based assay dataset and comparison with GtoPdb, wherein

FIG. 12(A) represents a graph on Roc curve comparing the chimeric G-protein-based TGFα shedding assay couplings with GtoPdb couplings.

FIG. 12(B) represents a graph on number of GPCRs coupled to G-proteins of the four families at different LogRAi thresholds in the chimeric G-protein-based TGFα shedding assay as well as in GtoPdb.

FIG. 12(C) represents a graph on distribution of the number of reported bindings (of any of the four G-protein families) for each receptor at different LogRAi thresholds in the chimeric G-protein-based TGFα shedding assay as well as in GtoPdb.

FIG. 12(D-F) represent an overview on fractions of specific couplings.

FIG. 12(G) represents a graph on comparison of receptor sequence and coupling profile similarities.

FIG. 13 relates to validation of RhoA activation by the newly identified G_{12/13}-coupled GPCRs, wherein

FIG. 13(A) represents a schematic view of the NanoBiT-RhoA sensor.

FIG. 13(B) represents graphs on luminescent kinetics of the NanoBiT-RhoA sensor after GPCR ligand stimulation.

FIG. 13(C) represents graphs on validation of G_{12/13}-mediated signal of the NanoBiT-RhoA sensor.

FIG. 13(D) represents graphs on NanoBiT-RhoA activation by selected GPCRs.

FIG. 13(E) represents graphs on NanoBiT-RhoA activation through endogenously expressed GPCRs.

FIG. 13(F) represents an overview on RhoA pulldown assay to detect G_{12/13} activation by endogenously expressed GPCRs.

FIG. 14 relates to G_{q/11} signaling in the absence of G_{12/13} for GPCRs coupled with G_{q/11} and G_{12/13}, wherein

FIG. 14(A) represents an overview on protein expression levels of Gα subunits.

FIG. 14(B) represents an overview on parameters obtained from concentration-response curves of the chimeric G-protein-based TGFα shedding assay.

FIG. 14(C) represents graphs on Ca²⁺ mobilization assay.

FIG. 14(D) represents a schematic overview of the NanoBiT-IP3 sensor.

FIG. 14(E) represents graphs on luminescent kinetics of the NanoBiT-IP3 sensor after GPCR ligand stimulation.

FIG. 14(F) represents a graph on validation of G_{q/11}-mediated signal of the NanoBiT-IP3 sensor.

FIG. 14(G) represents a graph on measurement of IP3 formation in EDNRA.

FIG. 15 relates to predictor performances, shortest path from contact network analysis, DREADD predictions scatter plot, wherein

FIG. 15(A) represents a radial plot representing Matthew correlation coefficient (MCC) of 5-fold cross validation (averaged over 10 runs).

FIG. 15(B) represents a radial plot representing Recall (Sensitivity) of the best performing predictors over the Test set.

FIG. 15(C) represents a bar plot representing the recall (sensitivity) of the best performing predictors, trained at different LogRAi cutoffs, over the test set.

FIG. 15(D) represents an overview on example of a shortest communication pathway, depicted on 3D cartoons of the ADRB2-GNAS complex (PDB ID: 3SN6), linking the ligand and G-protein consensus binding pocket pockets.

FIG. 15(E) represents a connectivity matrix displaying shortest paths (as intersecting circles) linking residues forming the ligand and G-protein consensus binding pockets (i.e. shown to form such interfaces in at least 50% of the considered structures).

FIG. 15(F) represents a scatter plot of the relative coupling probabilities of chimeric sequences obtained by swapping on the hM3D backbone sequence the sequence stretches corresponding to the ICL3 alone (y-axis) or in combination with the C-term (x-axis) from the 148 receptors of the chimeric G-protein-based TGFα shedding assay.

FIG. 16 relates to validation and application of the NanoBiT-G-proteins, wherein

FIG. 16(A) represents a heatmap representing G-protein dissociation profiles across Gβ and Gγ subtypes.

FIG. 16(B-E) represents membrane-based NanoBiT-G-protein dissociation assay.

FIG. 16(F-I) represents robustness of the NanoBiT-G-protein dissociation assay.

FIG. 16 (J-K) represents assessment of G-protein inhibitor.

FIG. 16 (L-N) represents enhanced sensitivity of the NanoBiT-G-protein dissociation assay.

FIG. 16 (O) represents assessment of NanoBiT-G-protein constructs.

FIG. 16 (P) represents schematic view e to assess NanoBiT-G-protein activation by its interaction with PLCβ (NanoBiT-G_{q}/PLC assay).

FIG. 16 (Q) represents graphs on luminescent kinetics of NanoBiT-G_{q}/PLC assay after GPCR ligand stimulation.

FIG. 16 (R) represents combinations of the Gα_{q} family members and PLCβ subtypes.

FIG. 16(S-T) represents detection of CaSR activation by Ca²⁺.

### DETAILED DESCRIPTION OF EMBODIMENTS

Following the general description of the inventive aspects in the summary of the invention, detailed aspects of the inventive embodiments are discussed below in detail. Combination of a singular feature of different feature combinations of the detailed description may be inventively combined with other general features or feature combinations as set out in the summary of the invention.

G_{q/11}- and G_{12/13}-dependent TGFα Shedding Responses

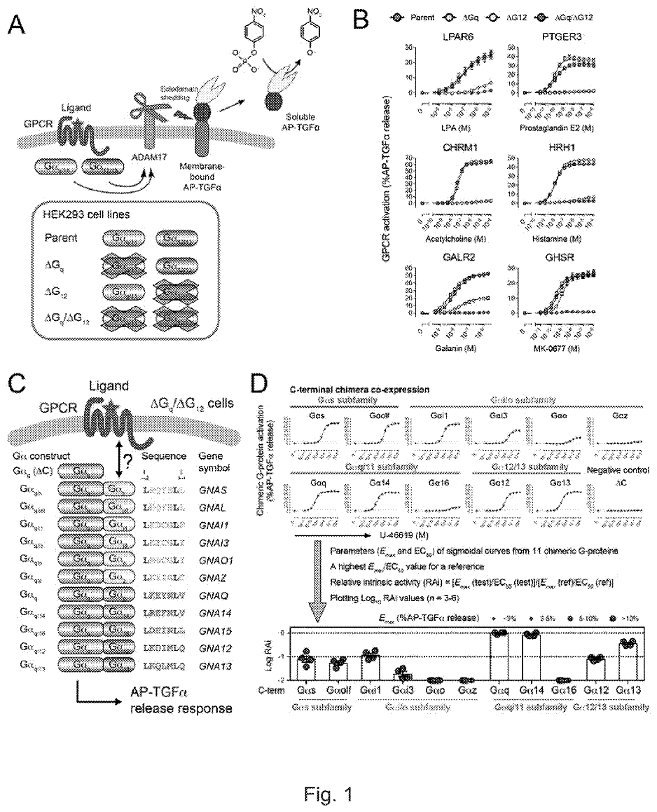

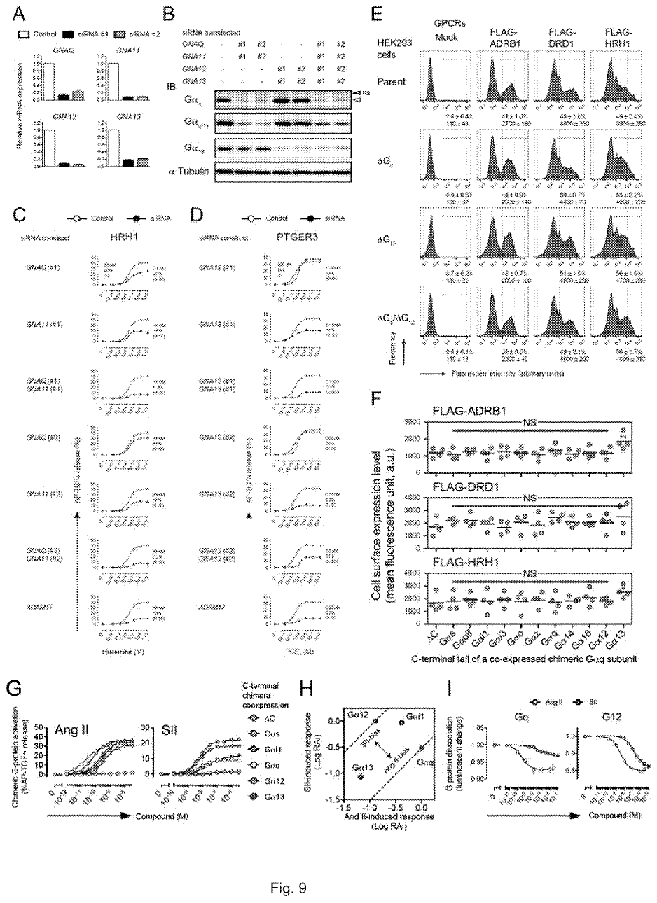

To evaluate G-protein coupling, the inventors exploited a TGFα shedding assay (FIG. 1A), which they showed previously to be a robust, high-throughput means to measure accumulated GPCR signals (Inoue et al., 2012). In the assay, they detect ADAM17-induced ectodomain shedding of alkaline phosphatase-fused TGFα (AP-TGFα) and subsequent release into conditioned media. The inventors previously observed that G_{q/11}- or G_{12/13}-coupled receptors induce this process (Inoue et al., 2012), which they first tested using a panel of HEK293 cells lacking one or both of the G_{q/11} and the G_{12/13} subfamilies (hereafter denoted as ΔG_{q}, ΔG₁₂ and ΔG_{q}/ΔG₁₂; FIG. 1A) (Devost et al., 2017; Schrage et al., 2015). The inventors tested GPCRs (FIG. 1B) that are reported to couple with either G_{q/11} (CHRM1 and HRH1 (Harding et al., 2018)) or G_{12/13} (LPAR6 and PTGER3 (Kihara et al., 2014; Sugimoto and Narumiya, 2007)) or both (GALR2 and GHSR (Harding et al., 2018)). TGFα shedding responses of the G_{q/11}-coupled receptors and the G_{12/13}-coupled receptors were diminished in ΔG_{q} and ΔG₁₂ cells, respectively, while the responses were retained in cells lacking uncoupled G-proteins. In the receptors coupling to both, the TGFα shedding responses remained in ΔG_{q} and ΔG₁₂ cells. For all tested GPCRs, TGFα shedding responses were completely abolished in ΔG_{q}/ΔG₁₂ cells nor could G₈⁻ or G_{i/o}-coupled receptors induce TGFα shedding responses. siRNA-mediated knockdown experiments in the parental HEK293 cells confirmed involvement of G_{q/11} and G_{12/13} in the TGFα shedding response (FIG. 9). Thus, this is clear evidence that the TGFα shedding assay selectively measures G_{q/11} and/or G_{12/13} signaling.

To exclude the possibility that the blunted AP-TGFα release signal was caused by loss of GPCR expression, the inventors compared surface expression levels of epitope-tagged GPCRs among parental, ΔG_{q}, ΔG₁₂ and G_{q}/ΔG₁₂ cells using a flow cytometry. All tested GPCRs (FLAG-ADRB1, FLAG-HRH1 and FLAG-DRD1) were equally expressed in the parental as well as the G-protein-KO cells (FIG. 10A).

### Chimeric G-Protein-Based Signaling Assay

The inventors exploited the above assay system, and the previously identified importance of the Gα subunit C-terminus, to develop the inventive TGFα shedding assay to assess binding of G-proteins to any GPCR of interest (query GPCR). Specifically, they constructed chimeric Gα subunits where the native 6-amino acid C-termini of members from the Gα_{q/11} and the Gα_{12/13} families were substituted with those from other human Gα subunits (FIGS. 1C and FIGS. 10A-C and SEQ ID No. 58 to 92) and expressed them together with a test GPCR in the signaling-silenced ΔG_{q}/ΔG₁₂ cells (FIG. 1C). The resulting downstream signals measured by the TGFα shedding assay should thus reflect the true binding events between any GPCR and its G-protein counterparts (FIG. 1D).

The inventors tested a series of chimeric Gα subunits for their ability to induce the TGFα shedding response. Specifically, they constructed chimeric Gα subunits with the same C-terminal tail, but a different backbone (FIGS. 10D-E), using members of the G_{q/11} (Gα_{q}, Gα₁₁, Gα₁₄ and Gα₁₆ subunits) and the G_{12/13} (Gα₁₂ and Gα₁₃ subunits) subfamilies. They expressed each chimeric Gα subunits (C-terminal Gai or Gαₛ chimeras) together with a test/query GPCR (G_{i/o}-coupled DRD2 or Gₛ-coupled PTGER2, respectively) and stimulated the cells with an agonist. The inventors found that the Gα_{q} backbone was the most efficacious in inducing TGFα shedding response (% AP-TGFα release response) for both receptors (FIGS. 10D-E); they thus chose this backbone for all subsequent experiments.

The inventors generated chimeric Gα_{q} subunits for each of the 11 unique C-terminal hexapeptides, which cover all of the 16 human Gα subunits (FIGS. 1C, 10A-C; C-terminal 6-amino acids are identical for Gαᵢ₁, Gαᵢ₂, Gαₜ₁, Gαₜ₂ and Gαₜ₃; and for Gα_{q} and Gα₁₁), and one negative control lacking the tail (Gα_{q} ΔC). The 11 Gα_{q} chimeras were equally expressed in ΔG_{q}/ΔG₁₂ cells (FIG. 10F). Transfected cells were harvested and seeded in a 96-well plate and stimulated with or without titrated concentrations of a GPCR ligand (typically, 12 points in total). AP-TGFα release signals over titrated concentrations were fitted with a sigmoidal concentration-response curve, from which EC₅₀ and Eₘₐₓ (an amplitude of ligand-induced response) values were obtained. For each chimeric Gα condition, an Eₘₐₓ/EC₅₀ value was normalized by the maximum Eₘₐₓ/EC₅₀ value among the 11 Gα chimeras (e.g., Gα_{q} C-terminus for TBXA2R; FIG. 1D). This gives a relative, dimensionless Eₘₐₓ/EC₅₀ value (relative intrinsic activity, RAi (Ehlert et al., 1999)), which is a base-10 log-transformed (LogRAi) and used as coupling indices. With their pre-determined threshold criteria (see section EXPERIMENTAL MODEL AND SUBJECT DETAILS), LogRAi ranged from −2 to 0 (100-fold in linear range). The assay produced robust, reproducible results as evidenced by well clustered plots across independent experiments (FIG. 1D). By using a similar approach (restoration of a chimeric Gα subunit in G-protein-KO cells), they performed a Gₛ-based cAMP assay and confirmed that LogRAi values obtained from the Gαₛ backbone and the Gog backbone were well correlated in prostanoid receptors (FIGS. 10I-K; r²=0.74±0.16, n=7), which show distinct G-protein-coupling profiles (Sugimoto and Narumiya, 2007; Woodward et al., 2011).

As above, the inventors measured cell surface expression of GPCRs by flow cytometry to exclude GPCR expression level effects (FIG. 9F). Except for a modest increase in the conditions with the chimeric Gα_{q/13} co-expression, expression levels of N-terminal FLAG epitope-tagged GPCRs (FLAG-ADRB1 and FLAG-HRH1) were almost equal among cells co-expressing any of the G□ chimeras.

### The NanoBiT-G-Protein Dissociation Assay

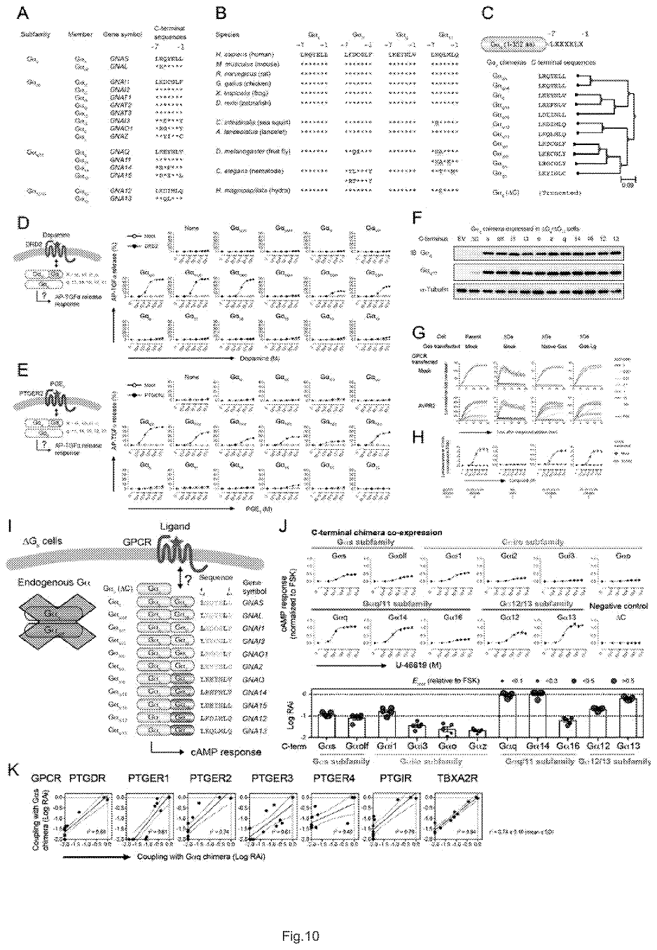

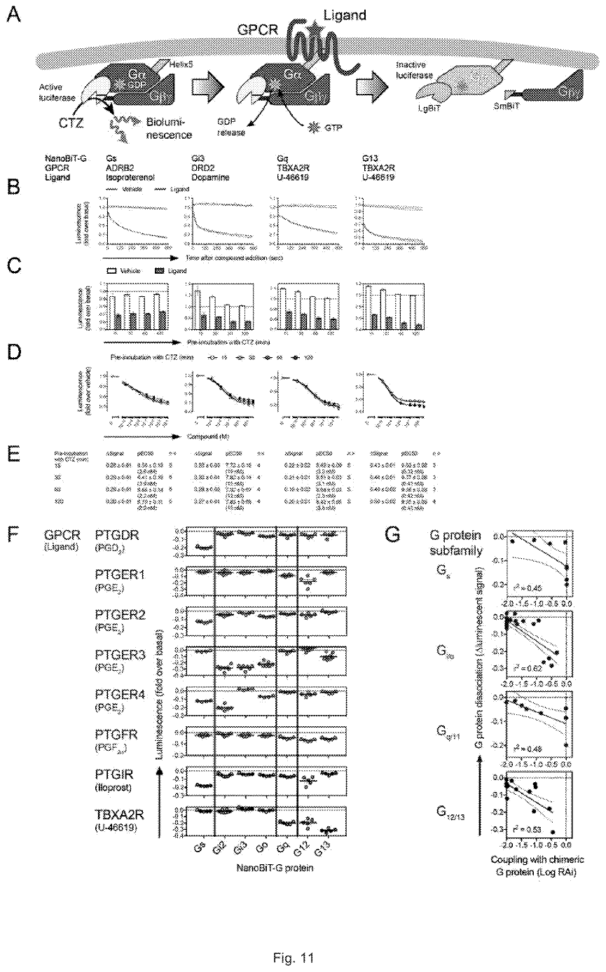

To complement the inventive chimeric G-protein-based TGFα shedding assay, the inventors made an additional inventive assay in which dissociation of the Gα subunit from the Gβγ subunits, a critical process of G-protein activation, is measured via a luciferase complementation system. Bioluminescence Resonance Energy Transfer (BRET) between a Renilla luciferase-inserted Gα subunit and a GFP10-fused Gβ or Gγ subunit was previously developed to measure Ga-Gβγ dissociation (Gales et al., 2005). Here, they replaced the BRET pair with a split luciferase (NanoLuc Binary Technology; NanoBiT) (Dixon et al., 2016). Specifically, they inserted a large fragment (LgBiT) of the NanoBiT into the helical domain (between the αA and αB helices) of a Gα subunit (Gα-Lg) and fused a small fragment (SmBIT) to the N-termini of Gβ or Gγ subunits (Sm-Gβ or Sm-Gγ) (see also SEQ ID. Nos: 5 to 51). The inventors confirmed that Gαₛ-Lg retained a Gₛ signaling function by measuring adenyl cyclase-activating activity upon a Gₛ-coupled receptor stimulation (FIGS. 10G-H). They generated a series of Gα-Lg, Sm-Gβ and Sm-Gγ subunits and optimized a combination. When expressed together in cells, these constructs form a heteromer with an enzymatically active luciferase, whose activity is measurable upon loading with coelenterazine (CTZ), a substrate of the luciferase (FIG. 11A). GPCR ligand stimulation triggers dissociation of Gα-Lg from Sm-Gβ/Gγ making the real-time dissociation response detectable (FIG. 11B). The inventive NanoBiT-G-protein assay demonstrated highly reproducible dissociation signals across independent experiments and was minimally affected by preincubation time with CTZ (FIG. 11F). Comparison of NanoBiT-G-protein dissociation signals with the chimeric G-protein-based assay for eight prostanoid receptors showed a moderately strong correlation (r²≥0.5) across all of the four G-protein subfamilies (FIGS. 11F-G).

### Ligand Biased G-Protein Signaling

Since the chimeric G-protein-based assay recognizes a ligand-activated conformation of a GPCR, the inventors assessed whether it could also detect ligand bias among different G protein subfamilies. An angiotensin II (Ang II) analog, [Sar¹, IIe^{4,8}]-Angiotensin II (SII), was shown to induce G^{i/o} over G_{q/11} as compared with Ang II in cells expressing AGTR1 (Sauliere et al., 2012). They performed the assay using Ang II and SII (FIG. 9G) and calculated coupling scores for Ang II-induced LogRAi and SII-induced LogRAi (FIG. 9H). If SII behaves as a balanced agonist across G-proteins, LogRAi plots obtained from Ang II and SII would be linearly aligned. their results recapitulated the Gᵢ-bias of SII (Sauliere et al., 2012), and further showed that SII was biased toward G₁₂ over G_{q} as compared with the reference ligand (Ang II). These findings were backed up by the NanoBiT-G-protein assay (FIG. 9I).

### Hundreds of Known and New Couplings

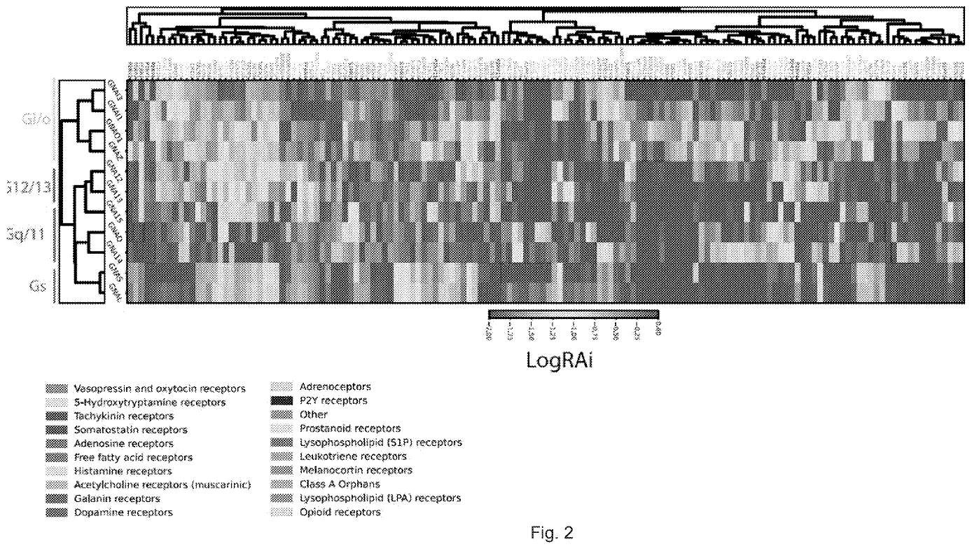

Using the inventive chimeric G-protein-based assay, the inventors profiled coupling across 148 human GPCRs (FIG. 2), which represent ~80% of liganded Class A GPCRs. Whenever possible, they used endogenous ligands; when ligands were unstable (e.g., thromboxane A2 for TBXA2R) and/or endogenous ligands were not yet identified, they chose available synthetic ligands (U-46619 for TBXA2R and MDL29951 for GPR17).

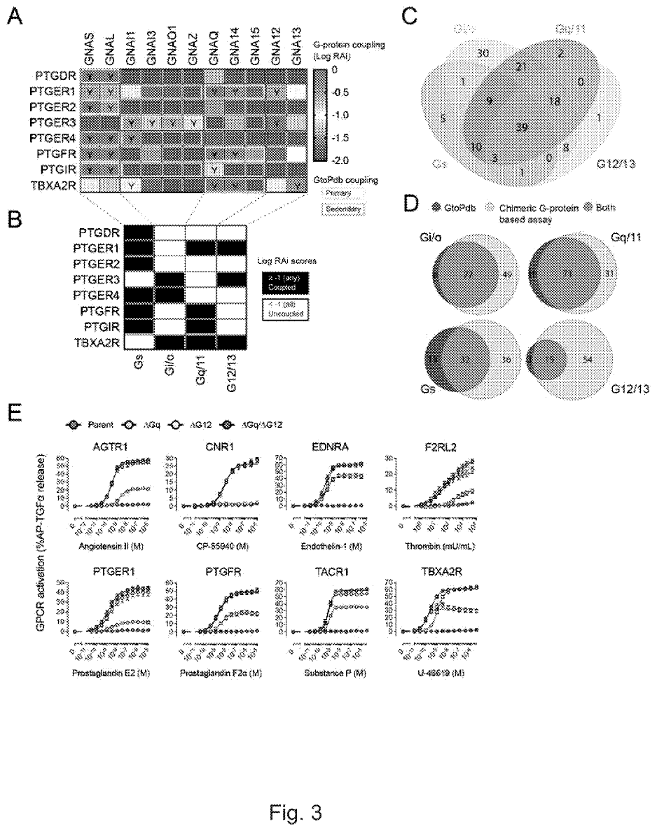

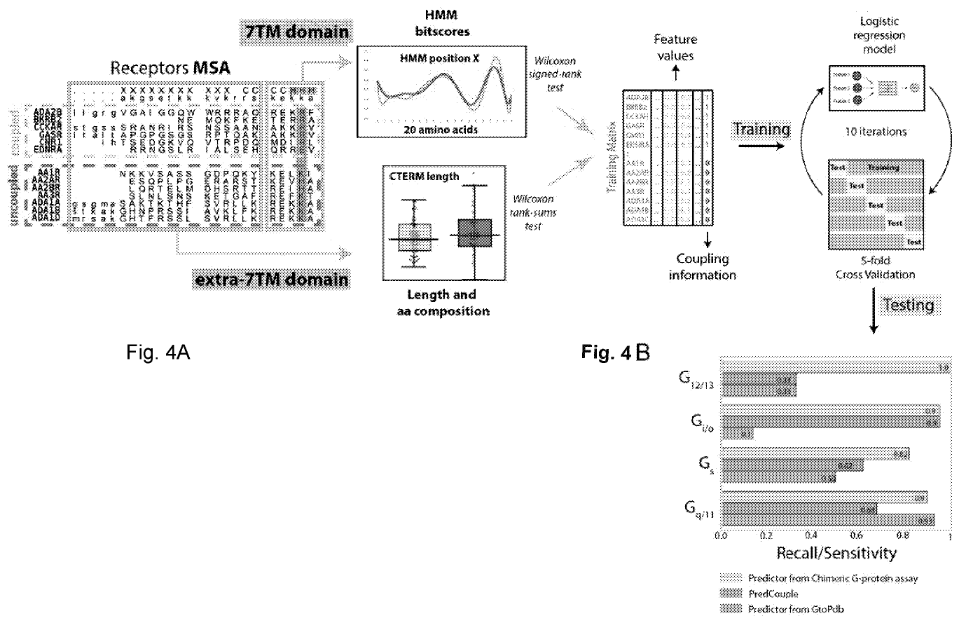

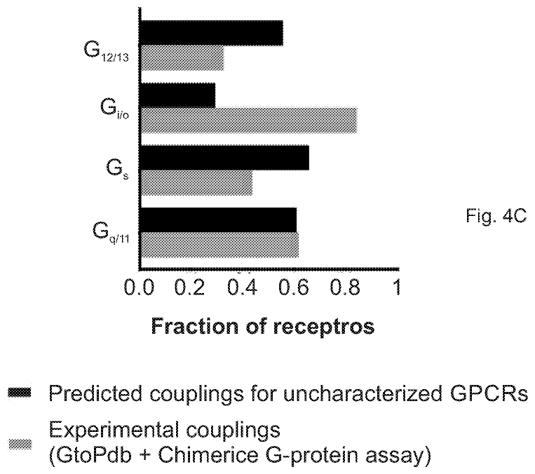

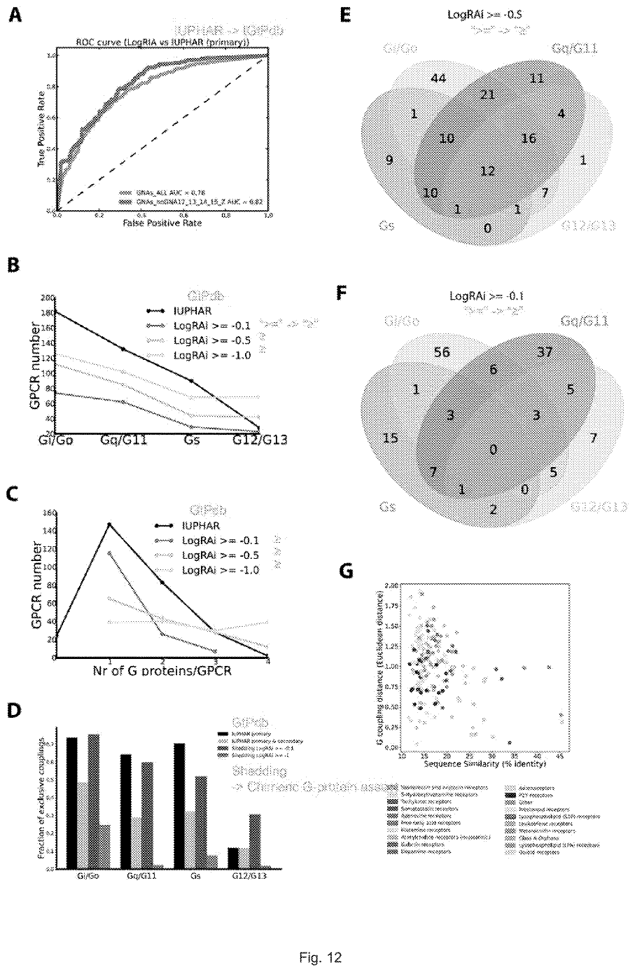

The inventors compared coupling data from the inventive chimeric G-protein-based assay with that of GtoPdb. For each of the four G-protein subfamilies, they defined positive coupling if any member of the subfamily scored LogRAi ≥−1 and negative coupling if all of the members scored LogRAi <−1 (FIGS. 3A-B). ROC analysis gives AUC=0.78 (FIG. 12A) when considering high-confidence known coupling data and suggested a threshold of LogRAi ≥−1.0 (optimizing TPR while minimizing FPR; see section EXPERIMENTAL MODEL AND SUBJECT DETAILS below) for defining true couplings. The assay also showed other broad similarities to GtoPdb, including G_{i/o} being the most common, and G_{12/13} the least (FIGS. 3C-D). They also recapitulated that the majority of receptors coupled to only one G-protein, which does not change greatly with altered LogRAi thresholds (FIG. 12C), though as expected there is greater coupling promiscuity at lower values. In addition, both GtoPdb and the inventors data (at various LogRAi stringencies) suggests G_{i/o} subunits to be the most specific, always displaying the highest fraction of exclusively bound receptors, with G_{12/13} being the most promiscuous (FIGS. 3C, 12B, D). A total of 39 promiscuous receptors are reported to couple to members of all four G-protein families (FIG. 4C), however promiscuity decreases as a function of the LogRAi threshold (FIGS. 12E, F). Overall, the dataset shows an excellent agreement with known couplings (FIG. 3D), with more than 88% of reported couplings reproduced for three classes (i.e. G_{i/o}, G_{q/11} and G_{12/13}).

The inventors found no correlation between sequence and coupling similarities, either performing pairwise comparisons on the whole set or intra-family (FIGS. 2 and 12G). Moreover, both extremes are evident: receptor pairs with low sequence similarity can have similar couplings and close homologs from the same family can show large differences (see prostanoid receptors; FIGS. 3A, B). The inventors' exploration of 11 distinct G-proteins also reveals key differences among G-protein sub-families in terms of their coupling preferences, which essentially reflects sequence similarity of the last 6 C-terminal amino acids (FIGS. 2 and 10C). For instance, several receptor families show overall coupling preferences for specific classes, like Opioid and Dopamine receptors for G_{i/o}, or Prostanoid and Adrenoceptors for Gₛ; in contrast others show more coupling promiscuity, like Endothelin, Ghrelin and Proteinase-activated receptors (FIG. 2). The great utility of the inventors' dataset is immediately clear. There are entire groups of poorly annotated (in GtoPdb) receptors that are well represented in the inventors' dataset, including ten GPCRs protease-activated receptors and P2Y receptors (P2RY10 and P2RY12), where the latter is a major target of antiplatelet agents, with roles in platelet aggregation (Dorsam and Kunapuli, 2004) and bleeding disorders (Patel et al., 2014). While P2RY10 displays specificity for both G_{i/o} and G_{12/13} subfamily members, the inventors find P2RY12 to be specific for G_{i/o}. Elsewhere, GPR132, recently emerged as a mediator of breast carcinoma metastasis (Chen et al., 2017), shows a previously unreported coupling promiscuity.

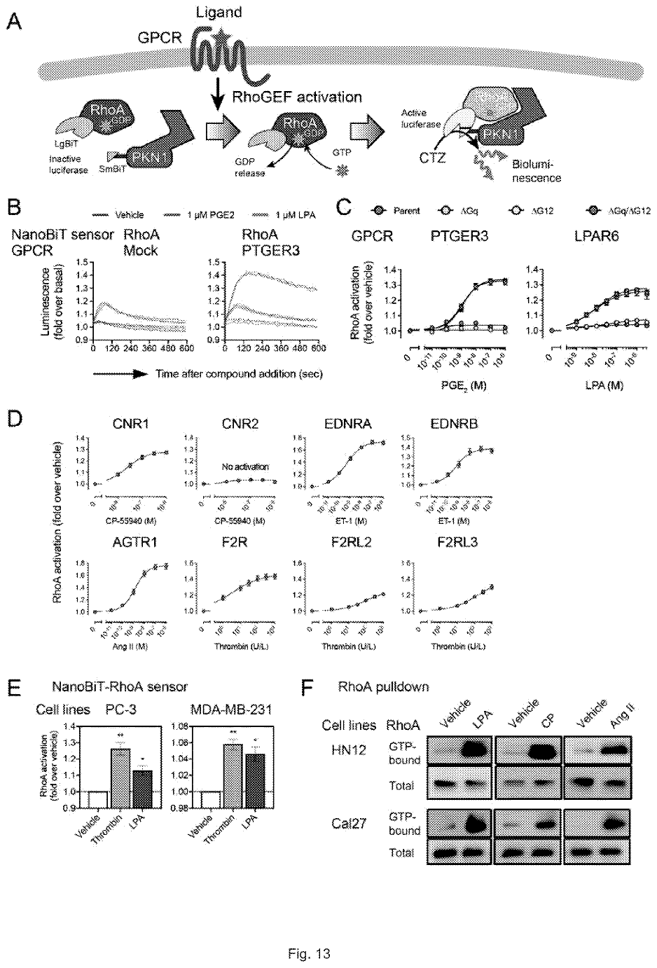

In general, more than half of the couplings detected (160/292, 55%) have not previously been reported (FIG. 3D). As expected, the biggest proportion of new couplings are G_{12/13} where the inventors' data makes up 57% of all known couplings of this type (15 out of 26 reported G_{12/G13} couplings in GtoPdb, which also considers non-Class A GPCRs). To validate that newly identified G_{12/13} couplings indeed reflect capability of endogenous G_{12/13} activation, and not artifacts of chimeric Gα subunit overexpression, they assessed TGFα shedding responses in ΔG_{q} cells (FIGS. 1A, B). The inventors tested eight GPCRs (AGTR1, CNR1, EDNRA, F2RL2, PTGER1, PTGFR, TACR1 and TBXA2R), in which G_{12/13} coupling was not registered in GtoPdb, but was detected by the chimeric G-protein-based assay. The inventors found that all of them induced TGFα shedding responses in ΔG_{q} cells and that the signals were completely silenced in ΔG_{q}/ΔG₁₂ cells (FIG. 3E). To assess a more proximal signaling event to G_{12/13} activation, they generated an inventive NanoBiT-RhoA sensor (FIGS. 13A-C; see section EXPERIMENTAL MODEL AND SUBJECT DETAILS) and found that all of tested GPCRs that were newly identified as G_{12/13}-coupled receptors (FIG. 3E), when overexpressed in HEK293 cells, induced RhoA activation (FIG. 13D). The inventors also found that thrombin also activated RhoA, presumably by activating its receptors (F2L, F2RL2 and/or F2RL3) that were endogenously expressed in PC-3 and MDA-MB-231 cells (FIG. 13E). In HN12 cells and Cal27 cells, a RhoA pulldown assay showed that CP-55940 (CP; likely via CNR1, but not CNR2; FIG. 13E) and Ang II induced activation of RhoA (FIG. 13F). Together, these data demonstrate that the G_{12/13}-coupled receptors identified by the chimeric G-protein-based assay induce RhoA activation in overexpressed HEK293 cells and/or endogenously expressed cell lines.

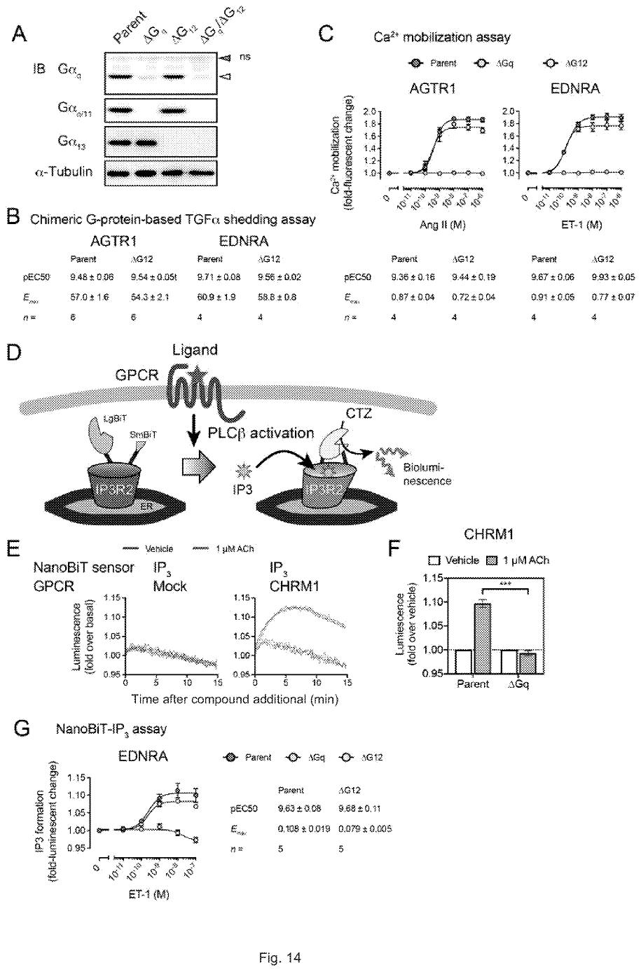

To test whether apparent unchanged TGFα shedding responses in ΔG₁₂ cells as compared with those in the parental cells (AGTR1 and EDNRA) arose from enhanced or compensated G_{q/11} signaling in ΔG₁₂ cells, the inventors analyzed G-protein expressions and performed a Ca²⁺ mobilization assay and a NanoBiT-IP3 assay (FIG. 14; see section EXPERIMENTAL MODEL AND SUBJECT DETAILS), both of which selectively measured G_{q/11} signaling (FIGS. 14C, F). Expression levels of Gα_{q} and Gα₁₁ were unchanged in ΔG₁₂ cells, nor was that of Gα₁₃ in ΔG_{q} cells (FIG. 14A). Both Ca²⁺ and IP₃ responses in ΔG₁₂ cells were comparable to those in the parental cells (FIG. 14C, G). Thus, TGFα shedding responses in ΔG₁₂ and ΔG_{q} cells are an accurate reflection of G_{q/11}- and G_{12/13} signaling.

### Sequence Features Indicative of Coupling Specificity

The inventors used a statistical model to identify sequence features associated with each of the eleven couplings determined above (all details given in section EXPERIMENTAL MODEL AND SUBJECT DETAILS). Briefly, the inventors used sequence alignments for each coupling group to define residues and more general compositional features (e.g. C-terminal or IC3 length, charge distributions, etc.) found to be statistically associated to coupling for each G-protein. These were used to train and test a machine learning (Logistic regression) predictor (FIG. 4A) and identify the features most predictive for each G-protein.

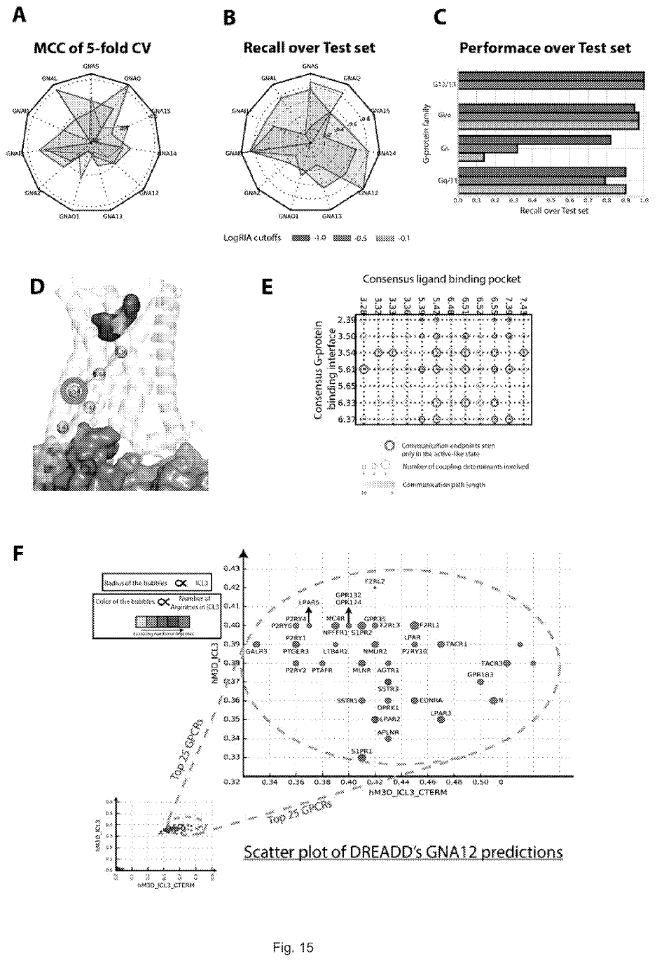

The inventive predictor performs better than another available coupling prediction approach (PredCouple) (Sgourakis et al., 2005a) in predicting known couplings not used during training for all coupling groups, but particularly for G_{12/13}, which is expected since few data were available to train such predictors previously (FIG. 4B). Note that same predictor trained only with known couplings from GtoPdb (Harding et al., 2018) performed worse (FIG. 4B) as might be expected. Using a stricter LogRAi cutoff to define coupling groups, led to a general decrease in performance during the testing phase, except for G_{i/o} (FIGS. 15A-C). The poorer performance, for the Gₛ subfamily, which also shows a poorer overlap between the chimeric G-protein-based assay and GtoPdb (FIG. 3D), is probably a consequence of the fact that the chimeric system does not capture all sequence determinants emerging for Gₛ. Nevertheless, this tool can be exploited to illuminate the transduction mechanisms of less characterized receptors. Indeed, for the 61 receptors (21% of 286 Class A GPCRs) lacking coupling information from either GtoPdb or the chimeric G-protein-based assay, the inventors predict a prevalence of Gₛ followed by G_{q/11} and G_{12/13} couplings, the latter contrasting with the smallest fraction among experimental couplings (FIG. 4C). For example, P2RY8 is readily predicted to be coupled to G_{12/13}, being consistent with a report of mutual exclusive mutations in lymphomas between the P2RY8 and the GNA13 genes, which implies a putative functional link (Muppidi et al., 2014).

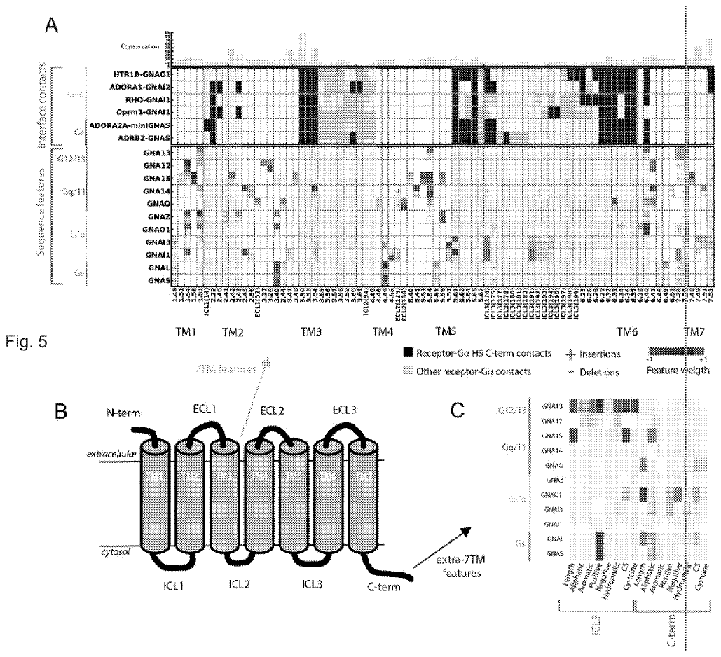

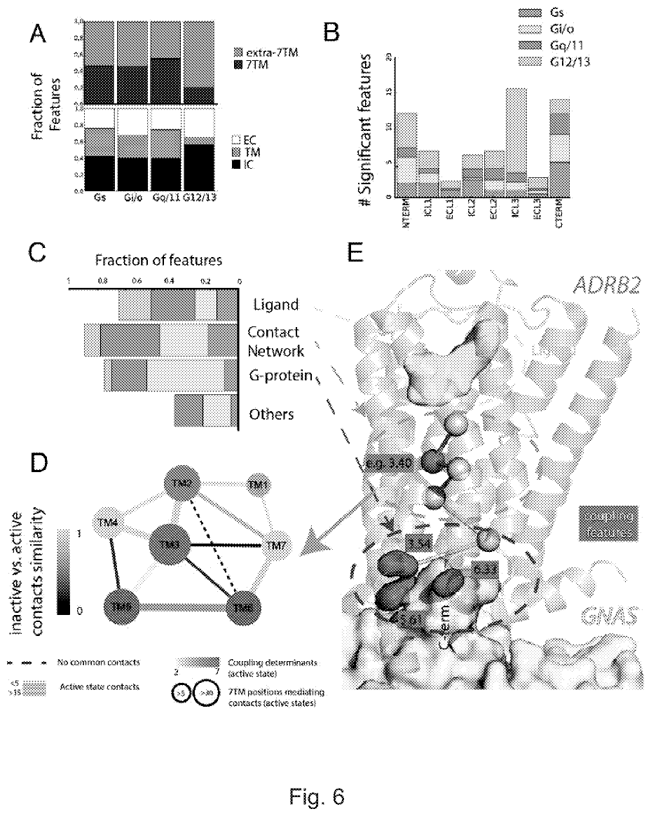

The inventive model identified different combinations of sequence features important for each coupling group (FIG. 5). After training, different weights are assigned to each feature in the logistic function to achieve optimal prediction performances, thus highlighting the most relevant determinants for each coupling (FIG. 5; see section EXPERIMENTAL MODEL AND SUBJECT DETAILS). Significant features are more abundant at the cytosolic side of the receptor (FIGS. 6A, B) including many at the known G-protein binding interface (e.g. TM3, TM5, TM6 and ICL3), but also within the core of the structure, mainly contributing to a contact network and could thus mediate specific conformational differences required to accommodate a particular G-protein (FIG. 6).

Surprisingly, only a few significant positions (12 of 51 or 23%) overlap with residues lying directly at known GPCR/G-protein interfaces (FIGS. 5A and 6C). These include ICL3, TM5 and TM6 positions associated with G_{i/o} missing from Gₛ (e.g. 5.61, ICL3: 174, 191-194) that are likely responsible for specificity. Several other positions (11, 21%) are immediately adjacent to direct contacts, suggesting they could nevertheless affect these interfaces. This is logical as some of the contacting positions are typically highly conserved across GPCRs (e.g. the DRY or NPxxY motifs). Overall, the majority (or 90%) of significant positions within the 7TM bundle mediate intra- or inter-protein contacts with either G-proteins or ligands. The majority of significant positions (29, 57% of the total) appear to mediate active-like state specific intramolecular contacts, which the inventors uncovered by comparing functional state specific contact networks (i.e. active-like and inactive-like) from three-dimensional (3D) structures (FIGS. 6C-E; see section EXPERIMENTAL MODEL AND SUBJECT DETAILS below). Helices TM3, TM5 and TM6 undergo major rewiring of their intramolecular contacts upon receptor activation and display the highest content of significant coupling features in the active-like network (FIG. 6D). This further stresses their role as master regulators of receptor activation and G-protein recognition (Koehl et al., 2018). Residues previously described as universal mediators of receptor activation participate to this network as either endpoints (6.37) or mediators (3.46 and 7.53) of the shortest paths linking the ligand and G-protein binding pockets (FIGS. 15D, E; see section EXPERIMENTAL MODEL AND SUBJECT DETAILS below).

Several other features lie within regions outside the 7TM bundle, particularly in the ICL3 or C-terminal regions (FIGS. 5C and 6A, B), that are not usually visible in experimental structures (with the exception of ICL3 in some G_{i/o} complexes), but which nevertheless play critical roles in signaling (Venkatakrishnan et al., 2014). There is broadly an equal contribution of positions from within or outside of the 7TM bundle across all families, with a greater prevalence of the outside positions for the G_{12/13} subfamily (FIGS. 6A, B).

### Data Driven Design of a G₁₂-Specific DREADD

The prominent roles for ICL3, and to a lesser extent the C-terminus, for G_{12/13}-coupled receptors, where length and electrostatic charge are predicted to be important for coupling (FIGS. 5B, C), together with the lack of structure and tools for probing G_{12/13} signaling prompted the inventors to develop a new chemogenetic receptor for studying G_{12/13} coupling. DREADDs are engineered receptors that permit spatial and temporal control of G-protein signaling in vivo, being thus of great use in studying and manipulating signaling (Urban and Roth, 2015; Wess et al., 2013). DREADDs derived from the muscarinic acetylcholine (Ach) receptors are widely used in combination with clozapine-N-oxide (CNO), a synthetic, biologically inert ligand. To date, DREADDs coupling to Gₛ, G_{i/o} and G_{q/11} (M3D-Gₛ, M4D and M3D, respectively) have been developed (Armbruster et al., 2007; Guettier et al., 2009), but there is no yet a G_{12/13}-coupled DREADD available, which the inventors sought to design using their inventive predictor.

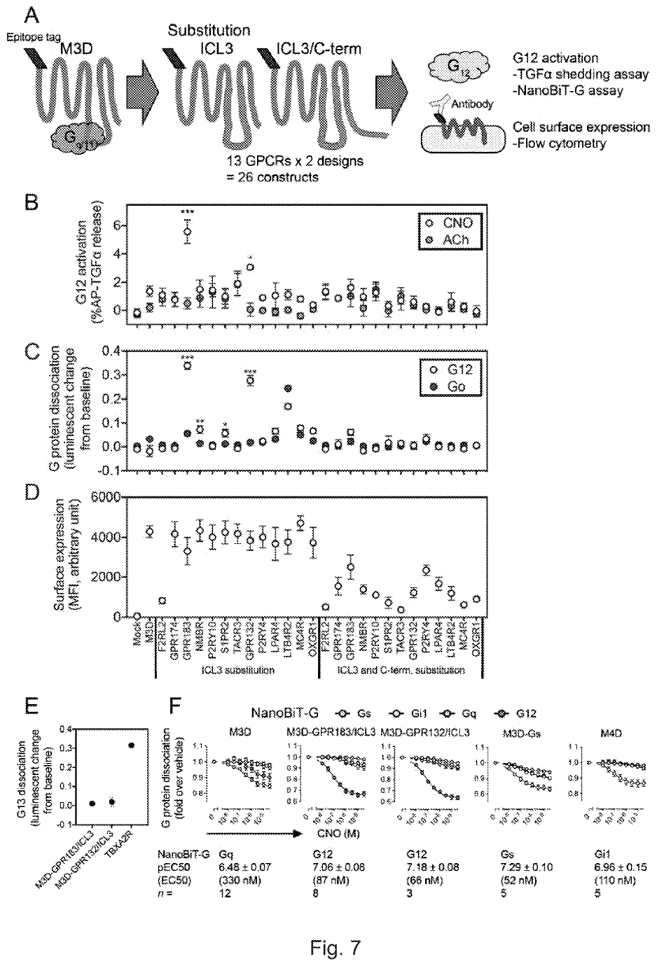

The design of M3D-Gₛ involved a strategy of substituting both ICL2 and ICL3 of the G_{q/11}-coupled M3D with those of Gₛ-coupled β1AR (Guettier et al., 2009). In the inventors' analysis, a major feature contributing to G_{12/13} coupling was ICL3, followed by the C-terminal tail (FIGS. 5C and 6B). The inventors thus explored whether these features would be sufficient to induce such signaling in M3D. The inventors first predicted the probability of G₁₂ coupling for M3D chimeras containing ICL3 swapped from all other GPCRs alone or in combination with C-terminal stretches (FIGS. 7 and 15F). Among all possible GPCR constructs (144 ICL3 swapped chimeras and 144 dual ICL3/C-terminus chimeras), the inventors selected the top 10 predictions of each chimera type (13 GPCRs in total by excluding overlaps and selecting representative constructs when multiple members from one GPCR were predicted) leading to 26 constructs (FIG. 7A). The inventors functionally screened G₁₂-coupling activity of the M3D-based chimeras using two assays. In the first, the chimera construct was expressed together with the AP-TGFα reporter (SEG ID No. 93 to 96) in ΔG_{q} HEK293 cells, in which G₁₂ signaling is selectively detectable (FIG. 7B). The inventors measured TGFα shedding response upon CNO or Ach stimulation. Among the 26 constructs screened, chimeras with the GPR183-derived ICL3 substitution (M3D-GPR183/ICL3) and the GPR132-derived ICL3 substitution (M3D-GPR132/ICL3) showed significant G₁₂ signaling (P values <0.05) (FIG. 7B). Ach did not induce detectable G₁₂ signaling in any of the tested constructs (FIG. 7B). In the second assay, the chimera construct was expressed together with NanoBiT-G₁₂ in the parental HEK293 cells and stimulated with CNO. As a negative-control counter experiment, the inventors used NanoBiT-G. since their preliminary experiment indicated a minor coupling of some chimeras to Gₒ. NanoBiT-G₁₂ screening identified four constructs (M3D-GPR183/ICL3, M3D-GPR132/ICL3, M3D-P2RY10/ICL3 and M3D-NMBR/ICL3) significantly (P<0.05) coupled to G₁₂ and not to Gₒ (FIG. 7C). One construct (M3D-LTB4R2/ICL3) induced both G₁₂ and Gₒ coupling with a higher Gₒ dissociation signal. In both assays, double swapped ICL3/C-terminus chimeras showed negligible G₁₂ signaling, which was in part attributable to lower surface expression of these constructs (FIG. 7D).

The ©nventors then evaluated selectivity of G-protein coupling for the two candidate constructs using the NanoBiT-G-protein dissociation assay with titrated CNO concentrations. As controls, the inventors compared with previously established muscarinic DREADDs (M3D, M4D and M3D-Gₛ) (Armbruster et al., 2007; Guettier et al., 2009). They tested representative NanoBiT-G-proteins (Gₛ, Gₒ, G_{q} and G₁₂) from the four subfamilies. The NanoBiT-G-protein assay correctly measured primary coupling of the three established DREADDs (M3D, M4D and M3D-Gₛ for G_{q}, Gₒ and Gₛ, respectively; FIG. 7E). M3D-GPR183/ICL3 and M3D-GPR132/ICL3 constructs showed robust G₁₂ dissociation signal while dissociations of the other G-proteins were much lower than those of G₁₂. None of the DREADD constructs induced significant NanoBiT-G₁₃ dissociation (FIG. 7E). The EC₅₀ values of CNO for each primary-coupling G-protein were in a subnanomolar range (0.1-1 μM) for all of the DREADDs (FIG. 7F). Thus, the constructs M3D-GPR183/ICL3 and M3D-GPR132/ICL3 are new G₁₂-selective DREADDs.

### Discussion

The extensive dataset provided according to the present invention greatly expands known GPCR/G-protein couplings and provides better resolution by considering all 11 specific human G-proteins rather than subfamilies. The inventive assays, resource and accompanying predictor (available at gpcr.russelllab.org) can be used for a host of biological and pharmaceutical applications. For example, the inventive TGFα shedding assay, applied to AGTR1, demonstrates the promise to develop sub-G-protein biased ligands (i.e. discriminating one G-protein signaling from another), which have recently attracted attention because of their potentials for therapeutic-signal-targeted medicine with reduced on-target side effects (Violin et al., 2014). Most importantly, the extensive dataset provides the first coupling information for many receptors (e.g. protease-activated or P2Y receptors), shows differences in G-proteins in the same family (e.g. prostanoid receptors) and, in particular, identifies dozens of receptors coupled to the previously understudied G_{12/13} (Rho signaling).

The G_{12/13} subfamily remains challenging to study owing to lack of well-established methods for assessing signaling. The inventive TGFα shedding assay combined with ΔG_{q} cells is an excellent platform for selective measurement of G_{12/13} signaling with high robustness and throughput, and in the future will enable precise characterization of receptors and their ligands. Other assays developed in this invention (the chimeric G-protein-based TGFα shedding assay, the NanoBiT-G-protein dissociation assay and the NanoBiT-RhoA sensor) will also be useful for cross-validating results. G_{12/13} signaling is also implicated in immune processes and various diseases (Herroeder et al., 2009; Suzuki et al., 2009), including receptors S1PR2 and P2RY8 in B cell lymphoma (Muppidi et al., 2014; O'Hayre et al., 2016). Agonists for G_{12/13}-coupled receptors in lymphocytes can attenuate immune responses and antagonists could potentially boost them, both of which offer attractive possibilities for future therapies. A list of the expanded members of G_{12/13}-coupled receptors identified here will provide a basis for such drug development. Indeed, some of the inventors' newly identified G_{12/13}-coupled GPCRs (e.g., CNR1, FFAR1, GHSR, GPR35, HRH2, HTR2C) are already targets for agonists approved as therapeutics (Hauser et al., 2017), suggesting additional possibilities for drug repurposing. Transgenic mice expressing the inventors' new G₁₂-coupled DREADD could help to explore G₁₂ signaling and ultimately develop such therapies.

Integrating this large GPCR/G-protein dataset with information about protein sequence and structure has identified numerous insights into how receptors selectively interact with G-proteins. Several recent structures have provided insights into the complex landscape governing GPCR coupling specificity, which is complicated by multiple factors including conformational plasticity, kinetics, ligand biasing and G-protein pre-association (Capper and Wacker, 2018). While previous efforts successfully identified sequence and structural features that determine coupling selectivity in G-proteins (i.e. the barcode), a systematic identification of receptor determinants is still lacking. The present invention identifies several features that agree with what is already known. Generally, TM3, TM5 and TM6 have the greatest number of predicted coupling features, suggesting the importance of ICL2, TM5, ICL3 and TM6 in determining complementarity to the G-protein barcode.

One potential issue with the results presented according to the present invention is the use of inventive chimeric Gα subunits, where only the 6 C-terminal amino acids are used to assess ligand-induced GPCR activation. This necessarily misses contributions of the remaining (backbone) region of the Gα subunits. However, the good agreement with known couplings (FIG. 3D) suggests that these effects are not predominating. Moreover, relative contributions (or synergistic effects) of C-terminus and backbone to coupling selectivity seem to differ among GPCRs (FIG. 4A). Ultimately, an extensive G-protein coupling dataset considering native a subunit sequences will naturally provide a more complete view of coupling determinants.

One would expect naively that coupling determinants would only lie at the interface between G-proteins and receptors and that a few simple sequence changes would account for selectivity. Decades of sequence gazing have failed to find such simple explanations. Recent receptor/G-protein complexes suggest that additional features outside the interface, such as an internal network of polar contacts, induce a greater rigidity of TM6 and lead to a preference of G_{i/o} over Gₛ. Many of the inventors' predicted sequence features away from the interface indeed participate in intra-molecular contact networks linking ligand and G-protein binding sites. The inventors speculate that these features allow allosteric and dynamic control of a G-protein binding interface of GPCRs possibly by stabilizing a specific intermediate state of a receptor/G-protein complex. The inventors also find a general tendency for TM5, ICL3 and TM6 insertions in G_{i/o}-coupled, and deletions in Gₛ-coupled receptors, which broadly agrees with the notion that the bulkier side-chains of the Gₛ G-protein C-terminus can only be accommodated by larger and more flexible crevices found in Gₛ-specific receptors (FIG. 7A).

The inventors predicted many G protein-coupling features to lie outside of the 7TM bundle. For example, ICL3 contains features for G_{12/13} coupling, the importance of which is verified by the successful generation of ICL3-swapped DREADDs. G_{12/13} is the receptor class where the inventors predict the smallest number of significant features overlapping with G-protein interface residues (FIGS. 5A and 6C) and the greatest fraction of features outside the 7TM bundle, particularly in ICL3 (FIG. 6A). Since the ICL3 is typically disordered (i.e. lacks a pre-defined structure), it is possible that the fewer specific couplings observed for G_{12/13} receptors (FIG. 3C) are a consequence of the lack of well-defined contact points in the receptor structure. Since (non G_{12/13}) G protein/GPCR complex structures show limited, but nevertheless G-protein class specific, interactions between ICL3 and flanking amino acid residues (i.e. TM5/ICL3 for Gₛ and ICL3/TM6 for G_{i/o} complexes; FIG. 5A), the inventors speculate that G_{12/13} receptors might also engage in ICL3 and Helix 5 in the Gα subunit-specific interactions that are likely different from Gₛ or G_{i/o} (or that an ICL3-Helix 5 interaction occurs during an intermediate state).

The present invention has demonstrated the power of integrating a new, powerful assay with systematic data analysis to provide new insights in molecular mechanism. With the extensive analysis, the inventors devised both biological and computational tools that will advance understanding of how cells respond to extracellular signals. Integrating the inventive resources with other datasets, such as genomic sequencing, transcriptomics, proteomics, metabolomics, and/or by considering other members of GPCRs mediated pathways, will provide new means to quantify downstream signaling in normal and pathological conditions, and provide considerable possibilities for new therapies and personalized medicine.

### DETAILED DESCRIPTION OF THE FIGURES

FIG. 1 relates to a Chimeric G-protein-based TGFα shedding assay to probe interaction between an active GPCR and a C-terminal tail of a Gα subunit.

FIG. 1(A) discloses a schematic description of the mechanism of the TGFα shedding assay. G_{q/11}- and/or G_{12/13}-coupled receptors induce activation of a membrane-bound metalloprotease ADAM17, which is endogenously expressed in HEK293 cells, and subsequent ectodomain shedding of the alkaline phosphatase-fused TGFα (AP-TGFα) construct. AP-TGFα release into conditioned media is quantified through a colorimetric reaction. Parental HEK293 cells and cells devoid of the Gα_{q/11} subunits (ΔG_{q}), the Gα_{12/13} subunits (ΔG₁₂) or the Gag/11/12/13 subunits (ΔG_{q}/ΔG₁₂) were used in the TGFα shedding assay.

FIG. 1 (B) discloses graphs of blunted TGFα shedding response in the HEK293 cells devoid of the G_{q/11} and the G_{12/13} subfamilies. GPCRs known to couple with G_{12/13} (LPAR6 and PTGER3), G_{q/11} (CHRM1 and HRH1) and both (GALR2 and GHSR) were examined for ligand-induced TGFα shedding responses in the parental HEK293 cells or the indicated G-protein-deficient cells. Symbols and error bars represent mean and SEM, respectively, of 3-6 independent experiments with each performed in triplicate.

FIG. 1 ©) discloses a schematic description of the chimeric G-protein-based TGFα shedding assay in ΔG_{q}/ΔG₁₂ cells. A test GPCR is expressed together with one of 11 chimeric Gα subunits harboring C-terminal 6-amino acid substitution in ΔG_{q}/ΔG₁₂ cells and restoration of ligand-induced AP-TGFα release response is measured. Note that there are 11 unique C-terminal sequences for the 16 human Gα subunits (the C-terminal 6-amino acid sequences of Gαᵢ₁, Gαᵢ₂, Gαₜ₁, Gαₜ₂ and Gαₜ₃ and those of Gα_{q} and Gα₁₁ are identical; also see FIGS. 10A-C) and that the invariant leucine is encoded at the −7 position. The C-terminally truncated Gα_{q} construct (Gα_{q} (ΔC)) is used for a negative control.

FIG. 1 (D) discloses representative data for the chimeric G-protein-based assay. TBXA2R was expressed with one of the 11 Gα_{q}, constructs or the Gα_{q} (ΔC) and treated with titrated concentration of a ligand (U-46619). AP-TGFα release responses were fitted to a sigmoidal concentration-response curve (upper panels). G-protein coupling is scored as logarithmic values of relative intrinsic activity (RAi), which is defined as an Eₘₐₓ/EC₅₀ value normalized by the highest value. Symbol size is proportional to Eₘₐₓ, which reflects fitting quality. During data processing, a concentration-response curve that failed to converge or has an Eₘₐₓ value of less than 3% AP-TGFα release, or a RAi value of less than 0.01 were defined as LogRAi value of −2. Data for the concentration-response curves are from a representative experiment (mean±SD of triplicate measurements). Each LogRAi plot denotes single experiment and bars and error bars are mean±SEM (n=4).

FIG. 2 relates to signatures of G-protein coupling determined by the chimeric G-protein-based assay and discloses a heatmap of the LogRAi values for the 148 receptors of the chimeric G-protein-based assay. Cell colors range from blue (LogRAi=−2) to red (LogRAi=0). Receptors (columns) and G-proteins (rows) are rearranged according to the dendrogram of the full linkage clustering of the distance matrix calculated from the coupling profiles. Receptor gene symbols are colored according to family membership as reported in GtoPdb. Heatmap was generated through the scipy library (https://www.scipy.org/).

FIG. 3 relates to a comparison between dataset of the chimeric G-protein-based assay and GtoPdb and validation of G_{12/13} signaling for the newly characterized GPCRs.

FIG. 3 (A) discloses a schematic classification of the LogRAi scores and its comparison with GtoPdb. An example heatmap of LogRAi scores for the eight prostanoid receptors is shown, with a LogRAi cutoff of −1 to binary-classify the data into coupled (Y) or uncoupled classes. G-protein coupling from GtoPdb (subfamily levels) is overlaid.

FIG. 3 (B) discloses a schematic combined binary coupling/non-coupling data for each of the four G-protein subfamilies.

FIG. 3 (C) discloses Venn diagrams with the numbers of receptors coupled to each G-protein subfamily in the chimeric G-protein-based assay (LogRAi ≥−1).

FIG. 3 (D) discloses Venn diagrams of receptor couplings to the four G-protein families according to the chimeric G-protein-based assay (LogRAi ≥−1) and GtoPdb.

FIG. 3 (E) discloses GPCRs that were identified as being coupled with G_{12/13} by the chimeric G-protein-based assay were examined for their ability to engage and activate native, endogenous G_{12/13} in HEK293 cells. As indicated GPCR was expressed in the parental, ΔG_{q}, ΔG₁₂ and ΔG_{q}/ΔG₁₂ cells with the AP-TGFα reporter construct, but not with a chimeric Gα subunit, and its ligand-induced response was assessed. Note that in all of the tested GPCRs, AP-TGFα release response occurred in ΔG_{q} cells, but was completely silenced in ΔG_{q}/ΔG₁₂ cells, showing induction of G_{12/13}-dependent signaling. Symbols and error bars represent mean and SEM, respectively, of 3-6 independent experiments with each performed in triplicate.

FIG. 4 relates to the development of G-protein coupling predictor.

FIG. 4 (A) discloses a schematic workflow of the procedure: features are extracted from sub-alignments of coupled and uncoupled receptors to a particular G-protein; features are used to generate a training matrix which is employed to train a logistic regression model through a 5-fold cross validation procedure.

FIG. 4 (B) discloses a schematic overview on the final model tested on reported couplings not previously seen during training and compared to PredCouple.

FIG. 4 (C) discloses highly confident predicted couplings (coupling probability >0.9) for 61 Class A GPCRs lacking information about transduction from both GtoPdb or the chimeric G-protein-based TGFα shedding assay (black) vs. receptors with experimental coupling information (gray).

FIG. 5 relates to featured residues in GPCRs involved in G-protein coupling selectivity.

FIG. 5 (A) discloses a schematic overview on a comparison of significant coupling features weights for the 11 G-proteins (bottom), interface contacts of 6 available GPCR-G-protein complexes (central) and 7TM domain position conservation (top). On the bottom panel are all the features (columns) that are found to be statistically significant (P<0.05) for at least one coupling group (rows). Each cell is colored based on coefficient of the given feature in the decision function of the corresponding coupling group (i.e. weight), with negative and positive values colored red and green respectively. Coupling features at 7TM domain with significantly different amino acid distributions are characterized by two values, representing the weights of the bitscores obtained from the coupled (top sub-cell) and not coupled (bottom sub-cell) HMMs for each G-protein. Insertions (i.e. positions present only in the coupled subset) or deletions (i.e. positions present only in the uncoupled subset) are indicated with a gray “+” and “−”. Black/grey boxes in the center show contacts mediated by the last 6 a.a. of α5 C-term (black) and contacts mediated by other positions of Gα subunit (grey). Top bars shows conservation profiles for PFAM 7tm_1 positions obtained by calculating the information content from HMM positions bit scores (Wheeler et al., 2014).

FIG. 5 (B) discloses a schematic overview of the 7TM topology indicating the regions contributing to the features.

FIG. 5 (C) discloses a schematic overview on significant coupling feature weights for the 11 G-proteins (same color codes as in A) of extra-7TM features of ICL3 and C-term, including length and amino-acid composition.

FIG. 6 relates to functional analysis of residues linked to coupling selectivity.

FIG. 6 (A) discloses in the upper panel a distribution of coupling feature fractions for intra- and extra-7TM portions. The formers comprise the 7TM helical bundle only, while the latters the N- and C-terminals, ECLs and ICLs; FIG. 6(A) discloses in the lower panel a distribution of the coupling feature fractions within transmembrane sectors (i.e. extracellular-EC, transmembrane-TM, intracellu-ar-IC). Extra- and intra-cellular portions are defined by ECL and ICL regions plus 5 helical positions preceding and following them.

FIG. 6 (B) diascloses a distribution of the fractions of coupling significant features outside of the 7TM bundle.

FIG. 6 (C) discloses a distribution of coupling feature fractions (relative to the total number of positions of the same class) within functional sites (i.e. mediating either ligand/G-protein binding or intra-molecular contacts).

FIG. 6 (D) discloses a graph representing intra-molecular contacts within 7TM helices. Each helix is represented by a node, whose diameter is proportional to the number of helix positions mediating contacts in the contact network derived from active-like structures and whose color (red scale) is proportional to the number of significant coupling features present in the corresponding region. Edges represent contacts between 7TM helices, where width is proportional to the number of contacts in the active-like contact network, while color scale (gray) is proportional to the similarity degree (calculated as a Jaccard index) between contacts mediated in the active- and inactive-like contact networks.

FIG. 6 (E) discloses a three dimensional schematic view of the ADRB2-GNAS complex (PDB: 3SN6) (Rasmussen et al., 2011) with side chains of coupling features at G-protein-binding sites depicted as red surfaces. A representative coupling feature at intra-molecular contacts sites (i.e. position 3.40) is depicted as a red sphere mediating one of the shortest paths linking the ligand and G-protein binding pockets (wheat sticks and spheres). The ligand and GNAS (Gαₛ) are depicted as cyan and pale-yellow surfaces, respectively.

FIG. 7 relates to generation of G₁₂-coupled designer GPCRs.

FIG. 7 (A) discloses a schematic view of generating and assessing ICL3- or ICL3/C-terminus-swapped constructs from G_{q/11}-coupled M3D. Based on the predictor scoring (FIG. 15F), the inventors selected 13 GPCRs and made 26 constructs. FIGS. 7 (B-D) discloses screening graphs of M3D-derived chimeric constructs. G₁₂ signaling of the constructs assessed by the TGFα shedding assay in the ΔG_{q} cells treated with 10 μM clozapine N-oxide (CNO) or 10 μM acetylcholine (ACh) (B). Activation of G₁₂ and Go was measured by the NanoBiT-G-protein dissociation assay with 10 μM CNO (C). Gα₁₂-Lg or Gαₒ-Lg was co-expressed with Sm-Gyt1. Changes in decreased luminescent signals are inversely plotted in the y-axis. (C). Surface expression of the M3D-derived chimeric constructs was assessed by a flow cytometry using an anti-FLAG epitope-antibody, followed by a fluorescently labeled secondary antibody (D). Symbols and error bars represent mean and SEM, respectively, of 4-8 independent experiments with each performed in duplicate or triplicate. *, P<0.05; **, P<0.01; ***, P<0.001 (two-way ANOVA, followed by Sidak's multiple comparison tests).

FIG. 7 (E) disclose graphs on lack of G₁₃ activation by the new DREADD constructs. Dissociation signals of the NanoBiT-G₁₃ protein were assessed by using 10 μM CNO (M3D-GPR183/ICL3 and M3D-GPR132/ICL3) and 1 μM U-46619 (TBXA2R). Symbols and error bars represent mean and SEM, respectively, of 3-11 independent experiments with each performed in duplicate.

FIG. 7 (F) disclose graphs on concentration-response curves for G-protein activation by DREADD constructs. Previously established DREADDs (G_{q/11}-coupled M3D, G_{i/o}-coupled M4D and Gₛ-coupled M3D-Gₛ) and the newly generated DREADDs (M3D-GPR183/ICL3 and M3D-GPR132/ICL3) were profiled for their G-protein coupling using representative members (Gₛ, Gᵢ₁, G_{q} and G₁₃) of the 4 G-protein subfamilies. Symbols and error bars represent mean and SEM, respectively, of 3-12 independent experiments with each performed in duplicate. For each DREADD, parameters for the most efficaciously coupled G-protein are shown in bottom of the panel.

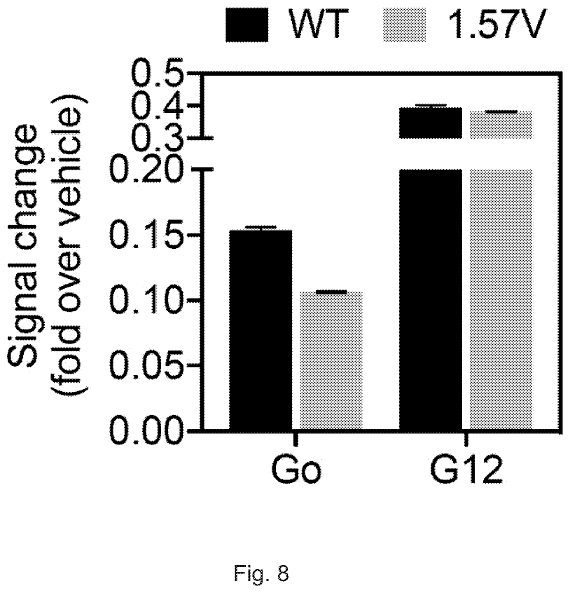

FIG. 8 relates to point mutation to enhance G-protein-coupling selectivity. G-protein activation by M3D-GPR183/ICL3 (WT) and a single amino-acid substitution at the position 1.57 with valine (1.57V) was measured by the NanoBiT-G-protein dissociation assay. Gα₁₂-Lg or Gαₒ-Lg was co-expressed with Sm-Gγₜ₁. Changes in decreased luminescent signals are inversely plotted in the y-axis. Note that in the 1.57V construct G. activation was decreased while G₁₂ activation was unchaged. The inventors found that the ICL3 substitution and/or the 1.57V mutation (position 103 in the FLAG-tagged DREADD; 93 in the original human M3 receptor) of the inventive DREADD constructs specifically binding G₁₂ subunit significantly increase selectivity.

Furthermore, for the inventive DREADD constructs, point mutations may additionally be present at Y(3.33) C and A(5.46)G, which refer to amino acid positions 149 (Y) and 239 (A), respectively, in the original human M3 receptor (Gene symbol CHRM3, disclosed in PNAS 2007, Pubmed ID 17360345). When referring to position numbers based on inventive DREADD constructs (with the 10-amino acid FLAG tag at N-terminus), they will be positions 159 (Y) and 249 (A).

FIG. 9 relates to validation of the TGFα shedding assay.

FIG. 9(A) discloses graphs on siRNA-mediated knockdown of mRNA expression. HEK293 cells transfected with a siRNA construct specific to each gene (two targeting constructs per gene) were analyzed for mRNA expression by quantitative real-time PCR. The GNAQ, the GNA11, the GNA12 and the GNA13 genes encode Gα_{q}, Gα₁₁, Gα₁₂ and Gα₁₃ subunits, respectively. mRNA levels are shown as relative values to that in control siRNA-transected cells. Bars and error bars represent mean and SEM, respectively (n=3).

FIG. 9(B) discloses an overview siRNA-mediated knockdown at protein expression levels. Lysates from HEK293 cells transfected with a mixture of the indicated siRNA constructs were subjected to immunoblot analyses using antibodies specific to Gα_{q} (an open arrowhead), Gα_{q/11}, Gα₁₃ or α-tubulin. Note that owing to a lack of a sensitive, validated antibody against Gα₁₂, immunoblot for Gα₁₂ was not assessed. ns, non-specific immunoreactive band (a filled arrowhead). FIG. 9(C) discloses graphs on knockdown of G_{q/11} attenuates AP-TGFα release induced by G_{q/11}-coupled HRH1. HEK293 cells transfected with a siRNA construct (filled symbols) and an HRH1-encoding plasmid were subjected to the TGFα shedding assay. Note that the data for the control siRNA (open symbols) are identical in all of the panels. The ADAM17 gene encode a membrane protease that cleaves the AP-TGFα reporter protein (Inoue et al., 2012). Numbers to the right of each plot indicate EC₅₀, Eₘₐₓ and RAi values obtained from sigmoidal concentration-response curves. Symbols and error bars are mean and SD (three replicate wells per one point), respectively, from a representative experiment of at least two independent experiments with similar results.

FIG. 9(D) discloses graphs on knockdown of G_{12/13} attenuates AP-TGFα release induced by G_{12/13}-coupled PTGER3. Details as for (B), but using another PTGER3 and the corresponding ligand, prostaglandin E₂ (PGE₂).

FIG. 9(E) discloses graphs on parental, ΔG_{q}, ΔG₁₂ and ΔG_{q}/ΔG₁₂ HEK293 cells were transiently transfected with a plasmid encoding N-terminally FLAG epitope-tagged GPCR (HRH1, ADRB1 or DRD1) or an empty plasmid (Mock). The transfected cells were stained fluorescently labeled with anti-FLAG tag antibody, followed by a secondary antibody conjugated with a fluorophore, and subjected to flow cytometry analysis. Data are shown in histograms and numbers at bottom of each panel indicate mean and SD (four biological replicates per one condition), respectively, of fluorescently positive percentage and mean fluorescent intensity (MFI) from a representative experiment of two independent experiments with similar results. Note that due to transient transfection, there are two peaks showing a highly expressing cell pool and poorly expressing one.

FIG. 9(F) discloses Cell surface expression levels dependent on C-terminal region of a co-expressed chimeric Gα_{q} subunit. In ΔG_{q}/ΔG₁₂ HEK293 cells, chimeric Gα subunits were individually transfected with a plasmid encoding an N-terminally FLAG epitope-tagged GPCR (HRH1, ADRB1 or DRD1). The transfected cells were stained fluorescently labeled with anti-FLAG tag antibody, followed by a secondary antibody conjugated with a fluorophore, and subjected to flow cytometry analysis. Plots in the panels denote independent experiments and bars represent mean values (n=4 or 5). MFI values that significantly differ from the control (Gα_{q} (ΔC)) are denoted by asterisks: * P<0.05 (one-way ANOVA with Dunnett's post hoc test). NS denotes not significantly different from control.

FIG. 9(G) discloses graphs on concentration-response curves of AGTR1 for the endogenous ligand (Angiotensin II, AngII) and a biased agonist ([Sar¹-IIe⁴-IIe⁸] AngII, SII). G-protein signaling activity was assessed by the chimeric-G-protein-based assay. Symbols and error bars are mean and SEM, respectively, of eight independent experiments with each performed triplicate.

FIG. 9(H) discloses ligand bias plots. For each chimeric-G-protein coupling, LogRAi values were plotted. If SII behaves as a balanced ligand, plots would be linearly aligned. Dotted lines (slope=1) were drawn crossing C-terminal Gα_{q} or Gα₁₂ chimera, indicating that SII is more biased towards G₁₂ than G_{q}. Note that activation of Gαₛ by SII was minimum and thus not included in the plot. Symbols and error bars are mean and SEM, respectively, of eight independent experiments. FIG. 9(I) discloses graphs on validation of SII bias toward G₁₂ by the NanoBiT-G-protein dissociation assay. NanoBiT-G-proteins (G_{q} and G₁₂) were expressed with AGTR1 and ligand-induced G-protein-dissociation signal was measured. Symbols and error bars are mean and SEM, respectively, of six independent experiments.

FIG. 10 relates to chimeric Gα subunits and their activity for TGFα shedding and CAMP responses.

FIG. 10 (A) discloses overview on seven C-terminal sequences (CGN numbering (Flock et al., 2015) of G.H5.20-G.H5.26) of Gα subunits among the 16 human Gα subunits. Asterisks indicate identical amino acids to one above. Note that there are 11 distinct sequences and that the −7 position (G.H5.20) is a completely conserved leucine.

FIG. 10 (B) discloses an overview on evolutionally conservation the seven C-terminal sequences of representative Gα subunits from the four G-protein subfamilies.

FIG. 10 (C) discloses an overview on chimeric G-proteins used in this study. The inventors used human Gα_{q}-based chimera with a substitution of six C-terminal amino acids. In the negative-control Gα_{q}, the seven C-terminal amino acids are truncated.

FIG. 10 (D) discloses graphs on capacity of Gα subunits to induce TGFα shedding response. Scheme of the experiment is shown in left. G_{i/o}-coupled DRD2 was co-expressed with an indicated chimeric Gα subunit or a native, full-length Gα subunit in ΔG_{q}/ΔG₁₂ cells and subjected to the TGFα shedding assay by using dopamine. Note that the Gα_{q/i1} chimera induced the most potent response and the other negative control conditions (Gαᵢ₁, Gα_{q} or an empty vector transfection (Mock)) did not induce the signal. Symbols and error bars are mean and SD (three wells per one point), respectively, from a representative experiment of at least two independent experiments with similar results.

FIG. 10 (E) discloses graphs on capacity of Gα subunits to induce TGFα shedding response. Scheme of the experiment is shown in left. The experimental design is the same as DRD2, except for usage of Gₛ-coupled PTGER2, C-terminal Gαₛ chimeras and prostaglandin E₂ (PGE₂). Note that the Gα_{q/s} chimera induced the most potent response and the other negative control conditions (Gαₛ long isoform (Gα_{sL}), Gαₛ short isoform (Gαₛs), Gα_{q} or an empty vector transfection (Mock)) did not induce the signal. Symbols and error bars are mean and SD (three replicate wells per one point), respectively, from a representative experiment of at least two independent experiments with similar results.

FIG. 10 (F) discloses overview on protein expression levels of chimeric Gα_{q} subunits. Lysates from ΔG_{q}/ΔG₁₂ cells transfected with a plasmid encoding an indicated chimeric Gα_{q} subunit were subjected to immunoblot analyses using antibodies specific to Gα_{q}, Gα_{q/11} or α-tubulin. Note that expression levels of the chimeric Gα_{q} subunits were almost equal except for the C-terminally truncated Gα_{q} (ΔC), which was previously shown to undergo spontaneous activation (Denker et al., 1992), and thereby likely to be unstable in cells owing to its tendency to separate from Gβγ subunits.

FIG. 10 (G) discloses graphs on kinetics of cAMP level upon Gₛ-coupled receptor stimulation. HEK293 cells devoid of the Gₛ subfamily (ΔGₛ, lacking Gαₛ and Gα_{olf}) (Stallaert et al., 2017) transiently expressing a cAMP biosensor (Glo-22F), a Gₛ-coupled receptor (AVPR2 or mock transfection) and a Gαₛ construct (native Gαₛ, Gαₛ-Lg or mock transfection) were loaded with D-luciferin. The cells were stimulated with an increasing concentration of arginine-vasopressin, an AVPR2 ligand, or Forskolin (FSK, 10 μM), an adenylyl cyclase activator, and luminescent signals were measured for 20 min. luminescent signals were normalized to initial counts and relative values are plotted. Each line indicates a kinetics from a single well and data are from a representative experiment of at least three independent experiments with similar results. Note that owing to preference of Forskolin to a Gαₛ-bound adenylyl cyclase (Insel and Ostrom, 2003), Forskolin-induced CAMP response was attenuated in the ΔGₛ cells. Also note that in native Gαₛ-expressing cells, owing to higher initial luminescent counts reflecting constitutive Gₛ activity, amplitude of fold change is smaller than the other conditions.

FIG. 10 (H) discloses graphs on concentration-response curves. Fold-change luminescent signals at 10 min after ligand addition in A were normalized to Forskolin response and fitted to a sigmoidal curve. Symbols and error bars are mean and SEM, respectively (n=3 or 4). Pharmacological parameters are shown at the bottom (mean±SEM). Mean pEC₅₀ values were anti-logarithmically transformed and expressed as pM values in parenthesis.

FIG. 10 (I) discloses overview on chimeric G-protein-based CAMP assay in ΔGₛ cells. A test GPCR is expressed together with one of 11 chimeric Gαₛ subunits harboring C-terminal 6-amino acid substitution in ΔGₛ cells and restoration of ligand-induced cAMP response is measured by a luminescent cAMP biosensor. The C-terminally truncated Gαₛ construct (Gαₛ (ΔC)) is used for a negative control.

FIG. 10 (J) discloses graphs on representative data for the chimeric G-protein-based CAMP assay. TBXA2R was expressed with one of the 11 Gαₛ constructs or the Gαₛ (ΔC) and treated with titrated concentration of a ligand (U-46619). Ligand-induced cAMP responses normalized to forskolin (10 μM)-induced response were fitted to a sigmoidal concentration-response curve (upper panels). G-protein coupling is scored as logarithm of RAi values. Symbol size is proportional to Eₘₐₓ, which reflects fitting quality. Data for the concentration-response curves are from a representative experiment (mean±SD of triplicate measurements). Each LogRAi plot denotes single experiment (n=5).

FIG. 10 (K) discloses graphs on comparison of the chimeric G-protein backbones in coupling profiles. Log RAi values obtained from the chimeric G_{q}-based TGFα shedding assay are plotted against the chimeric Gₛ-based CAMP assay for seven prostanoid receptors. Considered were only mean values for the plots. Note that PTGFR showed poor responses in the CAMP assay and thus not used for the comparison. Linear regression analysis was performed and 90% confidence bands of the best-fit line were shown. Mean±SD of r² values from the seven prostanoid receptors is shown at the bottom.

FIG. 11 relates to the development and validation of the NanoBiT-G-protein dissociation assay.

FIG. 11 (A) discloses schematic view of the NanoBiT-G-protein assay. Components of the NanoBiT-G-protein (typically, LgBiT-inserted Gα subunit, SmBiT-fused Gβ₁ subunit and native Gγ₂ subunit) and a test GPCR are transiently expressed in HEK293 cells. By loading with coelenterazine (CTZ), a substrate for the NanoBiT luciferase, the NanoBiT-G-protein emits bioluminescence. Stimulation with a GPCR ligand triggers exchange of a guanine nucleotide (GDP release and GTP incorporation) of the NanoBiT-G-protein and induces dissociation of Gα-Lg from Sm-Gβγ, thereby reducing bioluminescence signals. Note that both Gα and Gβγ subunits are lipidated (not shown) and localized to membrane.

FIG. 11 (B) Graph on luminescent kinetics of NanoBiT-G-proteins after GPCR ligand stimulation. Representative members (Gₛ, Gᵢ₃, G_{q} and G₁₃) from the four G-protein subfamilies were co-expressed with a corresponding coupling GPCR and stimulated with its ligand (10 μM isoproterenol, 10 μM dopamine or 1 μM U-46119) or vehicle. After ligand addition, a microplate was measured at 10-sec interval for 10 min. Each line indicates a luminescent trace of one well, normalized to an initial count, from a representative experiment of at least three independent experiments with similar results. Note that due to a time lag between manual ligand addition and beginning of measurement, NanoBiT-G₁₃ shows already dissociated signal from the initial reading.

FIGS. 11 (C-E) discloses graphs on the effect of preincubation time with CTZ. Cells expressing the indicated combination of the NanoBiT-G-protein and the test GPCR were loaded with CTZ for 15-120 min before measurements (baseline and ligand stimulation). Luminescent signals after 3-5 min ligand addition (10 μM isoproterenol, 10 μM dopamine or 1 μM U-46119) or vehicle treatment were normalized to the baseline signals (FIG. 11C). Bars and error bars represent mean and SEM of indicated numbers of independent experiments in FIG. 11E.

Changes in the luminescent signals across titrated ligand concentrations were further normalized to that of vehicle treatment and expressed as concentration-response curves (FIG. 11D). Symbols and error bars represent mean and SEM of indicated numbers of independent experiments in FIG. 11E. Parameters (signal changes and pEC₅₀ values) were calculated from the sigmoidal curves (FIG. 11E). Data are expressed as mean±SEM of indicated numbers of independent experiments. Mean pEC₅₀ values were anti-logarithmically transformed and expressed as nM values.

FIG. 11 (F) discloses graphs on validation of the NanoBiT-G-proteins by using prostanoid receptors. Seven NanoBiT-G-protein (Gₛ, Gᵢ₂, Gᵢ₃, Gₒ, G_{q}, G₁₂ and G₁₃) were profiled for the eight prostanoid receptors with their ligands shown in parentheses. Each dot represent data from independent experiments and lines and error bars indicate mean and SEM (n=5−9).

FIG. 11 (G) discloses graphs on comparison of coupling profiling between the chimeric G-protein-based TGFα shedding assay and NanoBiT-G-protein assay. Log RAi values obtained from the chimeric G-protein-based assay are plotted against G-protein dissociation signals from the NanoBiT-G-protein assay for the eight prostanoid receptors. Considered were only mean values for the plots. The Gᵢ family contains data for three members (Gα_{q/i1}, Gα_{q/i3} and Gago chimeras; NanoBiT-Gᵢ₂, NanoBiT-Gᵢ₃ and NanoBiT-Gₒ, respectively) and the G₁₂ family includes data for two members (Gα_{q/12} and Gα_{q/13} chimeras; NanoBiT-G₁₂ and NanoBiT-G₁₃, respectively). PTGER3-G₁₂ data were excluded owing to the increased luminescent signal. Linear regression analysis was performed and 95% confident intervals were shown.

FIG. 12 relates to analysis of the chimeric G-protein-based assay dataset and comparison with GtoPdb.

FIG. 12 (A) discloses a graph on Roc curve comparing the chimeric G-protein-based TGFα shedding assay couplings with GtoPdb couplings: roc curves were generated considering only GtoPdb best characterized primary couplings (i.e. reported in at least 3 publications) as binary classifier and TGFα□ shedding assay values as scores. Roc curves were calculated either considering coupling values for all the G-proteins in the chimeric G-protein-based TGFα shedding assay (gray) or by excluding poorly characterized couplings (i.e. GNA12, GNA13, GNA14, GNA15, GNAZ; red curve).

FIG. 12 (B) discloses graph on number of GPCRs coupled to G-proteins of the four families at different LogRAi thresholds in the chimeric G-protein-based TGFα shedding assay as well as in GtoPdb.

FIG. 12 (C) discloses graph on distribution of the number of reported bindings (of any of the four G-protein families) for each receptor at different LogRAi thresholds in the chimeric G-protein-based TGFα shedding assay as well as in GtoPdb.

Figures (D-F) disclose overview on fractions of specific couplings, i.e. receptors binding to members of only one G-protein family, in the chimeric G-protein-based TGFα shedding assay (dark red and orange bars for LogRAi ≥−0.1 and −1 couplings) and GtoPdb (black and grey bars for primary only and primary & secondary couplings); Venn diagrams with the numbers of receptors coupled to each G-protein family in the chimeric G-protein-based TGFα shedding assay at higher LogRAi stringencies ≥−0.5 (E) and ≥−0.1 (F).

FIG. 12 (G) discloses graph on comparison of receptor sequence and coupling profile similarities: the inventors calculated receptor pairwise sequence similarity by outputting distance matrices from ClustalO (Sievers et al., 2011). The inventors compared receptor pairwise coupling similarity by calculating the distance matrix of coupling profiles (i.e. vectors containing LogRAi values for 11 G-proteins) through the pdist function from scipy (https://www.scipy.org/).

FIG. 13 relates to validation of RhoA activation by the newly identified G_{12/13}-coupled GPCRs.

FIG. 13 (A) discloses a schematoc overview of the NanoBIT-RhoA sensor. The two fragments (LgBiT and SmBIT) of the NanoBiT luciferase are N-terminally fused to RhoA and its effector PKN1, respectively. Upon activation by exchanging GDP to GTP, GTP-bound RhoA interacts with PKN1, thereby increasing luminescent signals being measurable upon loading with CTZ.

FIG. 13 (B) discloses graphs on luminescent kinetics of the NanoBIT-RhoA sensor after GPCR ligand stimulation. HEK293 cells expressing the sensor alone (Mock) or with a test GPCR (PTGER3) were stimulated with its ligand (prostaglandin E₂, PGE₂), lysophosphatidic acid (LPA) or vehicle. After ligand addition, a microplate was measured at 10-see interval for 10 min. Each line indicates a luminescent trace of one well, normalized to an initial count, from a representative experiment of at least three independent experiments with similar results. Note that LPA stimulates LPA receptors endogenously expressed in HEK293 cells.

FIG. 13(C) discloses graphs on validation of G_{12/13}-mediated signal of the NanoBiT-RhoA sensor. PTGER3 or LPAR6 was expressed with the NanoBiT-RhoA sensor in the parental, ΔG_{q}, ΔG₁₂ and ΔG_{q}/ΔG₁₂ HEK293 cells, and ligand-induced luminescent signals were measured. Symbols and error bars represent mean and SEM, respectively, of 4 (PTGER3) and 6 (LPAR6) independent experiments with each performed in duplicate. ***, P<0.001 (t-test).

FIG. 13 (D) dioscloses graphs on NanoBiT-RhoA activation by selected GPCRs. Test GPCRs including the newly identified G_{12/13}-coupled GPCRs (FIG. 3E) were expressed together with the NanoBiT-RhoA sensor in HEK293 cells and ligand-stimulated luminescent signals were measured. Note that CP-55940-induced RhoA activation occurred in cells expressing CNR1, but not CNR2. Symbols and error bars represent mean and SEM, respectively, of 5-7 independent experiments with each performed in single measurement or duplicate. * P<0.05, ** P<0.05 as compared with vehicle treatment (one-way ANOVA with Dunnett's post hoc test).

FIG. 13 (E) discloses graphs on NanoBiT-RhoA activation through endogenously expressed GPCRs. PC-3 cells and MDA-MB-231 cells transiently expressing the NanoBiT-RhoA sensor alone were stimulated with vehicle, thrombin (1000 U L⁻¹) or LPA (1 μM), which is a potent inducer of RhoA activation in many cell types. Bars and error bars represent mean and SEM, respectively, of 3 independent experiments with each performed in triplicate. ***, P<0.001 (t-test).

FIG. 13 (F) discloses overview on RhoA pulldown assay to detect G_{12/13} activation by endogenously expressed GPCRs. HN12 cells and Cal27 cells were serum-starved and treated with 5 μM LPA, 10 μM CP-55940, 1 μM Ang II or vehicle. Cell lysates were subjected to the pulldown assay using Rhotekin-beads and precipitated GTP-bound RhoA proteins as well as input RhoA proteins (Total) were assessed by immunoblot analysis. Images of immunoblot membranes are representatives of two experiments with similar results.

FIG. 14 relates to G_{q/11} signaling in the absence of G_{12/13} for GPCRs coupled with G_{q/11} and G_{12/13}.

FIG. 14(A) discloses overview on protein expression levels of Gα subunits. Lysates from the parent, ΔG_{q}, ΔG₁₂ and ΔG_{q}/ΔG₁₂ cells were subjected to immunoblot analyses using antibodies specific to Gα_{q} (an open arrowhead), Gα_{q/11}, Gα₁₃ or α-tubulin. Note that compensatory upregulation of Gα subunits in ΔG_{q} cells (for Gα₁₃) or ΔG₁₂ (for Gα_{q} or Gα₁₁) was not observed. Also, note that owing to a lack of a sensitive, validated antibody against Gα₁₂, immunoblot for Gα₁₂ was not assessed. ns, non-specific immunoreactive band (a filled arrowhead).

FIG. 14 (B) discloses overview on parameters obtained from concentration-response curves of the chimeric G-protein-based TGFα shedding assay (FIG. 3E above).

FIG. 14 (C) discloses graphs on Ca²⁺ mobilization assay. The parent, ΔG_{q} and ΔG₁₂ cells transiently expressing AGTR1 or EDNRA were loaded with a Ca²⁺ fluorescent dye and ligand-induced Ca²⁺ mobilization was assessed. Symbols and error bars represent mean and SEM of the indicated numbers of independent experiments with each performed in duplicate. Parameters obtained from the concentration-response curves are shown at the bottom.

FIG. 14 (D) discloses a schematic overview of the NanoBiT-IP₃ sensor. The two fragments (LgBiT and SmBIT) of the NanoBiT luciferase are N-terminally and C-terminally, respectively, fused to inositol-triphosphate (IP3) receptor IP3R2. Upon activation of phospholipase CB and hydrolysis of phosphoinositides, released IP3 induces conformational change in IP3R2 and increases luciferase activity.

FIG. 14 (E) discloses a graphs on luminescent kinetics of the NanoBiT-IP₃ sensor after GPCR ligand stimulation. HEK293 cells expressing the sensor alone (Mock) or with a test GPCR (CHRM1) were stimulated with its ligand (acetylcholine, ACh) or vehicle. After ligand addition, a microplate was measured at 10-sec interval for 15 min. Each line indicates a luminescent trace of one well, normalized to an initial count, from a representative experiment of at least three independent experiments with similar results.

FIG. 14 (F) discloses a graph on validation of G_{q/11}-mediated signal of the NanoBiT-IP₃ sensor. The parental or ΔG_{q} cells transiently expressing CHRM1 and the NanoBiT-IP₃ sensor were stimulated with vehicle or ACh, and ACh-induced luminescent signal change was normalized to that of vehicle treatment. Bar and error bars represent mean and SEM, respectively, of 5 (parent) or 4 (ΔG_{q}) independent experiments with each performed in triplicate.

FIG. 14 (G) discloses graph on measurement of IP3 formation in EDNRA. The parent, ΔG_{q} and ΔG₁₂ cells transiently expressing EDNRA and the NanoBiT-IP₃ sensor were stimulated with a titrated ligand and ligand-induced luminescent signal was assessed. Symbols and error bars represent mean and SEM of the indicated numbers of independent experiments with each performed in duplicate. Parameters obtained from the concentration-response curves are shown at the bottom.

FIG. 15 relates to predictor performances, shortest path from contact network analysis, DREADD predictions scatter plot.

FIG. 15 (A) discloses a radial plot representing Matthew correlation coefficient (MCC) of 5-fold cross validation (averaged over 10 runs).

FIG. 15 (B) discloses radial plot representing Recall (Sensitivity) of the best performing predictors over the Test set.

FIG. 15 (C) discloses bar plot representing the recall (sensitivity) of the best performing predictors, trained at different LogRAi cutoffs, over the test set.

FIG. 15 (D) discloses overview on example of a shortest communication pathway, depicted on 3D cartoons of the ADRB2-GNAS complex (PDB ID: 3SN6), linking the ligand and G-protein consensus binding pocket pockets.

FIG. 15 (E) discloses a connectivity matrix displaying shortest paths (as intersecting circles) linking residues forming the ligand and G-protein consensus binding pockets (i.e. shown to form such interfaces in at least 50% of the considered structures). Circle color indicates the path length and the diameter is proportional to the number of significant coupling features found at linking positions. Circle rims are red marked if the path is exclusively found on active-like GPCR structures.

FIG. 15 (F) discloses a scatter plot of the relative coupling probabilities of chimeric sequences obtained by swapping on the hM3D backbone sequence the sequence stretches corresponding to the ICL3 alone (y axis) or in combination with the C-term (x axis) from the 148 receptors of the chimeric G-protein-based TGFα shedding assay. The zoomed caption highlights the cluster of chimeric sequences (including GPR183 and GPR132) displaying an increase of coupling probability for GNA12 compared to the reference (i.e. hM3D).

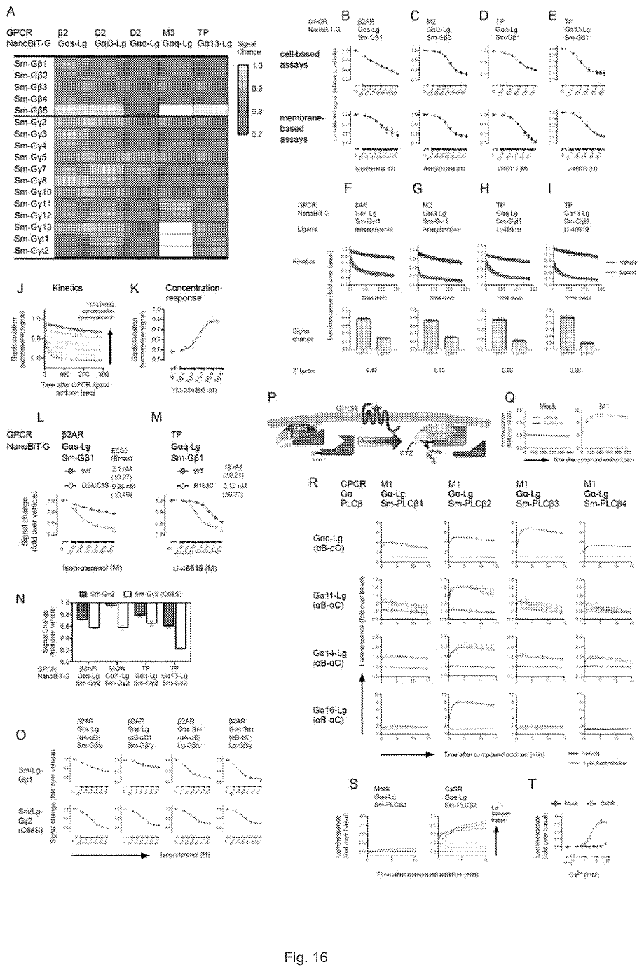

FIG. 16 relates to alidation and application of the NanoBiT-G-proteins.

FIG. 16(A) represents a heatmap comprising G-protein dissociation profiles across Gβ and Gγ subtypes. Gα-Lg along with coupling GPCR (y-axis) was co-expressed with an indicated subtype of Sm-Gβ or Sm-Gγ and stimulated with a ligand (10 μM isoproterenol for β2AR, 10 μM dopamine for D2, 10 μM acetylcholine for M3 and 1 μM U-46119 for TP). Changes in decreased luminescent signals are presented in a heatmap.

FIG. 16 (B-E) represent graphs on membrane-based NanoBiT-G-protein dissociation assay. Indicated combination of NanoBIT-G-protein and GPCR was expressed in HEK293 cells and used to cell-based assay (top panels) or membrane preparation by homogenizing cells and ultracentrifuge. The resulting membrane fraction was subjected to membrane-based assay (bottom panels).

FIG. 16 (F-I) represent graphs on robustness of the NanoBiT-G-protein dissociation assay. Indicated combination of NanoBiT-G-protein and GPCR was expressed in HEK293 cells and membrane fraction was prepared. In a 96-well plate assay, a half (48-wells) was stimulated with a ligand and the other half (48-well) was treated with vehicle. Kinetics data (top panels) and concentration responses after 3-5 min ligand addition (bottom panels) are shown. Note that in all of the four representative G-protein members, Z′ fator exceeds 0.5, indicating that the NanoBiT-G-protein dissociation assay is robust.

FIG. 16 (J-K) represent graphs on assessment of G-protein inhibitor. TP, Gα_{q}-Lg and Sm-Gγmₜ₁ were expressed in HEK293 cells and membrane fraction was prepared. After dispensed in a 96-well plate, the samples were treated with titrated concentration of YM-254890, a G_{q} inhibitor, for 30 min. The samples were then stimulated with 1 μM U-46619. Kinetics data (J) and concentration responses after 3-5 min ligand addition (K) are shown.

FIG. 16 (L-N) represent graphs on enhanced sensitivity of the NanoBiT-G-protein dissociation assay. Indicated combination of NanoBiT-G-protein and GPCR was expressed in HEK293 cells and subjected to cell-based assays. Note that lipidation-defective Gα subunit (L), constitutively active Gα subunit (M) and lipidation-defective Gγ subunits (N) show larger Eₘₐₓ (or signal change) and/or smaller EC₅₀ values, demonstrating that the NanoBiT-G-protein dissociation signal is more sensitive than the wild-type construct.

FIG. 16 (O) represent graphs on assessment of chimeric G-protein constructs. Indicated combination of NanoBiT-G-protein and GPCR was expressed in HEK293 cells and subjected to the NanoBiT-G-protein dissociation assay. Concentration responses after 3-5 min ligand addition are shown. Note that in Gα subnit, both insertion sites (αA-αB linker and αB-αC linker) as well as both fragments (LgBiT and SmBIT) were functional.

FIG. 16(P) represent a schematic view to assess NanoBiT-G-protein activation by its interaction with PLCβ (NanoBiT-G_{q}/PLC assay). Gα_{q}-Lg and Sm-PLCβ are expressed in cells together with G_{q}-coupled receptors. Ligand stimulation induces interaction between Gα_{q} and PLCβ, thereby emitting bioluminescent signals.

FIG. 16 (Q) represents graphs on luminescent kinetics of NanoBiT-G_{q}/PLC assay after GPCR ligand stimulation. Gα_{q}-Lg (LgBiT insertion at the αB-αC linker) and Sm-PLCβ₃ are expressed in cells with or without (mock) M1. The cells were treated with 1 μM acetylcholine or vehicle. Each line indicates a luminescent trace of one well, normalized to an initial count, from a representative experiment of at least three independent experiments with similar results.

FIG. 16 (R) represents graphs on combinations of the Gα_{q} family members and PLCβ subtypes. All of the 4 Gα_{q} family members (Gα_{q}, Gα₁₁, Gα₁₄ and Gα₁₆; LgBiT insertion at the αB-αC linker) and the 4 PLCβ subtypes (PLC⊕₁₋₄; SmBIT fusion at N-terminus) was tested. Note that the Sm-PLCβ2 construct shows high ligand-induced signal changes across the 4 Gα_{q} family members.

FIG. 16 (S-T) represent graphs on detection of CaSR activation by Ca²⁺. Gα_{q}-Lg (LgBiT insertion at the αB-αC linker) and Sm-PLCβ2 are expressed in cells with or without (mock) CaSR. The cells were treated with titrated concentration of Ca²⁺. Kinetics data (R) and concentration responses after 5-10 min ligand addition(S) are shown.

The present invention is explained further with the aid of the following non-limiting examples, illustrating the parameters of and compositions employed within the present invention. Unless stated otherwise, all data, in particular percentages, parts and ratios are by weight.

According to the present invention the individual features of the exemplary embodiments of the inventive aspects as disclosed in the summary, the detailed description or claims of the present application can respectively be separately combined with singular features or feature combinations of the exemplary embodiments herein below.

### Experimental Model and Subject Details

### Cells and Transfection

HEK293A cells (Female origin; Thermo Fisher Scientific) and their derivative G-protein-deficient HEK293 cells were maintained in Dulbecco's Modified Eagle Medium (DMEM 2, Nissui Pharmaceutical) supplemented with 10% fetal bovine serum (Gibco®, Thermo Fisher Scientific) and penicillin-streptomycin-glutamine (complete DMEM). Generation and characterization of the ΔGq HEK293 cells, in which null mutations were introduced into the GNAQ and the GNA11 genes by a CRISPR-Cas9 system (Schrage et al., 2015) and thus their functional products are lacking, the ΔG₁₂ HEK293 cells (lacking functional products of the GNA12 and the GNA13 genes), the ΔG_{q}/ΔG₁₂ HEK293 cells (lacking those of the GNAQ, the GNA11, GNA12 and the GNA13 genes) (Devost et al., 2017) and the ΔGₛ HEK293 cells (lacking those of the GNAS and the GNAL genes) (Stallaert et al., 2017) were described previously. The cells were regularly tested for mycoplasma contamination using a MycoAlert Mycoplasma Detection Kit (Lonza).

Transfection was performed by using a lipofection reagent, Lipofectamine® 2000 Reagent (Thermo Fisher Scientific), or polyethylenimine (PEI) solution (Polyethylenimine “Max”, Polysciences). Typically, HEK293 cells were seeded in a 6-well culture plate at cell density of 2×10⁵ cells ml⁻¹ in 2 ml of the complete DMEM and cultured for one day in a humidified 37° C. incubator with 5% CO₂. Seeding density for the ΔG₁₂ cells and the ΔG_{q}/ΔG₁₂ cells were increased to 2.5×10⁵ cells ml⁻¹ owing to slower growth of the cells than the parent HEK293 cells and the ΔG_{q} cells. For Lipofectamine® 2000 transfection, a transfection mixture was prepared by mixing plasmid solution diluted in 250 μl of Opti-MEM (Life Technologies) and Lipofectamine® 2000 solution (2.5 μl) in 250 μl of Opti-MEM. For PEI transfection, a transfection solution was mixed by combining plasmid solution diluted in 100 μl of Opti-MEM and 4 μl of 1 mg ml⁻¹ PEI solution in 100 μl of Opti-MEM. Both Lipofectamine® 2000 and the PEI transfection gave almost identical transfection efficiency in the inventors' culture condition. The transfected cells were further incubated for one day before subjected to an assay as described below.

MDA-MB-231 cells (female origin) and PC-3 cells (male origin) were maintained in in RPMI 1640 (Nissui Pharmaceutical) supplemented with 5% fetal bovine serum and penicillin-streptomycin-glutamine. MDA-MB-231 cells and PC-3 cells were seeded in a 10-cm culture dish at cell density of 2×10⁵ cells ml⁻¹ in 10 ml of the media and cultured for one day in the incubator. Transfection was performed by using 20 μL of Lipofectamine® 2000 transfection reagent. The transfected cells were incubated for one day before subjected to the NanoBIT-RhoA assay as described below.

HN12 cells (female origin) and Cal27 cells (male origin), which were characterized as part of a head and neck cancer cell oncogenome effort (Martin et al., 2014) and obtained from this NIH cell collection, were maintained in DMEM supplemented with 10% FBS (Sigma-Aldrich).

### Method Details

### Plasmids

Only human GPCRs and human Gα subunits were used in this study. An open reading frame of each full-length GPCR was cloned into pCAGGS expression plasmid (a kind gift from Dr. Jun-ichi Miyazaki at Osaka University, Japan) or pcDNA3.1 expression plasmid. Except when otherwise specified, GPCR sequences were devoid of epitope tags. The GPCRs examined for this study (148 GPCRs) originated from a previous GPCR library (109 GPCRs) (Inoue et al., 2012) and an extended list of GPCR families (39 GPCRs). In their library, the inventors covered all of the members for selected GPCR families. The inventors note that there are 8 GPCRs (AGTR2, GPBAR1, GPER, GPR18, HTR5A, MC2R, NPBWR2 and PTGDR2) that were unresponsive in the chimeric G-protein-based TGFα shedding assay (data not shown) and thus were not included in the G-protein coupling dataset.

Full-length, untagged Gα subunits were cloned into the pCAGGS plasmid. Chimeric Gα subunits, in which the C-terminal 6 amino acids were substituted, were generated with PCR-amplified fragments using synthesized oligonucleotides encoding swapped C-terminal sequences. A C-terminally truncated Gα_{q} subunit, which lacked 7 amino acids (note that the −7 position is identical among all of the Gα subunits), was used as a negative control for the chimeric-G-protein-based TGFα shedding assay. Inserted sequences were verified by Sanger sequencing (Fasmac). Codon-optimized AP-TGFα cloned into the pCAGGS plasmid was used in this study. Amino acid sequences for the AP-TGFα construct are listed in SEQ ID Nos. 93-96 and the amino acid sequences for the chimeric-G-proteins are listed in 58 to 92.

M3D and M4D (Armbruster et al., 2007) were generated by introducing the two mutations (Y^{3.33}C and A^{5.46}G), which alter ligand specificity from ACh to CNO, in human CHRM3 (corresponding to Y149C and A239G) and CHRM4 (Y113C and A203G), respectively, by using an NEBuilder HiFi DNA Assembly system (New England Biolabs) and cloned into the pcDNA3.1 vector with N-terminal FLAG-epitope (DYKDDDDK) tag. ICL3-substituted M3D chimeras were constructed by the NEBuilder system with PCR-amplified fragments using synthesized oligonucleotides encoding swapped ICL3 sequences. Dual ICL3- and C-terminally-substituted M3D chimeras were generated by assembling PCR-amplified fragments with synthesized oligonucleotides for C-terminal sequences. The substituted ICL3 and C-terminus correspond to residues 778-1455 and 1633-1770 of the CHRM3 ORF. A coding sequence for M3D-Gₛ (Guettier et al., 2009) was human codon-optimized and gene-synthesized by Genscript and inserted into pcDNA3.1 with the N-terminal FLAG-epitope tag. Throughout the study, the inventors used the same N-terminally FLAG-tagged DREADD constructs for functional assays and expression analysis. Amino acid sequences used for suitable DREADDs are shown in SEQ ID Nos. 1 to 4 and 52 to 54.

For NanoBiT-G-proteins (see SEQ ID Nos. 5 to 48), the large fragment (LgBiT) of the NanoBiT luciferase (See SEQ ID. No. 49) was inserted into the helical domain of human Gα subunit (Gα-Lg) flanked by 15-amino acid flexible linkers (see SEQ ID No. 51) and the small fragment (SmBIT) (see SEQ ID No. 50) was N-terminally fused to human Gβ subunit (Sm-Gβ) or human Gγ subunit (Sm-Gγ) with the 15-amino acid linker. A coding sequence for the Gα-Lg was human codon-optimized and gene-synthesized by Genscript and inserted into pcDNA3.1 plasmid. To construct a coding sequence for the Sm-Gβ and the Sm-Gγ oligonucleotides encoding the N-terminal SmBiT-linker (Fasmac) and PCR-amplified fragment of full-length Gβ (Gβ₁, Gβ₃ or Gβ₅) or Gγ (Gγ₂ or Gyₜ₁) were assembled by using the NEBuilder system and cloned into the pCAGGS vector. Coding sequences for untagged Gβ₁ and Gγ₂ were inserted into pcDNA3.1 vector. Coding sequences for RIC8A and RIC8B (isoform 2) were cloned into pCAGGS vector.

For generation of the inventive NanoBiT-RhoA sensor, the inventors replaced firefly luciferase fragments of previously described RhoA constructs (Leng et al., 2013) with the NanoBiT fragments. Specifically, LgBIT and SmBIT were N-terminally fused to human RhoA (residues 2-193) and the GTPase-binding domain (GBD) of human PKN1 (residues 13-112), a RhoA effector, respectively, with the 15-amino acid linker. A coding sequence for RhoA and PKN1-GBD was human codon-optimized and gene-synthesized by Genscript and inserted into the pCAGGS plasmid by following a similar method as described in the NanoBiT-G-protein construction. Amino acid sequences for the NanoBIT-RhoA constructs (Lg-RhoA and Sm-PKN1) are listed in SEQ ID Nos. 55 and 56.

Similarly, to construct the inventive NanoBIT-IP3 sensor, the inventors exchanged firefly luciferase fragments of a previously described IP3 construct (Ataei et al., 2013) with the NanoBiT fragments. Specifically, LgBIT and SmBIT were fused to N-terminus and C-terminus, respectively, of IP3-binding core domain (IBC) of human type 2 IP3 receptor (Gene symbol ITPR2; residues 225-604), flanked by the 15-amino acid linker. A coding sequence for ITPR2-IBC was human codon-optimized and gene-synthesized by Genscript, and inserted into the pCAGGS plasmid by following an above-described method. An amino acid sequence for the NanoBiT-IP₃ sensor (Lg-IP3R2-Sm) is listed in SEQ ID No. 57.

### TGFα Shedding Assay

The TGFα shedding assay was performed as described previously (Inoue et al., 2012) with minor modifications. Plasmid transfection was performed in a 6-well plate with a mixture of 500 ng AP-TGFα-encoding plasmid, 200 ng GPCR-encoding plasmid with or without 100 ng Gα-encoding plasmid (per well, hereafter). After 1-day culture, the transfected cells were harvested by trypsinization, pelleted by centrifugation at 190 g for 5 min and washed once with Hank's Balanced Salt Solution (HBSS) containing 5 mM HEPES (pH 7.4). After centrifugation, the cells were resuspended in 6 ml of the HEPES-containing HBSS. The inventors note that trypsinization and following washing procedure resulted in higher signal-to-background TGFα shedding response as compared with harvesting cells without trypsin (EDTA only). The cell suspension was seeded in a 96-well culture plate (cell plate) at a volume of 90 μl (per well hereafter) and incubated for 30 min in a 5% CO₂ incubator at 37° C.. The cells were treated with a GPCR ligand (10×, diluted in HBSS containing 5 mM HEPES (pH 7.4) and 0.01% (w/v) bovine serum albumin (BSA, fatty acid-free and protease-free grade; Serva)). After spinning the cell plates, conditioned media (80 μl) was transferred to an empty 96-well plate (conditioned media (CM) plate). AP reaction solution (10 mM p-nitrophenylphosphate (p-NPP), 120 mM Tris-HCl (pH 9.5), 40 mM NaCl, and 10 mM MgCl₂) was dispensed into the cell plates and the CM plates (80 μl). Absorbance at 405 nm (Abs₄₀₅) of the plates was measured, using a microplate reader (SpectraMax 340 PC384, Molecular Devices), before and after 1-h or 2-h incubation at room temperature. Ligand-induced AP-TGFα release was calculated as described previously. Unless otherwise noted, spontaneous AP-TGFα release signal, which varies from 8-30% of total AP-TGFα expression depending on transfected conditions, was subtracted from ligand-induced AP-TGFα release signal. Using the Prism 7 software (GraphPad Prism), the AP-TGFα release signals were fitted to a four-parameter sigmoidal concentration-response curve, from which EC₅₀ and Eₘₐₓ values were obtained.

### Calculation of G-Protein Coupling Score

The inventors used a factor known as the relative intrinsic activity (RAi) (Ehlert et al., 1999) to calculate scores for G-protein coupling. For each sigmoidal curve of chimeric Gα-expressed condition, the inventors divided a maximal response (Eₘₐₓ) by a potency (EC₅₀) and normalized an Eₘₐₓ/EC₅₀ value to a maximum value among 11 chimeric Gα curves. The resulting dimensionless, relative Eₘₐₓ/EC₅₀ (defined as RAi) parameter was then log (base 10) transformed to give Log RAi values used to quantify coupling. To minimize the occurrence of outliers arising from experimental variations especially for weak AP-TGFα release signal, the inventors set two thresholds. As a first threshold, a Gα chimera condition in which Eₘₐₓ Was smaller than 3% AP-TGFα release or a concentration-response curve did not converge, was regarded as RAi value of 0. As a second threshold, RAi value smaller than 0.01 was set as 0.01. Thus, a Log RAi values range from −2 to 0 and for the bioinformatics analyses, the inventors used mean values of Log RAi (n=3-6).

### NanoBiT-G-Protein Dissociation Assay

Plasmid transfection was performed in a 6-well plate with a mixture of 100 ng Gα-Lg-encoding plasmid, 500 ng Sm-Gβ-encoding plasmid, 500 ng untagged Gγ₂-encoding plasmid, 200 ng GPCR-encoding plasmid with or without 100 ng RIC8-encoding plasmid (per well, hereafter). Unless otherwise stated, the combination of following plasmid mixtures was used: Gαₛ-Lg, Sm-Gβ₁, Gγ₂ and RIC8B for NanoBiT-Gₛ; Gαᵢ₁-Lg, Sm-Gβ₅ and Gγ₂ for NanoBiT-Gᵢ₁; Gαᵢ₂-Lg, Sm-Gβ₃ and Gγ₂ for NanoBiT-Gᵢ₂; Gαᵢ₃-Lg, Sm-Gβ₃ and Gγ₂ for NanoBiT-Gᵢ₃; Gdo-Lg, Sm-Gβ₁ and Gγ₂ for NanoBiT-Gₒ; Gα_{q}-Lg, Sm-Gβ₁, Gγ₂ and RIC8A for NanoBiT-G_{q}; Gα₁₂-Lg, Sm-Gβ₁, Gγ₂ and RIC8A for NanoBiT-G₁₂; Gα₁₃-Lg, Sm-Gβ₁, Gγ₂ and RIC8A for NanoBiT-G₁₃. After 1-day culture, the transfected cells were harvested with 1 mL of 0.53 mM EDTA-containing Dulbecco's PBS (D-PBS), followed by addition of 2 mL the HEPES-containing HBSS. The cells were pelleted by centrifugation at 190 g for 5 min and resuspended in 2 mL of the 0.01% BSA- and 5 mM HEPES (pH 7.4)-containing HBSS (assay buffer). The cell suspension was seeded in a 96-well culture white plate (Greiner Bio-One) at a volume of 80 μl (per well hereafter) and loaded with 20 μl of 50 μM coelenterazine (Carbosynth) solution diluted in the assay buffer. After 2-h incubation with coelenterazine at room temperature, background luminescent signals were measured using a luminescent microplate reader (SpectraMax L, Molecular Devices). The inventors note that incubation time with coelenterazine can be shortened, but an effect of baseline drift should be taken into account (FIG. 14C-E). Test compound (6X, diluted in the assay buffer) was manually added to the cells (20 μl). Luminescent signals were measured 3-5 min after ligand addition and divided by the initial count. The ligand-induced signal ratio was normalized to that treated with vehicle. The consequent fold-change values were fitted to a four-parameter sigmoidal concentration-response described above.

NanoBiT-RhoA assay

Plasmid transfection in HEK293 cells was performed by using a mixture of 100 ng Lg-RhoA plasmid, 500 ng Sm-PKN1 plasmid and 200 ng GPCR plasmid (per well in a 6-well plate). For transfection in MDA-MB-231 cells and PC-3 cells, 1.5 μg Lg-RhoA plasmid and 7.5 μg of Sm-PKN1 plasmid were used (per 10-cm dish). The transfected cells were harvested, seeded in a white 96-well plate and loaded with 10 μM CTZ in the same manner described in the NanoBiT-G-protein dissociation assay. After measuring an initial luminescent signal, test compounds were added to the cells. Then, 3-5 min later, luminescent signals were measured and fold-change values were plotted as described above.

### NanoBiT-IP₃ Sensor Assay

Plasmid transfection was performed by using a mixture of 1 μg Lg-IP3R2-Sm plasmid and 200 ng GPCR plasmid (per well in a 6-well plate). The transfected cells were harvested, seeded in a white 96-well plate and loaded with 10 μM CTZ in the same manner described in the NanoBiT-G-protein dissociation assay. After measuring an initial luminescent signal, test compounds were added to the cells. Then, 5-10 min later, luminescent signals were measured and fold-change values were plotted as described above.

### NanoBiT-G_{q}-PLCβ Interaction Assay

Plasmid transfection was performed by using a mixture of 100 ng Gα-Lg-encoding plasmid, 500 ng Sm-PLCβ-encoding plasmid, 500 ng untagged Gβ₁-encoding plasmid, 500 ng untagged Gγ₂-encoding plasmid, 200 ng GPCR-encoding plasmid and 100 ng RIC8A-encoding plasmid (per well in a 6-well plate, hereafter). The transfected cells were harvested, seeded in a white 96-well plate and loaded with 10 μM CTZ in the same manner described in the inventive NanoBiT-G-protein dissociation assay. After measuring an initial luminescent signal, test compounds were added to the cells. Then, 5-10 min later, luminescent signals were measured and fold-change values were plotted as described above.

siRNA Transfection

Stealth siRNA duplexes against mRNA encoding Gα_{q}, Gα₁₁, Gα₁₂, Gα₁₃ and TACE (gene symbols, GNAQ, GNA11, GNA12, GNA13 and ADAM17, respectively) and Stealth negative control were purchased from Life Technologies. Target sequences and manufacturer's catalog numbers are as follows: GNAQ (#1), 5′-GGAGAGAGUGGCAAGAGUACGUUUA-3′, GNAQHSS104236; GNAQ (#2), 5′-CCCUUUGACUUACAAAGUGUCAUUU-3′, GNAQHSS104237; GNA11 (#1), 5′-CCGGCAUCAUCGAGUACCCUUUCGA-3′, GNA11HSS178464; GNA11 (#2), 5′-GCAUCAGUACGUCAGUGCCAUCAAG-3′, GNA11HSS104213; GNA12 (#1), 5′-CCAAGGGAAUUGUGGAGCAUGACUU-3′, GNA12-HSS178466; GNA12 (#2), 5′-CCAUCGUCAACAACAAGCUCUUCUU-3′, GNA12MSS204749; GNA13 (#1), 5′-CAGAAGCCCUUAUACCACCACUUCA-3′, GNA13-HSS173827; GNA13 (#2), 5′-GCAGCCCAAGGAAUGGUGGAAACAA-3′, GNA13-HSS116479; ADAM17, 5′-CAGAAUCGUGUUGACAGCAAAGAAA-3′, ADAM17-HSS186181. SiRNA constructs for the GNA12 (#1), the GNA13 (#1) and the ADAM17 genes were described previously and validated (Inoue et al., 2012).

HEK293 cells were seeded in a 6-well culture plate at cell density of 1×10⁵ cells ml⁻¹ in 2 ml of the complete DMEM and incubated for 1 day. Transfection of siRNA transfection was performed by using Lipofectamine® RNAiMAX (Thermo Fisher Scientific) according to the manufacturer instructions (final siRNA concentration of 10 nM and 2 μL (per well in a 6-well plate) of Lipofectamine® RNAiMAX). After 1-day incubation, media were replaced and transfection of plasmids encoding AP-TGFα and GPCR was performed as described above. The resulting cells were subjected to the TGFα shedding assay.

### Quantitative Real-Time PCR Analysis

Total RNA from siRNA-transfected HEK293 cells was prepared using a GenElute Mammalian Total RNA Miniprep Kit (Sigma-Aldrich). Total RNA was reverse-transcribed using High-Capacity cDNA RT Kits (Applied Biosystems) according to manufacturer instructions. Real-time quantitative PCRs were performed with SYBR Premix Ex Taq (Takara Bio) and monitored by ABI Prism 7300 (Applied Biosystems). Standard plasmids ranging from 102-108 copies per well were used to quantify the absolute number of transcripts of cDNA samples. The numbers of transcripts were normalized to the number of GAPDH in the same sample and expressed as relative values to that in control siRNA-transfected cells.

Primers were as follows:

GNAQ,
5′-ACCGAATGGAGGAAAGCAAGG-3′
and
5′-CATCTCTCTGGGGTCCATCATATTC-3′;
GNA11,
5′-CAGCGAATACGACCAAGTCC-3′
and
5′-ACCAGGGGTAGGTGATGATG-3′;
GNA12,
5'-GAGGGATTCTGGCATCAGG-3′
and
5′-CGATCCGGTCCAAGTTGTC-3′;
GNA13,
5′-CCTGGATAACTTGGATAAACTTGG-3′
and
5′-TTCATGGATGCCTTTGGTG-3′;
GAPDH,
5'-GCCAAGGTCATCCATGACAACT-3′
and
5′-GAGGGGCCATCCACAGTCTT-3′.

### Western Blot

The parental HEK293 cells and a panel of the G-protein-KO HEK293 cells (ΔG_{q}, ΔG₁₂ and ΔG_{q}/ΔG₁₂ cells) in growth phase were harvested and approximately 1×10⁶ cells were lysed in 500 μL of SDS-PAGE sample buffer (62.5 mM Tris-HCl (pH 6.8), 50 mM dithiothreitol, 2% SDS, 10% glycerol and 4 M urea) containing 1 mM EDTA and 1 mM phenylmethylsulfonyl fluoride. Cell lysates were homogenized with a hand-held ultrasonic homogenizer (Microtech) and proteins were denatured at 95° C. for 5 min. The lysates were loaded and separated on a 12.5% polyacrylamide SDS-gel. After electrophoresis, the gel was blotted to a nitrocellulose membrane. The blotted membrane was blocked with 5% skim milk-containing blotting buffer (10 mM Tris-HCl (pH 7.4), 190 mM NaCl and 0.05% Tween 20), immunoblot with primary (1 μg ml⁻¹) and secondary antibodies (1:2000 dilution). Primary antibodies used in this study were anti-Gα_{q} antibody (goat polyclonal; Abcam, ab128060), anti-Gα₁₁ antibody (mouse monoclonal, clone D-6; Santa Cruz Biotechnologies, sc-390382), anti-Gα₁₃ antibody (rabbit monoclonal, clone EPR5436; Abcam, ab128900) and anti-α-tubulin antibody (mouse monoclonal, clone DM1A; Santa Cruz Biotechnologies, sc-32293). The inventors note that by using cell lysates overexpressing Gα subunits, the anti-Gα_{q} antibody and the anti-Gα₁₃ antibody were validated to be specific, but the anti-Gα₁₁ antibody reacted with both Gα_{q} and Gα₁₁ (data not shown), and thus the inventors labeled immuno-reactive bands as Gα_{q/11}. Secondary antibodies were conjugated with horseradish peroxidase (HRP) and were anti-goat IgG antibody (American Qualex, A201PS), anti-mouse IgG (GE Healthcare, NA9310) and anti-rabbit IgG (GE Healthcare, NA9340). Membrane were soaked with a commercial chemiluminescent reagent (ImmunoStar® Zeta, FujiFilm Wako Pure Chemicals) or in-house reagent (100 mM Tris-HCl (pH 8.5), 50 mg ml⁻¹ Luminol Sodium Salt HG (FujiFilm Wako Pure Chemicals), 0.2 mM p-Coumaric acid and 0.03% (v/v) of H₂O₂), and a chemiluminescence image was acquired with a LAS-4000 (FujiFilm) and analyzed with Multi Gauge ver. 3.0 (FujiFilm).

### Flow Cytometry

Plasmid transfection was performed in a 12-well plate with volumes of 500 ng plasmid encoding N-terminally FLAG epitope-tagged GPCR with or without 250 ng Gα-encoding plasmid. The transfected cells were harvested by adding 300 μl of 0.53 mM EDTA-containing D-PBS, followed by 300 μl of 5 mM HEPES (pH 7.4)-containing Hank's Balanced Salt Solution (HBSS). The cell suspension was dispensed in a 96-well V-bottom plate (200 μl per well, two wells per sample). After centrifugation at 700 g for 1 min, the cells were washed once with D-PBS and pelleted. Cell pellets were suspended in 2% goat serum- and 2 mM EDTA-containing D-PBS (blocking buffer; 100 μl per well) and incubated for 30 min on ice. After centrifugation at 700 g for 1 min, the cells were stained with anti-FLAG epitope tag monoclonal antibody (Clone 1E₆, FujiFilm Wako Pure Chemicals; 10 μg ml⁻¹ in the blocking buffer; 50 μl per well) for 30 min on ice. After rinse with D-PBS, cells were labeled with a goat anti-mouse IgG secondary antibody conjugated with Alexa Fluor 488 (Thermo Fisher Scientific; 10 μg ml⁻¹ dilution in the blocking buffer; 25 μl per well) for 15 min on ice. The cells were washed once with D-PBS, resuspended in 100 μl of 2 mM EDTA-containing-D-PBS and filtered through a 40 μm filter. The fluorescently labeled cells (approximately 20,000 cells per sample) were analyzed by an EC₈₀₀ flow cytometer (Sony). Fluorescent signal derived from Alexa Fluor 488 was recorded in an FL1 channel and flow cytometry data were analyzed by a FlowJo software (FlowJo). Values of mean fluorescence intensity (MFI) were used for quantification.

### GloSensor CAMP Assay

Plasmid transfection was performed in a 6-well plate with a mixture of 1 μg Glo-22F cAMP biosensor-encoding pCAGGS plasmid (gene synthesized with codon optimization by Genscript), 200 ng AVPR2-encoding plasmid and 100 ng of Gαₛ-Lg-encoding plasmid or native Gαₛ-encoding plasmid. After 1-day incubation, the transfected cells were harvested with 0.53 mM EDTA-containing D-PBS, centrifuged at 190 g for 5 min and suspended in 0.01% BSA- and 5 mM HEPES (pH 7.4)-containing HBSS (vehicle; 0.6 ml per well). The cells were seeded in a half-area white 96-well plate (Greiner Bio-one; 30 μL per well) and loaded with D-luciferin potassium solution (10 μL of 8 mM solution per well; FujiFilm Wako Pure Chemical, Japan). After 2 h incubation in the dark at room temperature, the plate was read for its initial luminescent count (integration time of 1 s per well; Spectramax L, Molecular Devices, Japan). The cells were treated with vehicle, arginine vasopressin (Peptide Institutes, Japan) or 10 μM forskolin (FujiFilm Wako Pure Chemical, Japan) (10 μL of 5×solution per well). Kinetics values were measured on the plates for 20 min and expressed as fold-change values. To obtain a concentration-response curve, fold-change luminescent signals at 10-min after compound addition were normalized to that in forskolin-treated condition. Using the Prism 7 software (GraphPad Prism), the CAMP signals were fitted to a four-parameter sigmoidal concentration-response curve, from which EC₅₀ values were obtained.

For the chimeric Gₛ-based CAMP assay, ΔGₛ cells were transfected with a mixture of 1 μg Glo-22F plasmid, 200 ng GPCR plasmid and 100 ng chimeric Gαₛ plasmid containing the backbone of human Gαₛ subunit (short isoform, residues 1-374) and a substitution of C-terminal 6-amino acids. The transfected cells were harvested, seeded in the half-area 96-well plate, loaded with D-luciferin and stimulated with a GPCR ligand in the same manner as described above. Scores of G-protein coupling (RAi values) values were calculated as described in the TGFα shedding assay section.

### Active RhoA Pulldown Assay

HN12 cells and Cal27 cells were cultured to 50% confluency, and then serum starved overnight. To induce RhoA activation, cells were treated with 5 μM LPA, 1 μM Ang II, or 10 μM CP-55940 for 5 min. Active RhoA levels were measured using the RhoA Pull-Down Activation Assay Biochem Kit (bead pull-down format; Cytoskeleton) following the manufacturer instruction using a modified lysis buffer (50 mM Tris-HCl (pH 7.2), 500 mM NaCl, 10 mM MgCl₂, 0.1% SDS, 1% NP-40). Briefly, after stimulation, samples were lysed and protein concentrations were quantified using DC Protein Assay (BioRad). Samples were adjusted to the same concentration with lysis buffer and 500 μg of each protein lysate was added to 15 μL GST-tagged Rhotekin-RBD bound to Sepharose beads. Samples were incubated while rocking at 4° C. for 1.5 h. Beads were then washed, eluted in Laemmli sample buffer, and analyzed by western blot using a mouse monoclonal anti-RhoA antibody (Cytoskeleton).

### Ca²⁺ Mobilization Assay

Plasmid transfection was performed in the parental, ΔG_{q} and ΔG12 HEK293 cells by using 5 μg GPCR plasmid (AGTR1 or EDNRA; 5 μg per 10-cm culture dish). After one-day incubation, the transfected cells were harvested with trypsinization. After centrifugation, the cells were suspended in serum-free DMEM at a cell concentration of 5×10⁵ cells ml⁻¹, and 40 μl (per well hereafter) of the cell suspension seeded in a half-area, clear-bottom black plate. The cells were further incubated in the incubator for one day. After loading 40 μl of a Ca²⁺ indicator (FLIPR Calcium 5 Assay Kit, Molecular Devices) according to manufacturer instructions in the presence of 2.5 mM probenecid for 1 h in the incubator, the cell plate was placed in a fluorescence microplate reader (FlexStation 3, Molecular Devices).

Fluorescent signal was measured with automated pipetting of test ligands (20 μL of 5X compounds). Fluorescent signals from 40 to 55 sec after ligand addition were averaged and normalized to an initial count and expressed as a relative value to vehicle treatment.

Comparison of Data from the Chimeric G-Protein-Based Assay with Known Couplings

The inventorsperformed Receiver Operating Characteristic (ROC) analysis to compare the chimeric G-protein-based TGFα shedding assay results to primary or secondary couplings from GtoPdb (Harding et al., 2018), defined as binary classifiers. The inventors defined the optimal LogRAi cutoff as that maximizing the True Positive Rate (TPR, or sensitivity) while minimizing the True Negative Rate (TNR, or 1-specificity). The inventors defined positives as GtoPdb couplings reported in at least 3 references, and negatives as the couplings that were never reported for these more studied receptors. The inventors obtained a value close to −1 as the optimal LogRAi cutoff considering all G-proteins altogether (FIG. 15), which the inventors then considered as a lower and upper confidence bound for positively and negatively coupled receptors.

### Sequence-Based Coupling Determinant Features

The inventorsfirst generated a multiple sequence alignments (MSAs) of the 144 Class A GPCR sequences using HMMalign from the HMMer package (Eddy, 1998), using the 7tm_1 Pfam (Finn et al., 2016) Hidden Markov Model (HMM). The inventors subdivided the pool of receptor sequences into positively and negatively coupled to a given G-protein using the optimal LogRAi cutoff as a lower and upper bound. These sub-alignments were used to build corresponding HMM profiles through hmmbuild (http://www.hmmer.org/), leading to 22 models (coupled vs. uncoupled for 11 G-proteins).

From coupled and uncoupled HMM profiles for each G-protein, the inventors then extracted alignment positions present in both HMM models and showing statistically different distributions (Wilcoxon's signed-rank test; p-value <=0.05) of the 20 amino acid bit scores (FIG. 4A). The inventors also considered those alignment positions with consensus columns (i.e. those having a fraction of residues, as opposed to gaps, equal or greater than the hmmbuild's symfrac parameter, using default value of 0.5) present in either of HMM models. In details, if a consensus column was present only in the HMM profile of either the coupled or uncoupled groups, the inventors labelled it as insertion or deletion, respectively. As additional features, the inventors also included length and amino acid composition of the N- and C-termini (N-term and C-term) and the extra- and intra-cellular loops (ECLs and ICLs). For every G-protein, only statistically significant (p-value <0.05; Wilcoxon's rank-sum test) features were considered.

To identify each positions within the alignment, the inventors employed the Ballesteros/Weinstein scheme (Weinstein, 1995), using the consensus secondary structure from the 7tm_1 HMM model to number residues within helices in a consecutive way. Most conserved positions within each helix were defined according to GPCRDB (http://www.gpcrdb.org) (Isberg et al., 2017). The inventors adjusted the B/W numberings for TM6, which they started at position 6.25 (domain position 200) instead of 6.31 (domain position 206), according to visual inspections of recent G protein-GPCRs complexes. If a position lies on an extra-7TM region (e.g. ECLs or ICLs), the inventors use the corresponding label plus the corresponding Pfam domain consecutive numbering in parenthesis.

### G-Protein Coupling Predictor

The inventorsimplemented a predictor for G-protein coupling by using a logistic regression classifier, or Log-reg classifier, available from the scikit-learn package (http://scikit-learn.org) (Pedregosa F, 2011) The possible outcomes in log-reg are modeled using a logistic function, with L1 or L2 based regularization. In this study the inventors used L2 penalized form of log-reg. The target value is expected to be a linear combination of the given features. This property of log-reg can also be exploited to study the weights of its features.

As an optimization problem, binary class L2 penalized logistic regression minimizes the following cost function:

$${min}_{w , c} \frac{1}{2} {w}^{T} w + C {\sum }_{i = 1}^{n} log ( exp ( - {y}_{i} ( {X}_{i}^{T} w + c ) ) + 1 ) \qquad 1 )$$

where X denotes a vector of feature variables, w ∈R\hat{ }n is the weight vector, c ∈R\hat{ }n is the intercept, C is inverse of regularization strength (positive float), y takes values in {−1, 1} at trial i and n is the number of trials conducted.

The inventorsused the liblinear method as the optimization algorithm as shown to be optimal for relatively small datasets (https://www.csie.ntu.edu.tw/~cjlin/liblinear/).

### Training and Cross Validation

The inventorsused 7TM domain positions and compositional features for the ICL3 and C-term, which prevail over other extra-7TM domain features, to create a training matrix. In case of significant positional features, two-bit scores (derived from the positive and negative HMMs for a given G-protein) are returned for the corresponding amino acid found at a given position in the input GPCR sequence (FIG. 4A). In case a position was found to be present in either positive or negative HMMs, the single bit score, derived from the respective HMM, was returned. If for any GPCR, no amino acid was present at the given position, it was assigned the highest bit scores from the both models, implying the least conserved scores.

All the features were scaled to the range [0,1]. Feature scaling aids not only in converging the algorithm faster but also helps in assessing the feature relevance (Dou et al., 2012). A grid search was performed over a stratified 5-fold cross validation (CV) to select the best value of C (inverse of the regularization strength) over a range of [1e-02, 1e05]. In a stratified 5-fold CV, the training matrix is divided randomly into 5 equal sub-matrices, preserving the ratio of positive (coupling) and negative (non-coupling) GPCRs. During each fold, one of the sub matrix is treated as the validation set and the remaining four as the training set.

The inventorsassessed the performance of the inventors' predictor using standard metrics (MCC, ACC, PRE, REC, SPE, AUC, F1M). The parameters showing the best Area Under the Curve (AUC) of the Receiver Operating Curve (ROC) were chosen to create models for every G-protein.

The number of positive (coupling) GPCRs were either more (eg: in GNAI1/3) or less (eg: in GNAS) than the number of negative (non-coupling) GPCRs. Such an imbalance would make the predictor biased to any one of the two classes. In order to counter this problem, the parameter class_weight was set to balanced in the log-reg classifier function. By default, all the classes have same weight. However, by setting the class_weight as balanced, the values of the column with classes (coupling/uncoupling) are used to automatically adjust the weights inversely proportional to their frequencies in the training matrix. To ensure minimal variance due to random division of the training matrix during the cross validation, the aforementioned experiment was repeated ten times for every G-protein group and the standard deviation was recorded. The feature weights were extracted as described elsewhere (Dou et al., 2012) from the trained models and are critical to understand the relative importance of different features (FIG. 4).

Besides performing the above-said steps at LogRAi cutoff of −1.0, the inventors also created models at LogRAi cutoffs −0.5 and −0.1. As it can be seen in FIG. 15A, for most of the G-proteins, −1.0 turns out to be the best LogRAi cutoff during cross-validation (using MCC as the selection criteria).

### Randomized Training Test

In order to assess over-fitting, the inventors performed a randomization test (Sgourakis et al., 2005b). For every G-protein, the original labels of the training matrix were replaced with randomly determined labels, while preserving the ratio of number of positive (coupling) and negative (non-coupling) GPCRs. Performance using the randomization training set was lower than that of actual training set, implying that the inventors' strategy is insensitive to the data training set.

### Test Set to Benchmark Predictor Performance

To benchmark the inventive method and compare it with Pred Couple (Sgourakis et al., 2005b), a web-server available to predict GPCR-G-protein coupling, the inventors extracted all the GPCRs from GtoPdb that are present in neither the chimeric G-protein-based TGFα shedding assay nor in Pred Couple's training set, thus obtaining a list of 86 unseen GPCRs. As mentioned above, one of the major limitations of GtoPdb is the absence of a definite true negative set, thus, the best measure to compare the inventive predictor with that of Pred Couple is recall, also known as sensitivity or the true positive rate. Since both GtoPdb and Pred Couple provide coupling information at the G-protein family level, the inventors combined the performance of individual G-protein predictors based on their families to compare the performance of the inventive method of the first inventive aspect with Pred-Couple. For example: if a given GPCR was predicted to couple to at least one of the G-proteins of a family, it was annotated as coupling to that G-protein family. The combinations of the inventive predictors at the family level outperformed Pred-Couple over the test set. The individual G-protein predictors' and their combined (G-protein family level) performance over the Test set at different LogRAi cutoffs are reported in FIGS. 15A and 15C, respectively.

To further check the predictor's performance, the inventors trained and tested an additional predictor using exactly the same procedure as reported above using GtoPdb coupling information instead of the TGFα shedding assay (FIG. 4A).

### Functional Classification of Coupling Features Through 3D Structure Analysis.

The inventorsidentified functional positions as those mediating inter- and intra-molecular contacts, i.e. whenever at least one pair of atoms, from either a residue-residue or residue-ligand interface, was found spatially closer than 5 Å. The inventors analysed 246 3D structures, representing 51 members of the GPCR Class A (PFAM: 7tm_1) family using PDB-Swissprot-PFAM correspondences available from SIFT (as of July 2018) (Velankar et al., 2013).

To define GPCR-ligand contact sites, the inventors restricted their analysis only to GPCR putative ligands as defined in GtoPd (Harding et al., 2018). The inventors performed similarity searches between GtoPdb and PDB ligands using topological fingerprints from RDKit (http://www.rdkit.org/) generated from SMILES descriptors and the inventors considered only the best matching GtoPdb ligand for a given PDB component. All the protein residues mediating contacts were mapped to protein sequence position using alignments between Uniprot canonical sequences and corresponding PDB generated through Blast (Altschul et al., 1990). Note that through this procedure the inventors considered contacts mediated by the equivalent residues from different structures only once, thus avoiding overcounting due to PDB redundancy. The inventors then mapped the amino acids found in contact with putative ligands on the PFAM multiple sequence alignments (MSA). Based on available GPCR-G-protein complexes (PDB ID: 3SN6, 5G53, 6D9H, 6GDG, 6DDE, 6DDF, 6CMO and 6D9H) the inventors similarly identified the residues forming the receptor-G-protein interaction interface by using a distance cutoff of 6.5 Å to define atom-pairs forming inter-residue contacts.

Similarly to methods employed to decipher the activation mechanisms of GPCRs and other signaling molecules, the inventors also inspected the network of intramolecular contacts using the same thresholds as above and they similarly mapped the identified positions on Class A 7TM MSA. They then defined a consensus contact network by considering the sequence positions (nodes) found in contact (edges) in at least 50% of the analyzed sequences. They performed network analysis through igraph (Csardi and T., 2006), defining as hub nodes having a degree of at least 4. The inventors generated functional state consensus networks by grouping available structures using ligand classification from GtoPdb (i.e. agonist or antagonist/inverse agonist) or functional classification directly available from the protein databank (i.e. active or inactive), thus defining active-like (i.e. agonist-bound/active) or inactive-like (i.e. antagonist-bound/inactive) states. Structures where this classification was not possible were discarded.

The inventors calculated the shortest paths connecting positions forming the consensus ligand and G-protein binding interfaces within active- and inactive-like networks through the Dijkstra algorithm (Dijkstra, 1959) from igraph. Active-state specific shortest paths were defined as those characterized by having either endpoints, or intermediate connectivity residues, exclusive to active-like state contact network.

### G12-Coupled DREADD Chimeric Sequences Predictions

In order to inventively predict mutant sequences with enhanced G12/13 coupling capabilities, the inventors started from the available DREADD coupled with G_{q/11} (M3D) and G_{i/o} (M4D) (Armbruster et al., 2007). They generated chimeric sequences by swapping on these backbones the ICL3 and C-term sequence stretches derived from each receptor of the chimeric G-protein-based assay panel (148 GPCRs). The inventors first aligned the receptor sequences, including M3D and M4D, to the PFAM 7tm_1 HMM model. They defined ICL3 as the MSA region comprised within HMM positions 173-205, and the C-term as the MSA portion starting after 7tm_1 HMM end (i.e. position 268). They then created hM3D and hM4D chimeras by exchanging their ICL3 and C-term sequences with the corresponding sequences from each receptor testing in the chimeric G-protein-based TGFα shedding assay. They generated 296 chimeric sequences by swapping the ICL3 alone or in combination with the C-termini.

The inventors then predicted the coupling probability to GNA12/GNA13 for each chimeric sequence, ranking them according to their relative coupling probability (i.e. ΔPred_Coup=Pred_Coup^{DREADD_MUT}−Pred_Coup^{DREADD}). The inventors selected the top 10 chimeric sequences for experimental validation.

### Quantification and Statistical Analysis

Statistical analyses were performed using GraphPad Prism 7 software and methods are described in the legends of the figures. In flow cytometry experiments, approximately 20,000 cells were measured for their fluorescent signals and data were analyzed by FlowJo software. Mean fluorescent intensity was used for quantification of cell surface GPCR expression. Representation of symbols and error bars is described in the ligands. Symbols are either mean values of indicated numbers of independent experiments or datapoint from single experiment. Error bars denote SEM or SD. Concentration-response curves were fitted to all data by the Nonlinear Regression: Variable slope (four parameter) in the Prism 7 tool. Liner regression and representation of 90% confidence bands were performed by the Prism 7 tool. For multiple comparison analysis in the flow cytometry data and G12-DREADD generation, two-way ANOVA and following Dunnet's test and Sidak's test, respectively, was used.

### Data and Software Availability

The Python code used for the predictor is available on GitHub (https://github.com/raimondifranc/gpcr_coupling_predictor)

### CITED REFERENCES

Altschul, S. F., Gish, W., Miller, W., Myers, E. W., and Lipman, D. J. (1990). Basic local alignment search tool. J Mol Biol 215, 403-410.
Armbruster, B. N., Li, X., Pausch, M. H., Herlitze, S., and Roth, B. L. (2007). Evolving the lock to fit the key to create a family of G protein-coupled receptors potently activated by an inert ligand. Proc Natl Acad Sci USA 104, 5163-5168.
Ataei, F., Torkzadeh-Mahani, M., and Hosseinkhani, S. (2013). A novel luminescent biosensor for rapid monitoring of IP3 by split-luciferase complementary assay. Biosens Bioelectron 41, 642-648.
Capper, M. J., and Wacker, D. (2018). How the ubiquitous GPCR receptor family selectively activates signalling pathways. Nature 558, 529-530.
Chen, P., Zuo, H., Xiong, H., Kolar, M. J., Chu, Q., Saghatelian, A., Siegwart, D. J., and Wan, Y. (2017). Gpr132 sensing of lactate mediates tumor-macrophage interplay to promote breast cancer metastasis. Proc Natl Acad Sci USA 114, 580-585.
Csardi, G., and T., N. (2006). The igraph software package for complex network research. InterJournal Complex Systems.
Denker, B. M., Schmidt, C. J., and Neer, E. J. (1992). Promotion of the GTP-liganded state of the Go alpha protein by deletion of the C terminus. J Biol Chem 267, 9998-10002.
Devost, D., Sleno, R., Petrin, D., Zhang, A., Shinjo, Y., Okde, R., Aoki, J., Inoue, A., and Hebert, T. E. (2017). Conformational Profiling of the AT1 Angiotensin II Receptor Reflects Biased Agonism, G Protein Coupling, and Cellular Context. J Biol Chem 292, 5443-5456.
Dijkstra, E. (1959). A note on two problems in connexion with graphs. Numerische Mathematik 1, 269-271.
Dixon, A. S., Schwinn, M. K., Hall, M. P., Zimmerman, K., Otto, P., Lubben, T. H., Butler, B. L., Binkowski, B. F., Machleidt, T., Kirkland, T. A., et al. (2016). NanoLuc Complementation Reporter Optimized for Accurate Measurement of Protein Interactions in Cells. ACS chemical biology 11, 400-408.
Dorsam, R. T., and Kunapuli, S. P. (2004). Central role of the P2Y12 receptor in platelet activation. J Clin Invest 113, 340-345.
Dou, Y., Wang, J., Yang, J., and Zhang, C. (2012). L1pred: a sequence-based prediction tool for catalytic residues in enzymes with the L1-logreg classifier. PLOS One 7, e35666.
Eddy, S. R. (1998). Profile hidden Markov models. Bioinformatics 14, 755-763.
Ehlert, F. J., Griffin, M. T., Sawyer, G. W., and Bailon, R. (1999). A simple method for estimation of agonist activity at receptor subtypes: comparison of native and cloned M3 muscarinic receptors in guinea pig ileum and transfected cells. J Pharmacol Exp Ther 289, 981-992.
Finn, R. D., Coggill, P., Eberhardt, R. Y., Eddy, S. R., Mistry, J., Mitchell, A. L., Potter, S. C., Punta, M., Qureshi, M., Sangrador-Vegas, A., et al. (2016). The Pfam protein families database: towards a more sustainable future. Nucleic Acids Res 44, D279-285.
Flock, T., Ravarani, C. N. J., Sun, D., Venkatakrishnan, A. J., Kayikci, M., Tate, C. G., Veprintsev, D. B., and Babu, M. M. (2015). Universal allosteric mechanism for Galpha activation by GPCRs. Nature 524, 173-179.
Gales, C., Rebois, R. V., Hogue, M., Trieu, P., Breit, A., Hebert, T. E., and Bouvier, M. (2005). Real-time monitoring of receptor and G-protein interactions in living cells. Nat Methods 2, 177-184.
Guettier, J. M., Gautam, D., Scarselli, M., Ruiz de Azua, I., Li, J. H., Rosemond, E., Ma, X., Gonzalez, F. J., Armbruster, B. N., Lu, H., et al. (2009). A chemical-genetic approach to study G protein regulation of beta cell function in vivo. Proc Natl Acad Sci USA 106, 19197-19202.
Harding, S. D., Sharman, J. L., Faccenda, E., Southan, C., Pawson, A. J., Ireland, S., Gray, A. J. G., Bruce, L., Alexander, S. P. H., Anderton, S., et al. (2018). The IUPHAR/BPS Guide to PHARMACOLOGY in 2018: updates and expansion to encompass the new guide to IMMUNOPHARMACOLOGY. Nucleic Acids Res 46, D1091-D1106.
Hauser, A. S., Attwood, M. M., Rask-Andersen, M., Schioth, H. B., and Gloriam, D. E. (2017). Trends in GPCR drug discovery: new agents, targets and indications. Nat Rev Drug Discov 16, 829-842.
Hauser, A. S., Chavali, S., Masuho, I., Jahn, L. J., Martemyanov, K. A., Gloriam, D. E., and Babu, M. M. (2018). Pharmacogenomics of GPCR Drug Targets. Cell 172, 41-54 e19.
Herroeder, S., Reichardt, P., Sassmann, A., Zimmermann, B., Jaeneke, D., Hoeckner, J., Hollmann, M. W., Fischer, K. D., Vogt, S., Grosse, R., et al. (2009). Guanine nucleotide-binding proteins of the G12 family shape immune functions by controlling CD4+ T cell adhesiveness and motility. Immunity 30, 708-720.
Horn, F., van der Wenden, E. M., Oliveira, L., AP, I. J., and Vriend, G. (2000). Receptors coupling to G proteins: is there a signal behind the sequence? Proteins 41, 448-459.
Inoue, A., Ishiguro, J., Kitamura, H., Arima, N., Okutani, M., Shuto, A., Higashiyama, S., Ohwada, T., Arai, H., Makide, K., et al. (2012). TGFalpha shedding assay: an accurate and versatile method for detecting GPCR activation. Nat Methods 9, 1021-1029.
Insel, P. A., and Ostrom, R. S. (2003). Forskolin as a tool for examining adenylyl cyclase expression, regulation, and G protein signaling. Cell Mol Neurobiol 23, 305-314.
Isberg, V., Mordalski, S., Munk, C., Rataj, K., Harpsoe, K., Hauser, A. S., Vroling, B., Bojarski, A. J., Vriend, G., and Gloriam, D. E. (2017). GPCRdb: an information system for G protein-coupled receptors. Nucleic Acids Res 45, 2936.
Kihara, Y., Maceyka, M., Spiegel, S., and Chun, J. (2014). Lysophospholipid receptor nomenclature review: IUPHAR Review 8. Br J Pharmacol 171, 3575-3594.
Leng, W., Pang, X., Xia, H., Li, M., Chen, L., Tang, Q., Yuan, D., Li, R., Li, L., Gao, F., et al. (2013). Novel split-luciferase-based genetically encoded biosensors for noninvasive visualization of Rho GTPases. PLOS One 8, e62230.
Martin, D., Abba, M. C., Molinolo, A. A., Vitale-Cross, L., Wang, Z., Zaida, M., Delic, N. C., Samuels, Y., Lyons, J. G., and Gutkind, J. S. (2014). The head and neck cancer cell oncogenome: a platform for the development of precision molecular therapies. Oncotarget 5, 8906-8923.
Muppidi, J. R., Schmitz, R., Green, J. A., Xiao, W., Larsen, A. B., Braun, S. E., An, J., Xu, Y., Rosenwald, A., Ott, G., et al. (2014). Loss of signalling via Galpha13 in germinal centre B-cell-derived lymphoma. Nature 516, 254-258.
O'Hayre, M., Inoue, A., Kufareva, I., Wang, Z., Mikelis, C. M., Drummond, R. A., Avino, S., Finkel, K., Kalim, K. W., DiPasquale, G., et al. (2016). Inactivating mutations in GNA13 and RHOA in Burkitt's lymphoma and diffuse large B-cell lymphoma: a tumor suppressor function for the Galpha13/RhoA axis in B cells. Oncogene 35, 3771-3780.
Patel, Y. M., Lordkipanidze, M., Lowe, G. C., Nisar, S. P., Garner, K., Stockley, J., Daly, M. E., Mitchell, M., Watson, S. P., Austin, S. K., et al. (2014). A novel mutation in the P2Y12 receptor and a function-reducing polymorphism in protease-activated receptor 1 in a patient with chronic bleeding. J Thromb Haemost 12, 716-725.
Pedregosa F, V. G., Gramfort A, Michel V, Thirion B, Grisel O, Blondel M, Prettenhofer P, Weiss R, Dubourg V, Vanderplas J, Passos A, Cournapeau D, Brucher M, Perrot M, Duchesnay E (2011). Scikit-learn: Machine Learning in Python. J Machine Learning Res 12, 2825-2830.
Rasmussen, S. G., DeVree, B. T., Zou, Y., Kruse, A. C., Chung, K. Y., Kobilka, T. S., Thian, F. S., Chae, P. S., Pardon, E., Calinski, D., et al. (2011). Crystal structure of the beta2 adrenergic receptor-Gs protein complex. Nature 477, 549-555.
Rodriguez, G. J., Yao, R., Lichtarge, O., and Wensel, T. G. (2010). Evolution-guided discovery and recoding of allosteric pathway specificity determinants in psychoactive bioamine receptors. Proc Natl Acad Sci USA 107, 7787-7792.
Sauliere, A., Bellot, M., Paris, H., Denis, C., Finana, F., Hansen, J. T., Altie, M. F., Seguelas, M. H., Pathak, A., Hansen, J. L., et al. (2012). Deciphering biased-agonism complexity reveals a new active AT1 receptor entity. Nat Chem Biol 8, 622-630.
Schrage, R., Schmitz, A. L., Gaffal, E., Annala, S., Kehraus, S., Wenzel, D., Bullesbach, K. M., Bald, T., Inoue, A., Shinjo, Y., et al. (2015). The experimental power of FR900359 to study Gq-regulated biological processes. Nat Commun 6, 10156.
Sgourakis, N. G., Bagos, P. G., and Hamodrakas, S. J. (2005a). Prediction of the coupling specificity of GPCRs to four families of G-proteins using hidden Markov models and artificial neural networks. Bioinformatics 21, 4101-4106.
Sgourakis, N. G., Bagos, P. G., Papasaikas, P. K., and Hamodrakas, S. J. (2005b). A method for the prediction of GPCRs coupling specificity to G-proteins using refined profile Hidden Markov Models. BMC Bioinformatics 6, 104.
Sievers, F., Wilm, A., Dineen, D., Gibson, T. J., Karplus, K., Li, W., Lopez, R., McWilliam, H., Remmert, M., Soding, J., et al. (2011). Fast, scalable generation of high-quality protein multiple sequence alignments using Clustal Omega. Mol Syst Biol 7, 539.
Stallaert, W., van der Westhuizen, E. T., Schonegge, A. M., Plouffe, B., Hogue, M., Lukashova, V., Inoue, A., Ishida, S., Aoki, J., Le Gouill, C., et al. (2017). Purinergic Receptor Transactivation by the beta2-Adrenergic Receptor Increases Intracellular Ca²⁺ in Nonexcitable Cells. Mol Pharmacol 91, 533-544.
Sugimoto, Y., and Narumiya, S. (2007). Prostaglandin E receptors. J Biol Chem 282, 11613-11617.
Suzuki, N., Hajicek, N., and Kozasa, T. (2009). Regulation and physiological functions of G12/13-mediated signaling pathways. Neurosignals 17, 55-70.
Thomsen, W., Frazer, J., and Unett, D. (2005). Functional assays for screening GPCR targets. Current opinion in biotechnology 16, 655-665.
Urban, D. J., and Roth, B. L. (2015). DREADDs (designer receptors exclusively activated by designer drugs): chemogenetic tools with therapeutic utility. Annu Rev Pharmacol Toxicol 55, 399-417.
Velankar, S., Dana, J. M., Jacobsen, J., van Ginkel, G., Gane, P. J., Luo, J., Oldfield, T. J., O'Donovan, C., Martin, M. J., and Kleywegt, G. J. (2013). SIFTS: Structure Integration with Function, Taxonomy and Sequences resource. Nucleic Acids Res 41, D483-489.
Violin, J. D., Crombie, A. L., Soergel, D. G., and Lark, M. W. (2014). Biased ligands at G-protein-coupled receptors: promise and progress. Trends Pharmacol Sci 35, 308-316.
Waterhouse, A. M., Procter, J. B., Martin, D. M., Clamp, M., and Barton, G. J. (2009). Jalview Version 2—a multiple sequence alignment editor and analysis workbench. Bioinformatics 25, 1189-1191.
Weinstein, J. B. a.H. (1995). Integrated methods for the construction of three-dimensional models and computational probing of structure-function relations in G protein-coupled receptors. Methods Neurosci 25, 366-428.
Wess, J., Nakajima, K., and Jain, S. (2013). Novel designer receptors to probe GPCR signaling and physiology. Trends Pharmacol Sci 34, 385-392.
Wettschureck, N., and Offermanns, S. (2005). Mammalian G proteins and their cell type specific functions. Physiol Rev 85, 1159-1204.
Wheeler, T. J., Clements, J., and Finn, R. D. (2014). Skylign: a tool for creating informative, interactive logos representing sequence alignments and profile hidden Markov models. BMC Bioinformatics 15, 7.
Wong, S. K. (2003). G protein selectivity is regulated by multiple intracellular regions of GPCRs. Neurosignals 12, 1-12.
Woodward, D. F., Jones, R. L., and Narumiya, S. (2011). International Union of Basic and Clinical Pharmacology. LXXXIII: Classification of Prostanoid Receptors, Updating 15 Years of Progress. Pharmacol Rev 63, 471-538.
Yabuki, Y., Muramatsu, T., Hirokawa, T., Mukai, H., and Suwa, M. (2005). GRIFFIN: a system for predicting GPCR-G-protein coupling selectivity using a support vector machine and a hidden Markov model. Nucleic Acids Res 33, W148-153.

1. An amino acid sequence for determining coupling or no coupling between G-protein and G-protein coupled receptor (GPCR) mediated by a GPCR ligand in a cell based assay, wherein the amino acid sequence is selected from
a Gα subunit of the chimeric G-protein, which comprises or consists of an amino acid sequence according to any one of SEQ ID Nos: 5 to 15 and 33 to 41 or an amino acid sequence having at least 90, 91, 92, 93, 94, 95, 96, 97, 98, 99% sequence identity to any one of SEQ ID Nos: 5 to 15 and 33 to 41, and/or
a Gβ subunit of the chimeric G-protein, which comprises or consists of an amino acid sequence according to any one of SEQ ID Nos: 16 to 20, and 42 or an amino acid sequence having at least 90, 91, 92, 93, 94, 95, 96, 97, 98, 99% sequence identity to any one of SEQ ID Nos: 16 to 20, and 42, and/or
a Gγ subunit of the chimeric G-protein, which comprises or consists of an amino acid sequence according to any one of SEQ ID Nos: 21 to 32, 43 and 44 or an amino acid sequence having at least 90, 91, 92, 93, 94, 95, 96, 97, 98, 99% sequence identity to any one of SEQ ID Nos: 21 to 32, 43 and 44, and/or
a chimeric RhoA GTPase, which comprises or consists of an amino acid sequence according to SEQ ID No: 55 or an amino acid sequence having at least 90, 91, 92, 93, 94, 95, 96, 97, 98, 99% sequence identity to SEQ ID No: 55, and/or
a chimeric PKC-related serine/threonine-protein kinase N1 (PKN1), which comprises or consists of an amino acid sequence according to SEQ ID No: 56 or an amino acid sequence having at least 90, 91, 92, 93, 94, 95, 96, 97, 98, 99% sequence identity to SEQ ID No: 56, and/or
a chimeric inositole triphosphate receptor (IP3R), which is based on the inositol triphosphate receptor 2 (IP3R2) and comprises or consists of an amino acid sequence according to SEQ ID No: 57 or an amino acid sequence having at least 90, 91, 92, 93, 94, 95, 96, 97, 98, 99% sequence identity to SEQ ID No: 57 and/or
the chimeric 1-phosphatidylinositol-4,5-bisphosphate phospholipase Cbeta PLCβ, which comprises or consists of an amino acid sequence according to SEQ ID Nos: 45 to 48 or an amino acid sequence having at least 90, 91, 92, 93, 94, 95, 96, 97, 98, 99% sequence identity to SEQ ID Nos: 45 to 48.

2. An amino acid sequence for determining coupling or no coupling between G-protein and G-protein coupled receptor (GPCR) mediated by a GPCR ligand in a cell based assay as claimed in claim 1, wherein the GPCR is a G12-specific G-protein coupled receptor (GPCR) responding to a ligand and comprises or consists of an amino acid sequence according to SEQ ID Nos: 2, 3, or 4 or an amino acid sequence having at least 94, 95, 96, 97, 98, 99% sequence identify to SEQ ID nos: 2, 3 or 4.

3. A computer-implemented method for determining a probability of coupling or no coupling between a G-protein and a G-protein coupled receptor (GPCR), wherein the method comprises or consists of the following steps:
a. Providing amino acid sequence data and/or three dimensional (3D) structural data of:
i. one or more G-proteins and one or more GPCRs known to couple as a G-protein/GPCR complex and
ii. one or more G-proteins and one or more GPCRs known to not couple as a G-protein/GPCR complex,
b. Statistically aligning the amino acid sequence and/or the 3D structural data of the G-protein with the GPCR of the respective coupled or uncoupled G-protein/GPCR complex provided in step a) in order to determine one or more amino acid residues and/or one or more structural composition features found to be statistically significantly associated with a coupled G-protein/GPCR complex or with an uncoupled G-protein/GPCR complex, and statistically assigning a coupling or uncoupling probability to the determined amino acid residues and/or structural composition features,
c. Training a machine learning classifier using the coupling or uncoupling probabilities assigned to the one or more amino acid residues and/or one or more structural composition features of step b) in order to create a predictor for determining a probability of coupling or no coupling between a G-protein and a G-protein coupled receptor (GPCR),
d. Providing an amino acid sequence and/or 3D structural data of a query GPCR and applying the trained machine learning predictor of step c) in order to determine a probability that the query GPCR couples or uncouples to a predetermined G-protein and/or
e. Providing an amino acid sequence and/or 3D structural data of a query G-protein and applying the trained machine learning predictor of step c) in order to determine a probability that the query G-protein couples or uncouples to a predetermined GPCR.

4. The method according to claim 3, wherein the amino acid sequence data and/or 3D structural data of the G-protein in step a) is provided for at least part of one or more of G-protein sub-families G s, G i/o, G q/11, and G 12/13, preferably at least part of the α subunit of one or more of G-protein sub-families G s , G i/o, G q/11, and G 12/13 and/or
wherein the amino acid sequence data and/or 3D structural data of the GPCR is at least provided for part of the amino acid sequence data and/or 3D structural data of Class A GPCRs, preferably wherein the part of the Class A GPCRs comprises or consists of at least part of the amino acid sequence data and/or 3D structural data of
i. one or more of the seven transmembrane bundle (7TM) features, such as transmembrane bundle 1 (TM1), transmembrane bundle 2 (TM2), transmembrane bundle 3 (TM3), transmembrane bundle 4 (TM4), transmembrane bundle 5 (TM5), transmembrane bundle 6 (TM6), and transmembrane bundle 7 (TM7), more preferably TM3, TM5, and TM6, and/or
ii. one or more of extra 7TM features, such as N-terminal, one or more extracellular loops (ECL), one or more intracellular loops (ICL), and/or C-terminal region, more preferably intracellular loop 3 (ICL3), and C-terminal region.

5. The method according to claim 3, wherein the amino acid sequence data and/or three dimensional (3D) structural data for of step a) comprises i) at least for one given G-protein data set a set of data of two or more respective coupling GPCRs and/or two or more respective uncoupling GPCRs and/or ii) at least for one given GPCR data set a set of data of two or more respective coupling G-proteins and/or two or more respective uncoupling G-proteins.

6. The method according to claim 3, wherein the statistical alignment and assignment of coupling or uncoupling probability according to step b) uses a Hidden Markov Model (HMM) profile.

7. The method according to claim 3, wherein the one or more structural composition features of step b) comprise or consist of length and/or aliphatic, aromatic, hydrophilic, and/or sulfuric amino acid composition and/or electrostatic charge distribution of sequences determined to be statistically significantly associated with the coupled G-protein/GPCR complex or with the uncoupled G-protein/GPCR complex, wherein the one or more structural composition features are preferably selected from the group consisting of TM3, TM5, TM6, ICL3, and C-terminal region.

8. The method according to claim 3, wherein the training step c) using the machine learning predictor uses a logistic regression classifier.

9. The method according to claim 3, wherein the training step c) comprises a step of comparing the alignment and probability of coupling or uncoupling and assigning weight for coupling or uncoupling to the respective significant coupling and/or uncoupling amino acid residues and/or structural composition features.

10. The method according to claim 4, wherein the G-protein coupled receptor (GPCR) with the predetermined G-protein coupling profile is a Designer Receptor Exclusively Activated by Designer Drugs (DREADD).

11. A computer-implemented method for designing a G-protein coupled receptor (GPCR) with a predetermined G-protein coupling profile, wherein the method comprises or consists of the following steps:
a. Providing amino acid sequence data and/or three dimensional (3D) structural data of:
i. one or more G-proteins and one or more GPCRs known to couple as a G-protein/GPCR complex and
ii. one or more G-proteins and one or more GPCRs known to not couple as a G-protein/GPCR complex,
b. Statistically aligning the amino acid sequence and/or the 3D structural data of the G-protein with the GPCR of the respective coupled or uncoupled G-protein/GPCR complex provided in step a) in order to determine one or more amino acid residues and/or one or more structural composition features found to be statistically significantly associated with a coupled G-protein/GPCR complex or with an uncoupled G-protein/GPCR complex, and statistically assigning a coupling or uncoupling probability to the determined amino acid residues and/or structural composition features,
c. Training a machine learning classifier using the coupling or uncoupling probabilities assigned to the one or more amino acid residues and/or one or more structural composition features of step b) in order to create a predictor for determining a probability of coupling or no coupling between a G-protein and a G-protein coupled receptor (GPCR),
d. Providing an amino acid sequence and/or 3D structural data of a query GPCR and applying the trained machine learning predictor of step c) in order to determine a probability that the query GPCR couples or uncouples to a predetermined G-protein and designing a GPCR with a predetermined G-protein coupling profile by amending the amino acid sequence and/or the 3D structural data of the query GPCR in order to optimize the probability that the GPCR couples to the predetermined G-protein and optionally to optimize the probability to not couple to other G-proteins.

12. The method according to claim 11, wherein the amino acid sequence data and/or 3D structural data of the G-protein in step a) is provided for at least part of one or more of G-protein sub-families G s, G i/o, G q/11, and G 12/13, preferably at least part of the α subunit of one or more of G-protein sub-families G s , G i/o, G q/11, and G 12/13 and/or
wherein the amino acid sequence data and/or 3D structural data of the GPCR is at least provided for part of the amino acid sequence data and/or 3D structural data of Class A GPCRs, preferably wherein the part of the Class A GPCRs comprises or consists of at least part of the amino acid sequence data and/or 3D structural data of
i. one or more of the seven transmembrane bundle (7TM) features, such as transmembrane bundle 1 (TM1), transmembrane bundle 2 (TM2), transmembrane bundle 3 (TM3), transmembrane bundle 4 (TM4), transmembrane bundle 5 (TM5), transmembrane bundle 6 (TM6), and transmembrane bundle 7 (TM7), more preferably TM3, TM5, and TM6, and/or
ii. one or more of extra 7TM features, such as N-terminal, one or more extracellular loops (ECL), one or more intracellular loops (ICL), and/or C-terminal region, more preferably intracellular loop 3 (ICL3), and C-terminal region.

13. The method according to claim 11, wherein the amino acid sequence data and/or three dimensional (3D) structural data for of step a) comprises i) at least for one given G-protein data set a set of data of two or more respective coupling GPCRs and/or two or more respective uncoupling GPCRs and/or ii) at least for one given GPCR data set a set of data of two or more respective coupling G-proteins and/or two or more respective uncoupling G-proteins.

14. The method according to claim 11, wherein the statistical alignment and assignment of coupling or uncoupling probability according to step b) uses a Hidden Markov Model (HMM) profile.

15. The method according to claim 11, wherein the one or more structural composition features of step b) comprise or consist of length and/or aliphatic, aromatic, hydrophilic, and/or sulfuric amino acid composition and/or electrostatic charge distribution of sequences determined to be statistically significantly associated with the coupled G-protein/GPCR complex or with the uncoupled G-protein/GPCR complex, wherein the one or more structural composition features are preferably selected from the group consisting of TM3, TM5, TM6, ICL3, and C-terminal region.

16. The method according to claim 11, wherein the training step c) using the machine learning predictor uses a logistic regression classifier.

17. The method according to claim 11, wherein the training step c) comprises a step of comparing the alignment and probability of coupling or uncoupling and assigning weight for coupling or uncoupling to the respective significant coupling and/or uncoupling amino acid residues and/or structural composition features.

18. A computational data processing system comprising a data processing system having one or more processors coupled to a memory and inputting and outputting devices, wherein the data processing system is configured to:
a. determine a probability of coupling or no coupling between a G-protein and a G-protein coupled receptor (GPCR) according to the method steps of claim 1, or
b. design a G-protein coupled receptor (GPCR) with a predetermined G-protein coupling profile.

19. The method according to claim 18, alone or together with one or more further data sets relating to the same or other GPCR signaling pathways selected from the group consisting of genomic sequencing, transcriptomics, proteomics, and/or metabolomics in quantification of GPCR downstream signaling in normal and/or pathological conditions.


SEQUENCE LISTING (114 sequences)

SEQ ID NO: 1 - protein, 502 aa, Homo sapiens
  REGION 1..502: note = DREADD M3D
MDYKDDDDKGSTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDCVASNASVMNLLVISFDRYFSITRPLTYRAKRTTKRAGVMIGLAWVISFVLWAPAILFWQYFVGKRTVPPGECFIQFLSEPTITFGTAIAGFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQT

SEQ ID NO: 2 - protein, 388 aa, Homo sapiens
  REGION 1..388: note = M3D-GPR183/ICL3
MDYKDDDDKGSTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDCVASNASVMNLLVISFDRYFSITRPLTYRAKRTTKRAGVMIGLAWVISFVLWAPAILFWQYFVGKRTVPPGECFIQFLSEPTITFGTAIAGFYMPVTIMTILYWRIYKETERTAKQNPLTEKSGVEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 3 - protein, 1150 aa, Homo sapiens
  REGION 1..1150: note = M3D-GPR132/ICL3
METASPTYRLYSASPASPASPASPLYSGLYSERTHRLEUHISASNASNSERTHRTHRSERPROLEUPHEPROASNILESERSERSERTRPILEHISSERPROSERASPALAGLYLEUPROPROGLYTHRVALTHRHISPHEGLYSERTYRASNVALSERARGALAALAGLYASNPHESERSERPROASPGLYTHRTHRASPASPPROLEUGLYGLYHISTHRVALTRPGLNVALVALPHEILEALAPHELEUTHRGLYILELEUALALEUVALTHRILEILEGLYASNRILELEUVALILEVALSERPHELYSVALASNLYSGLNLEULYSTHRVALASNASNTYRPHELEULEUSERLEUALACYSALAASPLEUILEILEGLYVALILESERMETASNLEUPHETHRTHRTYRILEILEMETASNARGTRPALALEUGLYASNLEUALACYSASPLEUTRPLEUALAILEASPCYSVALALASERASNALASERVALMETASNLEULEUVALILESERPHEASPARGTYRPHESERILETHRARGPROLEUTHRTYRARGALALYSARGTHRTHRLYSARGALAGLYVALMETILEGLYLEUALATRPVALILESERPHEVALLEUTRPALAPROALAILELEUPHETRPGLNTYRPHEVALGLYLYSARGTHRVALPROPROGLYGLUCYSPHEILEGLNPHELEUSERGLUPROTHRILETHRPHEGLYTHRALAILEALAGLYPHETYRMETPROVALTHRILEMETTHRILELEUTYRTRPARGILETYRLYSGLUTHRGLUGLNSERMETGLYLEUSERALAALAGLNGLULYSLYSALAALAGLNTHRLEUSERALAILELEULEUALAPHEILEILETHRTRPTHRPROTYRASNILEMETVALLEUVALASNTHRPHECYSASPSERCYSILEPROLYSTHRPHETRPASNLEUGLYTYRTRPLEUCYSTYRILEASNSERTHRVALASNPROVALCYSTYRALALEUCYSASNLYSTHRPHEARGTHRTHRPHELYSMETLEULEULEUCYSGLNCYSASPLYSLYSLYSARGARGLYSGLNGLNTYRGLNGLNARGGLNSERVALILEPHEHISLYSARGALAPROGLUGLNALALEU

SEQ ID NO: 4 - protein, 388 aa, Homo sapiens
  REGION 1..388: note = M3D-GPR183/ICL3 (1.57V)
MDYKDDDDKGSTLHNNSTTSPLFPNISSSWIHSPSDAGLPPGTVTHFGSYNVSRAAGNFSSPDGTTDDPLGGHTVWQVVFIAFLTGILALVTIIGNILVIVSVKVNKQLKTVNNYFLLSLACADLIIGVISMNLFTTYIIMNRWALGNLACDLWLAIDCVASNASVMNLLVISFDRYFSITRPLTYRAKRTTKRAGVMIGLAWVISFVLWAPAILFWQYFVGKRTVPPGECFIQFLSEPTITFGTAIAGFYMPVTIMTILYWRIYKETERTAKQNPLTEKSGVEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 5 - protein, 582 aa, Homo sapiens
  REGION 1..582: note = G\'ce\'b1s-Lg (GNAS-LgBiT)
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 6 - protein, 542 aa, Homo sapiens
  REGION 1..542: note = G\'ce\'b1i1-Lg (GNAI1-LgBiT)
MGCTLSAEDKAAVERSKMIDRNLREDGEKAAREVKLLLLGAGESGKSTIVKQMKIIHEAGYSEEECKQYKAVVYSNTIQSIIAIIRAMGRLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF

SEQ ID NO: 7 - protein, 543 aa, Homo sapiens
  REGION 1..543: note = G\'ce\'b1i2-Lg (GNAI2-LgBiT)
MGCTVSAEDKAAAERSKMIDKNLREDGEKAAREVKLLLLGAGESGKSTIVKQMKIIHEDGYSEEECRQYRAVVYSNTIQSIMAIVKAMGNLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGLF

SEQ ID NO: 8 - protein, 542 aa, Homo sapiens
  REGION 1..542: note = G\'ce\'b1i3-Lg (GNAI3-LgBiT)
MGCTLSAEDKAAVERSKMIDRNLREDGEKAAKEVKLLLLGAGESGKSTIVKQMKIIHEDGYSEDECKQYKVVVYSNTIQSIIAIIRAMGRLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGKIDFGEAARADDARQLFVLAGSAEEGVMTPELAGVIKRLWRDGGVQACFSRSREYQLNDSASYYLNDLDRISQSNYIPTQQDVLRTRVKTTGIVETHFTFKDLYFKMFDVGGQRSERKKWIHCFEGVTAIIFCVALSDYDLVLAEDEEMNRMHESMKLFDSICNNKWFTETSIILFLNKKDLFEEKIKRSPLTICYPEYTGSNTYEEAAAYIQCQFEDLNRRKDTKEIYTHFTCATDTKNVQFVFDAVTDVIIKNNLKECGLY

SEQ ID NO: 9 - protein, 542 aa, Homo sapiens
  REGION 1..542: note = G\'ce\'b1o-Lg (GNAO1-LgBiT)
MGCTLSAEERAALERSKAIEKNLKEDGISAAKDVKLLLLGAGESGKSTIVKQMKIIHEDGFSGEDVKQYKPVVYSNTIQSLAAIVRAMDTLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGGIEYGDKERKADAKMVCDVVSRMEDTEPFSAELLSAMMRLWGDSGIQECFNRSREYQLNDSAKYYLDSLDRIGAADYQPTEQDILRTRVKTTGIVETHFTFKNLHFRLFDVGGQRSERKKWIHCFEDVTAIIFCVALSGYDQVLHEDETTNRMHESLMLFDSICNNKFFIDTSIILFLNKKDLFGEKIKKSPLTICFPEYTGPNTYEDAAAYIQAQFESKNRSPNKEIYCHMTCATDTNNIQVVFDAVTDIIIANNLRGCGLY

SEQ ID NO: 10 - protein, 547 aa, Homo sapiens
  REGION 1..547: note = G\'ce\'b1q-Lg (GNAQ-LgBiT)
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 11 - protein, 547 aa, Homo sapiens
  REGION 1..547: note = G\'ce\'b111-Lg (GNA11-LgBiT)
MTLESMMACCLSDEVKESKRINAEIEKQLRRDKRDARRELKLLLLGTGESGKSTFIKQMRIIHGAGYSEEDKRGFTKLVYQNIFTAMQAMIRAMETLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGKILYKYEQNKANALLIREVDVEKVTTFEHQYVSAIKTLWEDPGIQECYDRRREYQLSDSAKYYLTDVDRIATLGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILYSHLVDYFPEFDGPQRDAQAAREFILKMFVDLNPDSDKIIYSHFTCATDTENIRFVFAAVKDTILQLNLKEYNLV

SEQ ID NO: 12 - protein, 543 aa, Homo sapiens
  REGION 1..543: note = G\'ce\'b114-Lg (GNA14-LgBiT)
MAGCCCLSAEEKESQRISAEIERQLRRDKKDARRELKLLLLGTGESGKSTFIKQMRIIHGSGYSDEDRKGFTKLVYQNIFTAMQAMIRAMDTLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGRIQYVCEQNKENAQIIREVEVDKVSMLSREQVEAIKQLWQDPGIQECYDRRREYQLSDSAKYYLTDIDRIATPSFVPTQQDVLRVRVPTTGIIEYPFDLENIIFRMVDVGGQRSERRKWIHCFESVTSIIFLVALSEYDQVLAECDNENRMEESKALFKTIITYPWFLNSSVILFLNKKDLLEEKIMYSHLISYFPEYTGPKQDVRAARDFILKLYQDQNPDKEKVIYSHFTCATDTDNIRFVFAAVKDTILQLNLREFNLV

SEQ ID NO: 13 - protein, 562 aa, Homo sapiens
  REGION 1..562: note = G\'ce\'b116-Lg (GNA15-LgBiT)
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGQIPFSRPESKHHASLVMSQDPYKVTTFEKRYAAAMQWLWRDAGIRAYYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 14 - protein, 569 aa, Homo sapiens
  REGION 1..569: note = G\'ce\'b112-Lg (GNA12-LgBiT)
MSGVVRTLSRCLLPAEAGGARERRAGSGARDAEREARRRSRDIDALLARERRAVRRLVKILLLGAGESGKSTFLKQMRIIHGREFDQKALLEFRDTIFDNILKGSRVLVDARDKLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGGIPWQYSENEKHGMFLMAFENKAGLPVEPATFQLYVPALSALWRDSGIREAFSRRSEFQLGESVKYFLDNLDRIGQLNYFPSKQDILLARKATKGIVEHDFVIKKIPFKMVDVGGQRSQRQKWFQCFDGITSILFMVSSSEYDQVLMEDRRTNRLVESMNIFETIVNNKLFFNVSIILFLNKMDLLVEKVKTVSIKKHFPDFRGDPHRLEDVQRYLVQCFDRKRRNRSKPLFHHFTTAIDTENVRFVFHAVKDTILQENLKDIMLQ

SEQ ID NO: 15 - protein, 565 aa, Homo sapiens
  REGION 1..565: note = G\'ce\'b113-Lg (GNA13-LgBiT)
MADFLPSRSVLSVCFPGCLLTSGEAEQQRKSKEIDKCLSREKTYVKRLVKILLLGAGESGKSTFLKQMRIIHGQDFDQRAREEFRPTIYSNVIKGMRVLVDAREKLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGHIPWGDNSNQQHGDKMMSFDTRAPMAAQGMVETRVFLQYLPAIRALWADSGIQNAYDRRREFQLGESVKYFLDNLDKLGEPDYIPSQQDILLARRPTKGIHEYDFEIKNVPFKMVDVGGQRSERKRWFECFDSVTSILFLVSSSEFDQVLMEDRLTNRLTESLNIFETIVNNRVFSNVSIILFLNKTDLLEEKVQIVSIKDYFLEFEGDPHCLRDVQKFLVECFRNKRRDQQQKPLYHHFTTAINTENIRLVFRDVKDTILHDNLKQLMLQ

SEQ ID NO: 16 - protein, 366 aa, Homo sapiens
  REGION 1..366: note = Sm-G\'ce\'b21 (SmBiT-GNB1)
MVTGYRLFEEILGGSGGGGSGGSSSGGSELDQLRQEAEQLKNQIRDARKACADATLSQITNNIDPVGRIQMRTRRTLRGHLAKIYAMHWGTDSRLLVSASQDGKLIIWDSYTTNKVHAIPLRSSWVMTCAYAPSGNYVACGGLDNICSIYNLKTREGNVRVSRELAGHTGYLSCCRFLDDNQIVTSSGDTTCALWDIETGQQTTTFTGHTGDVMSLSLAPDTRLFVSGACDASAKLWDVREGMCRQTFTGHESDINAICFFPNGNAFATGSDDATCRLFDLRADQELMTYSHDNIICGITSVSFSKSGRLLLAGYDDFNCNVWDALKADRAGVLAGHDNRVSCLGVTDDGMAVATGSWDSFLKIWN

SEQ ID NO: 17 - protein, 366 aa, Homo sapiens
  REGION 1..366: note = Sm-G\'ce\'b22 (SmBiT-GNB2)
MVTGYRLFEEILGGSGGGGSGGSSSGGSELEQLRQEAEQLRNQIRDARKACGDSTLTQITAGLDPVGRIQMRTRRTLRGHLAKIYAMHWGTDSRLLVSASQDGKLIIWDSYTTNKVHAIPLRSSWVMTCAYAPSGNFVACGGLDNICSIYSLKTREGNVRVSRELPGHTGYLSCCRFLDDNQIITSSGDTTCALWDIETGQQTVGFAGHSGDVMSLSLAPDGRTFVSGACDASIKLWDVRDSMCRQTFIGHESDINAVAFFPNGYAFTTGSDDATCRLFDLRADQELLMYSHDNIICGITSVAFSRSGRLLLAGYDDFNCNIWDAMKGDRAGVLAGHDNRVSCLGVTDDGMAVATGSWDSFLKIWN

SEQ ID NO: 18 - protein, 366 aa, Homo sapiens
  REGION 1..366: note = Sm-G\'ce\'b23 (SmBiT-GNB3)
MVTGYRLFEEILGGSGGGGSGGSSSGGGEMEQLRQEAEQLKKQIADARKACADVTLAELVSGLEVVGRVQMRTRRTLRGHLAKIYAMHWATDSKLLVSASQDGKLIVWDSYTTNKVHAIPLRSSWVMTCAYAPSGNFVACGGLDNMCSIYNLKSREGNVKVSRELSAHTGYLSCCRFLDDNNIVTSSGDTTCALWDIETGQQKTVFVGHTGDCMSLAVSPDFNLFISGACDASAKLWDVREGTCRQTFTGHESDINAICFFPNGEAICTGSDDASCRLFDLRADQELICFSHESIICGITSVAFSLSGRLLFAGYDDFNCNVWDSMKSERVGILSGHDNRVSCLGVTADGMAVATGSWDSFLKIWN

SEQ ID NO: 19 - protein, 366 aa, Homo sapiens
  REGION 1..366: note = Sm-G\'ce\'b24 (SmBiT-GNB4)
MVTGYRLFEEILGGSGGGGSGGSSSGGSELEQLRQEAEQLRNQIQDARKACNDATLVQITSNMDSVGRIQMRTRRTLRGHLAKIYAMHWGYDSRLLVSASQDGKLIIWDSYTTNKMHAIPLRSSWVMTCAYAPSGNYVACGGLDNICSIYNLKTREGNVRVSRELPGHTGYLSCCRFLDDSQIVTSSGDTTCALWDIETAQQTTTFTGHSGDVMSLSLSPDMRTFVSGACDASSKLWDIRDGMCRQSFTGHVSDINAVSFFPNGYAFATGSDDATCRLFDLRADQELLLYSHDNIICGITSVAFSKSGRLLLAGYDDFNCNVWDTLKGDRAGVLAGHDNRVSCLGVTDDGMAVATGSWDSFLRIWN

SEQ ID NO: 20 - protein, 421 aa, Homo sapiens
  REGION 1..421: note = Sm-G\'ce\'b25 (SmBiT-GNB5)
MVTGYRLFEEILGGSGGGGSGGSSSGGCDQTFLVNVFGSCDKCFKQRALRPVFKKSQQLSYCSTCAEIMATEGLHENETLASLKSEAESLKGKLEEERAKLHDVELHQVAERVEALGQFVMKTRRTLKGHGNKVLCMDWCKDKRRIVSSSQDGKVIVWDSFTTNKEHAVTMPCTWVMACAYAPSGCAIACGGLDNKCSVYPLTFDKNENMAAKKKSVAMHTNYLSACSFTNSDMQILTASGDGTCALWDVESGQLLQSFHGHGADVLCLDLAPSETGNTFVSGGCDKKAMVWDMRSGQCVQAFETHESDINSVRYYPSGDAFASGSDDATCRLYDLRADREVAIYSKESIIFGASSVDFSLSGRLLFAGYNDYTINVWDVLKGSRVSILFGHENRVSTLRVSPDGTAFCSGSWDHTLRVWA

SEQ ID NO: 21 - protein, 97 aa, Homo sapiens
  REGION 1..97: note = Sm-G\'ce\'b32 (SmBiT-GNG2)
MVTGYRLFEEILGGSGGGGSGGSSSGGASNNTASIAQARKLVEQLKMEANIDRIKVSKAAADLMAYCEAHAKEDPLLTPVPASENPFREKKFFCAIL

SEQ ID NO: 22 - protein, 101 aa, Homo sapiens
  REGION 1..101: note = Sm-G\'ce\'b33 (SmBiT-GNG3)
MVTGYRLFEEILGGSGGGGSGGSSSGGKGETPVNSTMSIGQARKMVEQLKIEASLCRIKVSKAAADLMTYCDAHACEDPLITPVPTSENPFREKKFFCALL

SEQ ID NO: 23 - protein, 101 aa, Homo sapiens
  REGION 1..101: note = Sm-G\'ce\'b34 (SmBiT-GNG4)
MVTGYRLFEEILGGSGGGGSGGSSSGGKEGMSNNSTTSISQARKAVEQLKMEACMDRVKVSQAAADLLAYCEAHVREDPLIIPVPASENPFREKKFFCTIL

SEQ ID NO: 24 - protein, 94 aa, Homo sapiens
  REGION 1..94: note = Sm-G\'ce\'b35 (SmBiT-GNG5)
MVTGYRLFEEILGGSGGGGSGGSSSGGSGSSSVAAMKKVVQQLRLEAGLNRVKVSQAAADLKQFCLQNAQHDPLLTGVSSSTNPFRPQKVCSFL

SEQ ID NO: 25 - protein, 94 aa, Homo sapiens
  REGION 1..94: note = Sm-G\'ce\'b37 (SmBiT-GNG7)
MVTGYRLFEEILGGSGGGGSGGSSSGGSATNNIAQARKLVEQLRIEAGIERIKVSKAASDLMSYCEQHARNDPLLVGVPASENPFKDKKPCIIL

SEQ ID NO: 26 - protein, 96 aa, Homo sapiens
  REGION 1..96: note = Sm-G\'ce\'b38 (SmBiT-GNG8)
MVTGYRLFEEILGGSGGGGSGGSSSGGSNNMAKIAEARKTVEQLKLEVNIDRMKVSQAAAELLAFCETHAKDDPLVTPVPAAENPFRDKRLFCVLL

SEQ ID NO: 27 - protein, 94 aa, Homo sapiens
  REGION 1..94: note = Sm-G\'ce\'b310 (SmBiT-GNG10)
MVTGYRLFEEILGGSGGGGSGGSSSGGSSGASASALQRLVEQLKLEAGVERIKVSQAAAELQQYCMQNACKDALLVGVPAGSNPFREPRSCALL

SEQ ID NO: 28 - protein, 99 aa, Homo sapiens
  REGION 1..99: note = Sm-G\'ce\'b311 (SmBiT-GNG11)
MVTGYRLFEEILGGSGGGGSGGSSSGGPALHIEDLPEKEKLKMEVEQLRKEVKLQRQQVSKCSEEIKNYIEERSGEDPLVKGIPEDKNPFKEKGSCVIS

SEQ ID NO: 29 - protein, 98 aa, Homo sapiens
  REGION 1..98: note = Sm-G\'ce\'b312 (SmBiT-GNG12)
MVTGYRLFEEILGGSGGGGSGGSSSGGSSKTASTNNIAQARRTVQQLRLEASIERIKVSKASADLMSYCEEHARSDPLLIGIPTSENPFKDKKTCIIL

SEQ ID NO: 30 - protein, 93 aa, Homo sapiens
  REGION 1..93: note = Sm-G\'ce\'b313 (SmBiT-GNG13)
MVTGYRLFEEILGGSGGGGSGGSSSGGEEWDVPQMKKEVESLKYQLAFQREMASKTIPELLKWIEDGIPKDPFLNPDLMKNNPWVEKGKCTIL

SEQ ID NO: 31 - protein, 100 aa, Homo sapiens
  REGION 1..100: note = Sm-G\'ce\'b3t1 (SmBiT-GNGT1)
MVTGYRLFEEILGGSGGGGSGGSSSGGPVINIEDLTEKDKLKMEVDQLKKEVTLERMLVSKCCEEVRDYVEERSGEDPLVKGIPEDKNPFKELKGGCVIS

SEQ ID NO: 32 - protein, 95 aa, Homo sapiens
  REGION 1..95: note = Sm-G\'ce\'b3 (SmBiT-GNGT2)
MVTGYRLFEEILGGSGGGGSGGSSSGGAQDLSEKDLLKMEVEQLKKEVKNTRIPISKAGKEIKEYVEAQAGNDPFLKGIPEDKNPFKEKGGCLIS

SEQ ID NO: 33 - protein, 435 aa, Homo sapiens
  REGION 1..435: note = G\'ce\'b1s-Sm (GNAS-SmBiT)
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLGGSGGGGSGGSSSGGVTGYRLFEEILGGSGGGGSGGSSSGGVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 34 - protein, 568 aa, Homo sapiens
  REGION 1..568: note = G\'ce\'b1s-Lg (\'ce\'b1B-\'ce\'b1C) (GNAS-LgBiT-\'ce\'b1B-\'ce\'b1C)
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 35 - protein, 421 aa, Homo sapiens
  REGION 1..421: note = G\'ce\'b1s-Sm (\'ce\'b1B-\'ce\'b1C) (GNAS-SmBiT-\'ce\'b1B-\'ce\'b1C)
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFGGSGGGGSGGSSSGGVTGYRLFEEILGGSGGGGSGGSSSGGDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 36 - protein, 582 aa, Homo sapiens
  REGION 1..582: note = G\'ce\'b1s-Lg (G2A/C3S) (GNAS-LgBiT-G2A-C3S)
MASLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 37 - protein, 547 aa, Homo sapiens
  REGION 1..547: note = G\'ce\'b1q-Lg (R183C) (GNAQ-LgBiT-R183C)
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGKIPYKYEHNKAHAQLVREVDVEKVSAFENPYVDAIKSLWNDPGIQECYDRRREYQLSDSTKYYLNDLDRVADPAYLPTQQDVLRVCVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 38 - protein, 547 aa, Homo sapiens
  REGION 1..547: note = G\'ce\'b1q-Lg (\'ce\'b1B-\'ce\'b1C) (GNAQ-LgBiT-\'ce\'b1B-\'ce\'b1C)
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 39 - protein, 547 aa, Homo sapiens
  REGION 1..547: note = G\'ce\'b111-Lg (\'ce\'b1B-\'ce\'b1C) (GNA11-LgBiT-\'ce\'b1B-\'ce\'b1C)
MTLESMMACCLSDEVKESKRINAEIEKQLRRDKRDARRELKLLLLGTGESGKSTFIKQMRIIHGAGYSEEDKRGFTKLVYQNIFTAMQAMIRAMETLKILYKYEQNKANALLIREVDVEKVTTGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGFEHQYVSAIKTLWEDPGIQECYDRRREYQLSDSAKYYLTDVDRIATLGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILYSHLVDYFPEFDGPQRDAQAAREFILKMFVDLNPDSDKIIYSHFTCATDTENIRFVFAAVKDTILQLNLKEYNLV

SEQ ID NO: 40 - protein, 543 aa, Homo sapiens
  REGION 1..543: note = G\'ce\'b114-Lg (\'ce\'b1B-\'ce\'b1C) (GNA14-LgBiT-\'ce\'b1B-\'ce\'b1C)
MAGCCCLSAEEKESQRISAEIERQLRRDKKDARRELKLLLLGTGESGKSTFIKQMRIIHGSGYSDEDRKGFTKLVYQNIFTAMQAMIRAMDTLRIQYVCEQNKENAQIIREVEVDKVSMGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGLSREQVEAIKQLWQDPGIQECYDRRREYQLSDSAKYYLTDIDRIATPSFVPTQQDVLRVRVPTTGIIEYPFDLENIIFRMVDVGGQRSERRKWIHCFESVTSIIFLVALSEYDQVLAECDNENRMEESKALFKTIITYPWFLNSSVILFLNKKDLLEEKIMYSHLISYFPEYTGPKQDVRAARDFILKLYQDQNPDKEKVIYSHFTCATDTDNIRFVFAAVKDTILQLNLREFNLV

SEQ ID NO: 41 - protein, 562 aa, Homo sapiens
  REGION 1..562: note = G\'ce\'b116-Lg (\'ce\'b1B-\'ce\'b1C) (GNA15-LgBiT-\'ce\'b1B-\'ce\'b1C)
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTGGSGGGGSGGSSSGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGFEKRYAAAMQWLWRDAGIRAYYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 42 - protein, 513 aa, Homo sapiens
  REGION 1..513: note = Lg-G\'ce\'b21 (LgBiT-GNB1)
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGSELDQLRQEAEQLKNQIRDARKACADATLSQITNNIDPVGRIQMRTRRTLRGHLAKIYAMHWGTDSRLLVSASQDGKLIIWDSYTTNKVHAIPLRSSWVMTCAYAPSGNYVACGGLDNICSIYNLKTREGNVRVSRELAGHTGYLSCCRFLDDNQIVTSSGDTTCALWDIETGQQTTTFTGHTGDVMSLSLAPDTRLFVSGACDASAKLWDVREGMCRQTFTGHESDINAICFFPNGNAFATGSDDATCRLFDLRADQELMTYSHDNIICGITSVSFSKSGRLLLAGYDDFNCNVWDALKADRAGVLAGHDNRVSCLGVTDDGMAVATGSWDSFLKIWN

SEQ ID NO: 43 - protein, 244 aa, Homo sapiens
  REGION 1..244: note = Lg-G\'ce\'b32 (LgBiT-GNG2)
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGASNNTASIAQARKLVEQLKMEANIDRIKVSKAAADLMAYCEAHAKEDPLLTPVPASENPFREKKFFCAIL

SEQ ID NO: 44 - protein, 97 aa, Homo sapiens
  REGION 1..97: note = Sm-G\'ce\'b32 (C68S) (SmBiT-GNG2-C68S)
MVTGYRLFEEILGGSGGGGSGGSSSGGASNNTASIAQARKLVEQLKMEANIDRIKVSKAAADLMAYCEAHAKEDPLLTPVPASENPFREKKFFSAIL

SEQ ID NO: 45 - protein, 1242 aa, Homo sapiens
  REGION 1..1242: note = Sm-PLC\'ce\'b21 (SmBiT-PLCB1)
MVTGYRLFEEILGGSGGGGSGGSSSGGAGAQPGVHALQLKPVCVSDSLKKGTKFVKWDDDSTIVTPIILRTDPQGFFFYWTDQNKETELLDLSLVKDARCGRHAKAPKDPKLRELLDVGNIGRLEQRMITVVYGPDLVNISHLNLVAFQEEVAKEWTNEVFSLATNLLAQNMSRDAFLEKAYTKLKLQVTPEGRIPLKNIYRLFSADRKRVETALEACSLPSSRNDSIPQEDFTPEVYRVFLNNLCPRPEIDNIFSEFGAKSKPYLTVDQMMDFINLKQRDPRLNEILYPPLKQEQVQVLIEKYEPNNSLARKGQISVDGFMRYLSGEENGVVSPEKLDLNEDMSQPLSHYFINSSHNTYLTAGQLAGNSSVEMYRQVLLSGCRCVELDCWKGRTAEEEPVITHGFTMTTEISFKEVIEAIAECAFKTSPFPILLSFENHVDSPKQQAKMAEYCRLIFGDALLMEPLEKYPLESGVPLPSPMDLMYKILVKNKKKSHKSSEGSGKKKLSEQASNTYSDSSSMFEPSSPGAGEADTESDDDDDDDDCKKSSMDEGTAGSEAMATEEMSNLVNYIQPVKFESFEISKKRNKSFEMSSFVETKGLEQLTKSPVEFVEYNKMQLSRIYPKGTRVDSSNYMPQLFWNAGCQMVALNFQTMDLAMQINMGMYEYNGKSGYRLKPEFMRRPDKHFDPFTEGIVDGIVANTLSVKIISGQFLSDKKVGTYVEVDMFGLPVDTRRKAFKTKTSQGNAVNPVWEEEPIVFKKVVLPTLACLRIAVYEEGGKFIGHRILPVQAIRPGYHYICLRNERNQPLTLPAVFVYIEVKDYVPDTYADVIEALSNPIRYVNLMEQRAKQLAALTLEDEEEVKKEADPGETPSEAPSEARTTPAENGVNHTTTLTPKPPSQALHSQPAPGSVKAPAKTEDLIQSVLTEVEAQTIEELKQQKSFVKLQKKHYKEMKDLVKRHHKKTTDLIKEHTTKYNEIQNDYLRRRAALEKSAKKDSKKKSEPSSPDHGSSTIEQDLAALDAEMTQKLIDLKDKQQQQLLNLRQEQYYSEKYQKREHIKLLIQKLTDVAEECQNNQLKKLKEICEKEKKELKKKMDKKRQEKITEAKSKDKSQMEEEKTEMIRSYIQEVVQYIKRLEEAQSKRQEKLVEKHKEIRQQILDEKPKLQVELEQEYQDKFKRLPLEILEFVQEAMKGKISEDSNHGSAPLSLSSDPGKVNHKTPSSEELGGDIPGKEFDTPL

SEQ ID NO: 46 - protein, 1207 aa, Homo sapiens
  REGION 1..1207: note = Sm-PLC\'ce\'b22 (SmBiT-PLCB2)
MVTGYRLFEEILGGSGGGGSGGSSSGGSLLNPVLLPPKVKAYLSQGERFIKWDDETTVASPVILRVDPKGYYLYWTYQSKEMEFLDITSIRDTRFGKFAKMPKSQKLRDVFNMDFPDNSFLLKTLTVVSGPDMVDLTFHNFVSYKENVGKAWAEDVLALVKHPLTANASRSTFLDKILVKLKMQLNSEGKIPVKNFFQMFPADRKRVEAALSACHLPKGKNDAINPEDFPEPVYKSFLMSLCPRPEIDEIFTSYHAKAKPYMTKEHLTKFINQKQRDSRLNSLLFPPARPDQVQGLIDKYEPSGINAQRGQLSPEGMVWFLCGPENSVLAQDKLLLHHDMTQPLNHYFINSSHNTYLTAGQFSGLSSAEMYRQVLLSGCRCVELDCWKGKPPDEEPIITHGFTMTTDIFFKEAIEAIAESAFKTSPYPIILSFENHVDSPRQQAKMAEYCRTIFGDMLLTEPLEKFPLKPGVPLPSPEDLRGKILIKNKKNQFSGPTSSSKDTGGEAEGSSPPSAPAVWAGEEGTELEEEEVEEEEEEESGNLDEEEIKKMQSDEGTAGLEVTAYEEMSSLVNYIQPTKFVSFEFSAQKNRSYVISSFTELKAYDLLSKASVQFVDYNKRQMSRIYPKGTRMDSSNYMPQMFWNAGCQMVALNFQTMDLPMQQNMAVFEFNGQSGYLLKHEFMRRPDKQFNPFSVDRIDVVVATTLSITVISGQFLSERSVRTYVEVELFGLPGDPKRRYRTKLSPSTNSINPVWKEEPFVFEKILMPELASLRVAVMEEGNKFLGHRIIPINALNSGYHHLCLHSESNMPLTMPALFIFLEMKDYIPGAWADLTVALANPIKFFSAHDTKSVKLKEAMGGLPEKPFPLASPVASQVNGALAPTSNGSPAARAGAREEAMKEAAEPRTASLEELRELKGVVKLQRRHEKELRELERRGARRWEELLQRGAAQLAELGPPGVGGVGACKLGPGKGSRKKRSLPREESAGAAPGEGPEGVDGRVRELKDRLELELLRQGEEQYECVLKRKEQHVAEQISKMMELAREKQAAELKALKETSENDTKEMKKKLETKRLERIQGMTKVTTDKMAQERLKREINNSHIQEVVQVIKQMTENLERHQEKLEEKQAACLEQIREMEKQFQKEALAEYEARMKGLEAEVKESVRACLRTCFPSEAKDKPERACECPPELCEQDPLIAKADAQESRL

SEQ ID NO: 47 - protein, 1260 aa, Homo sapiens
  REGION 1..1260: note = Sm-PLC\'ce\'b23 (SmBiT-PLCB3)
MVTGYRLFEEILGGSGGGGSGGSSSGGAGAQPGVHALQLEPPTVVETLRRGSKFIKWDEETSSRNLVTLRVDPNGFFLYWTGPNMEVDTLDISSIRDTRTGRYARLPKDPKIREVLGFGGPDARLEEKLMTVVSGPDPVNTVFLNFMAVQDDTAKVWSEELFKLAMNILAQNASRNTFLRKAYTKLKLQVNQDGRIPVKNILKMFSADKKRVETALESCGLKFNRSESIRPDEFSLEIFERFLNKLCLRPDIDKILLEIGAKGKPYLTLEQLMDFINQKQRDPRLNEVLYPPLRPSQARLLIEKYEPNQQFLERDQMSMEGFSRYLGGEENGILPLEALDLSTDMTQPLSAYFINSSHNTYLTAGQLAGTSSVEMYRQALLWGCRCVELDVWKGRPPEEEPFITHGFTMTTEVPLRDVLEAIAETAFKTSPYPVILSFENHVDSAKQQAKMAEYCRSIFGDALLIEPLDKYPLAPGVPLPSPQDLMGRILVKNKKRHRPSAGGPDSAGRKRPLEQSNSALSESSAATEPSSPQLGSPSSDSCPGLSNGEEVGLEKPSLEPQKSLGDEGLNRGPYVLGPADREDEEEDEEEEEQTDPKKPTTDEGTASSEVNATEEMSTLVNYIEPVKFKSFEAARKRNKCFEMSSFVETKAMEQLTKSPMEFVEYNKQQLSRIYPKGTRVDSSNYMPQLFWNVGCQLVALNFQTLDVAMQLNAGVFEYNGRSGYLLKPEFMRRPDKSFDPFTEVIVDGIVANALRVKVISGQFLSDRKVGIYVEVDMFGLPVDTRRKYRTRTSQGNSFNPVWDEEPFDFPKVVLPTLASLRIAAFEEGGKFVGHRILPVSAIRSGYHYVCLRNEANQPLCLPALLIYTEASDYIPDDHQDYAEALINPIKHVSLMDQRARQLAALIGESEAQAGQETCQDTQSQQLGSQPSSNPTPSPLDASPRRPPGPTTSPASTSLSSPGQRDDLIASILSEVAPTPLDELRGHKALVKLRSRQERDLRELRKKHQRKAVTLTRRLLDGLAQAQAEGRCRLRPGALGGAADVEDTKEGEDEAKRYQEFQNRQVQSLLELREAQVDAEAQRRLEHLRQALQRLREVVLDANTTQFKRLKEMNEREKKELQKILDRKRHNSISEAKMRDKHKKEAELTEINRRHITESVNSIRRLEEAQKQRHDRLVAGQQQVLQQLAEEEPKLLAQLAQECQEQRARLPQEIRRSLLGEMPEGLGDGPLVACASNGHAPGSSGHLSGADSESQEENTQL

SEQ ID NO: 48 - protein, 1220 aa, Homo sapiens
  REGION 1..1220: note = Sm-PLC\'ce\'b24 (SmBiT-PLCB4)
MVTGYRLFEEILGGSGGGGSGGSSSGGAKPYEFNWQKEVPSFLQEGAVFDRYEEESFVFEPNCLFKVDEFGFFLTWRSEGKEGQVLECSLINSIRSGAIPKDPKILAALEAVGKSENDLEGRIVCVCSGTDLVNISFTYMVAENPEVTKQWVEGLRSIIHNFRANNVSPMTCLKKHWMKLAFMTNTNGKIPVRSITRTFASGKTEKVIFQALKELGLPSGKNDEIEPTAFSYEKFYELTQKICPRTDIEDLFKKINGDKTDYLTVDQLVSFLNEHQRDPRLNEILFPFYDAKRAMQIIEMYEPDEDLKKKGLISSDGFCRYLMSDENAPVFLDRLELYQEMDHPLAHYFISSSHNTYLTGRQFGGKSSVEMYRQVLLAGCRCVELDCWDGKGEDQEPIITHGKAMCTDILFKDVIQAIKETAFVTSEYPVILSFENHCSKYQQYKMSKYCEDLFGDLLLKQALESHPLEPGRALPSPNDLKRKILIKNKRLKPEVEKKQLEALRSMMEAGESASPANILEDDNEEEIESADQEEEAHPEFKFGNELSADDLGHKEAVANSVKKGLVTVEDEQAWMASYKYVGATTNIHPYLSTMINYAQPVKFQGFHVAEERNIHYNMSSFNESVGLGYLKTHAIEFVNYNKRQMSRIYPKGGRVDSSNYMPQIFWNAGCQMVSLNYQTPDLAMQLNQGKFEYNGSCGYLLKPDFMRRPDRTFDPFSETPVDGVIAATCSVQVISGQFLSDKKIGTYVEVDMYGLPTDTIRKEFRTRMVMNNGLNPVYNEESFVFRKVILPDLAVLRIAVYDDNNKLIGQRILPLDGLQAGYRHISLRNEGNKPLSLPTIFCNIVLKTYVPDGFGDIVDALSDPKKFLSITEKRADQMRAMGIETSDIADVPSDTSKNDKKGKANTAKANVTPQSSSELRPTTTAALASGVEAKKGIELIPQVRIEDLKQMKAYLKHLKKQQKELNSLKKKHAKEHSTMQKLHCTQVDKIVAQYDKEKSTHEKILEKAMKKKGGSNCLEMKKETEIKIQTLTSDHKSKVKEIVAQHTKEWSEMINTHSAEEQEIRDLHLSQQCELLKKLLINAHEQQTQQLKLSHDRESKEMRAHQAKISMENSKAISQDKSIKNKAERERRVRELNSSNTKKFLEERKRLAMKQSKEMDQLKKVQLEHLEFLEKQNEQLLKSCHAVSQTQGEGDAADGEIGSRDGPQTSNSSMKLQNAN

SEQ ID NO: 49 - protein, 158 aa, Homo sapiens
  REGION 1..158: note = LgBiT luciferase fragment
VFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 50 - protein, 12 aa, Homo sapiens
MVTGYRLFEEIL

SEQ ID NO: 51 - protein, 15 aa, Homo sapiens
GGSGGGGSGGSSSGG

SEQ ID NO: 52 - protein, 8 aa, Homo sapiens
DYKDDDDK

SEQ ID NO: 53 - protein, 14 aa, Homo sapiens
  REGION 1..14: note = Flag epitope DREADD without linker GS / GSG
RTAKQNPLTEKSGV

SEQ ID NO: 54 - protein, 9 aa, Homo sapiens
QSMGLSAAQ

SEQ ID NO: 55 - protein, 367 aa, Homo sapiens
  REGION 1..367: note = Lg-RhoA (LgBiT-RHOA)
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGTAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 56 - protein, 127 aa, Homo sapiens
  REGION 1..127: note = Sm-PKN1 (SmBiT-PKN1-GBD)
MVTGYRLFEEILGGSGGGGSGGSSSGGESEPRSWSLLEQLGLAGADLAAPGVQQQLELERERLRREIRKELKLKEGAENLRRATTDLGRSLGPVELLLRGSSRRLDLLHQQLQELHAHVVLPDPAAT

SEQ ID NO: 57 - protein, 581 aa, Homo sapiens
  REGION 1..581: note = Lg-IP3R2-Sm (LgBiT-ITPR2-IBC-SmBiT)
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGSGGGGSGGSSSGGTKYSSYREDVLKGGDVVRLFHAEQEKFLTCDEYEKKQHIFLRTTLRQSATSATSSKALWEIEVVHHDPCRGGAGQWNSLFRFKHLATGNYLAAELNPDYRDAQNEGKNVRDGVPPTSKKKRQAGEKIMYTLVSVPHGNDIASLFELDATTLQRADCLVPRNSYVRLRHLCTNTWVTSTSIPIDTDEERPVMLKIGTCQTKEDKEAFAIVSVPLSEVRDLDFANDANKVLATTVKKLENGTITQNERRFVTKLLEDLIFFVADVPNNGQEVLDVVITKPNRERQKLMREQNILAQVFGILKAPFKEKAGEGSMLRLEDLGDQRYAPYKYMLRLCYRVLRHSQQDYRKNQEYIAKNFCVMQSQIGYDILAEDTITALLHNNRKGGSGGGGSGGSSSGGVTGYRLFEEIL

SEQ ID NO: 58 - protein, 352 aa, Homo sapiens
  REGION 1..352: note = G\'ce\'b1q (\'e2\u710 \'88\u8224 \'86C)
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLN

SEQ ID NO: 59 - protein, 359 aa, Homo sapiens
  REGION 1..359: note = G\'ce\'b1q/s
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLRQYELL

SEQ ID NO: 60 - protein, 359 aa, Homo sapiens
  REGION 1..359: note = G\'ce\'b1q/olf
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKQYELL

SEQ ID NO: 61 - protein, 359 aa, Homo sapiens
  REGION 1..359: note = G\'ce\'b1q/i1
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDCGLF

SEQ ID NO: 62 - protein, 359 aa, Homo sapiens
  REGION 1..359: note = G\'ce\'b1q/i3
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKECGLY

SEQ ID NO: 63 - protein, 359 aa, Homo sapiens
  REGION 1..359: note = G\'ce\'b1q/o
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLRGCGLY

SEQ ID NO: 64 - protein, 359 aa, Homo sapiens
  REGION 1..359: note = G\'ce\'b1q/z
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKYIGLC

SEQ ID NO: 65 - protein, 359 aa, Homo sapiens
  REGION 1..359: note = G\'ce\'b1q
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 66 - protein, 359 aa, Homo sapiens
  REGION 1..359: note = G\'ce\'b1q/14
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLREFNLV

SEQ ID NO: 67 - protein, 359 aa, Homo sapiens
  REGION 1..359: note = G\'ce\'b1q/16
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLDEINLL

SEQ ID NO: 68 - protein, 359 aa, Homo sapiens
  REGION 1..359: note = G\'ce\'b1q/12
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKDIMLQ

SEQ ID NO: 69 - protein, 359 aa, Homo sapiens
  REGION 1..359: note = G\'ce\'b1q/13
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKQLMLQ

SEQ ID NO: 70 - protein, 374 aa, Homo sapiens
  REGION 1..374: note = G\'ce\'b1s (\'e2\u710 \'88\u8224 \'86C)
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHL

SEQ ID NO: 71 - protein, 380 aa, Homo sapiens
  REGION 1..380: note = G\'ce\'b1s
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 72 - protein, 380 aa, Homo sapiens
  REGION 1..380: note = G\'ce\'b1s/i1
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLKDCGLF

SEQ ID NO: 73 - protein, 380 aa, Homo sapiens
  REGION 1..380: note = G\'ce\'b1s/i3
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLKECGLY

SEQ ID NO: 74 - protein, 380 aa, Homo sapiens
  REGION 1..380: note = G\'ce\'b1s/o
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRGCGLY

SEQ ID NO: 75 - protein, 380 aa, Homo sapiens
  REGION 1..380: note = G\'ce\'b1s/z
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLKYIGLC

SEQ ID NO: 76 - protein, 380 aa, Homo sapiens
  REGION 1..380: note = G\'ce\'b1s/q
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLKEYNLV

SEQ ID NO: 77 - protein, 380 aa, Homo sapiens
  REGION 1..380: note = G\'ce\'b1s/14
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLREFNLV

SEQ ID NO: 78 - protein, 380 aa, Homo sapiens
  REGION 1..380: note = G\'ce\'b1s/16
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLDEINLL

SEQ ID NO: 79 - protein, 380 aa, Homo sapiens
  REGION 1..380: note = G\'ce\'b1s/12
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLKDIMLQ

SEQ ID NO: 80 - protein, 380 aa, Homo sapiens
  REGION 1..380: note = G\'ce\'b1s/13
MGCLGNSKTEDQRNEEKAQREANKKIEKQLQKDKQVYRATHRLLLLGAGESGKSTIVKQMRILHVNGFNGDSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLKQLMLQ

SEQ ID NO: 81 - protein, 6 aa, Homo sapiens
  REGION 1..6: note = C-terminally substituted 6-amino acids of G\'ce\'b1q/s
RQYELL

SEQ ID NO: 82 - protein, 6 aa, Homo sapiens
  REGION 1..6: note = C-terminally substituted 6-amino acids of G\'ce\'b1q/olf, G\'ce\'b1s/olf
KQYELL

SEQ ID NO: 83 - protein, 6 aa, Homo sapiens
  REGION 1..6: note = C-terminally substituted 6-amino acids of G\'ce\'b1q/i1, G\'ce\'b1s/i1
KDCGLF

SEQ ID NO: 84 - protein, 6 aa, Homo sapiens
  REGION 1..6: note = C-terminally substituted 6-amino acids of G\'ce\'b1q/i3, G\'ce\'b1s/i3
KECGLY

SEQ ID NO: 85 - protein, 6 aa, Homo sapiens
  REGION 1..6: note = C-terminally substituted 6-amino acids of G\'ce\'b1q/o, G\'ce\'b1s/o
RGCGLY

SEQ ID NO: 86 - protein, 6 aa, Homo sapiens
  REGION 1..6: note = C-terminally substituted 6-amino acids of G\'ce\'b1q/z, G\'ce\'b1s/z
KYIGLC

SEQ ID NO: 87 - protein, 6 aa, Homo sapiens
  REGION 1..6: note = C-terminally substituted 6-amino acids of G\'ce\'b1q/14, G\'ce\'b1s/14
REFNLV

SEQ ID NO: 88 - protein, 6 aa, Homo sapiens
  REGION 1..6: note = C-terminally substituted 6-amino acids of G\'ce\'b1q/16, G\'ce\'b1s/16
DEINLL

SEQ ID NO: 89 - protein, 6 aa, Homo sapiens
  REGION 1..6: note = C-terminally substituted 6-amino acids of G\'ce\'b1q/12
KDIMLQ

SEQ ID NO: 90 - protein, 6 aa, Homo sapiens
  REGION 1..6: note = C-terminally substituted 6-amino acids of G\'ce\'b1q/13, G\'ce\'b1s/13
KQLMLQ

SEQ ID NO: 91 - protein, 6 aa, Homo sapiens
  REGION 1..6: note = C-terminally substituted 6-amino acids of G\'ce\'b1s/q
KEYNLV

SEQ ID NO: 92 - protein, 6 aa, Homo sapiens
  REGION 1..6: note = C-terminally substituted 6-amino acids of G\'ce\'b1s/12
KDIMLQ

SEQ ID NO: 93 - protein, 651 aa, Homo sapiens
  REGION 1..651: note = amino acid sequence of the AP-TGF\'ce\'b1.
MVPSAGQLALFALGIVLAACQALENSTSPLSADPPVAAAVVSRSGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYHDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV

SEQ ID NO: 94 - protein, 39 aa, Homo sapiens
  REGION 1..39: note = signal-sequence peptide and the cleaved fragment of human TGFA
MVPSAGQLALFALGIVLAACQALENSTSPLSADPPVAAA

SEQ ID NO: 95 - protein, 489 aa, Homo sapiens
  REGION 1..489: note = alkaline phosphatase sequence derived from human ALPP
GIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHP

SEQ ID NO: 96 - protein, 48 aa, Homo sapiens
  REGION 1..48: note = mature, ectodomain-shed fragment of human TGFA
GYHDCPDSHTQFCFHGTCRFLVQEDKPACVCHSGYVGARCEHADLLAV

SEQ ID NO: 97 - RNA, 25 nt, Homo sapiens
  misc_feature 1..25: note = GNAQHSS104236
  source 1..25: mol_type = mRNA
ggagagagtggcaagagtacgttta

SEQ ID NO: 98 - RNA, 25 nt, Homo sapiens
  misc_feature 1..25: note = GNAQHSS104237
  source 1..25: mol_type = mRNA
ccctttgacttacaaagtgtcattt

SEQ ID NO: 99 - RNA, 25 nt, Homo sapiens
  misc_feature 1..25: note = GNA11HSS178464
  source 1..25: mol_type = mRNA
ccggcatcatcgagtaccctttcga

SEQ ID NO: 100 - RNA, 25 nt, Homo sapiens
  misc_feature 1..25: note = GNA11HSS104213
  source 1..25: mol_type = mRNA
gcatcagtacgtcagtgccatcaag

SEQ ID NO: 101 - RNA, 25 nt, Homo sapiens
  misc_feature 1..25: note = GNA12MSS204749
  source 1..25: mol_type = mRNA
ccatcgtcaacaacaagctcttctt

SEQ ID NO: 102 - RNA, 25 nt, Homo sapiens
  misc_feature 1..25: note = GNA13-HSS173827
  source 1..25: mol_type = mRNA
cagaagcccttataccaccacttca

SEQ ID NO: 103 - RNA, 25 nt, Homo sapiens
  misc_feature 1..25: note = GNA13-HSS116479
  source 1..25: mol_type = mRNA
gcagcccaaggaatggtggaaacaa

SEQ ID NO: 104 - RNA, 25 nt, Homo sapiens
  misc_feature 1..25: note = ADAM17-HSS186181
  source 1..25: mol_type = mRNA
cagaatcgtgttgacagcaaagaaa

SEQ ID NO: 105 - DNA, 21 bp, Homo sapiens
  misc_feature 1..21: note = GNAQ_1
  source 1..21: mol_type = genomic DNA
accgaatggaggaaagcaagg

SEQ ID NO: 106 - DNA, 25 bp, Homo sapiens
  misc_feature 1..25: note = GNAQ2
  source 1..25: mol_type = genomic DNA
catctctctggggtccatcatattc

SEQ ID NO: 107 - DNA, 20 bp, Homo sapiens
  misc_feature 1..20: note = GNA11_1
  source 1..20: mol_type = genomic DNA
cagcgaatacgaccaagtcc

SEQ ID NO: 108 - DNA, 20 bp, Homo sapiens
  misc_feature 1..20: note = GNA11_2
  source 1..20: mol_type = genomic DNA
accaggggtaggtgatgatg

SEQ ID NO: 109 - DNA, 19 bp, Homo sapiens
  misc_feature 1..19: note = GNA12_1
  source 1..19: mol_type = genomic DNA
gagggattctggcatcagg

SEQ ID NO: 110 - DNA, 19 bp, Homo sapiens
  misc_feature 1..19: note = GNA12_2
  source 1..19: mol_type = genomic DNA
cgatccggtccaagttgtc

SEQ ID NO: 111 - DNA, 24 bp, Homo sapiens
  misc_feature 1..24: note = GNA13_1
  source 1..24: mol_type = genomic DNA
cctggataacttggataaacttgg

SEQ ID NO: 112 - DNA, 19 bp, Homo sapiens
  misc_feature 1..19: note = GNA13_2
  source 1..19: mol_type = genomic DNA
ttcatggatgcctttggtg

SEQ ID NO: 113 - DNA, 22 bp, Homo sapiens
  misc_feature 1..22: note = GAPDH_1
  source 1..22: mol_type = genomic DNA
gccaaggtcatccatgacaact

SEQ ID NO: 114 - DNA, 20 bp, Homo sapiens
  misc_feature 1..20: note = GADPH_2
  source 1..20: mol_type = genomic DNA
gaggggccatccacagtctt